(19) United States
(12) Patent Application Publication
(10) Pub. No.: US 2025/0170373 A1
(43) Pub. Date: May 29, 2025

(54) GALLBLADDER DEFUNCTIONALIZATION DEVICES AND METHODS

(71) Applicant: Texas Medical Center, Houston, TX (US)

(72) Inventors: Benjamin Morgan LEGUM, Houston, TX (US); Shadi SHARIATNIA, Houston, TX (US); Matthew NOJOOMI, Houston, TX (US); Kovi BESSOFF, Palo Alto, CA (US)

(21) Appl. No.: 18/757,398

(22) Filed: Jun. 27, 2024

Related U.S. Application Data
(63) Continuation of application No. 17/865,469, filed on Jul. 15, 2022, now abandoned.
Application No. 17/865,469 is a continuation of application No. 16/988,028, filed on Aug. 7, 2020, now abandoned.
Application No. 16/988,028 is a continuation of application No. PCT/US2019/017112, filed on Feb. 7, 2019.
(60) Provisional application No. 62/667,244, filed on May 4, 2018.
(60) Provisional application No. 62/628,217, filed on Feb. 8, 2018.

(51) Int. Cl. A61M 25/10; A61B 18/00; A61B 18/02; A61B 18/04; A61B 18/12; A61B 18/14; A61B 90/00; A61M 25/00; A61M 25/06

(52) U.S. Cl. CPC A61M 25/10; A61B 18/02; A61B 18/04; A61B 18/1492; A61B 2018/0022; A61B 2018/00535; A61B 2018/00577; A61B 2018/00589; A61B 2018/00666; A61B 2018/00744; A61B 2018/00791; A61B 2018/0212; A61B 18/0218; A61B 2018/0262; A61B 2018/044; A61B 2018/126; A61B 2090/064; A61B 2218/007; A61M 2025/0002; A61M 2025/0681

(57) ABSTRACT

Provided herein is an apparatus having a first tubular body, a second tubular body disposable within the first tubular body, a first plurality of fenestrations in fluid communication with a gallbladder lumen, and an expandable body disposed around the first plurality of fenestrations. The first plurality of fenestrations is configured to deliver a phase changing ablation medium by spraying the phase changing ablation medium in a spatially diffuse pattern into the space defined by the expandable body between the first plurality of fenestrations and the wall of the gallbladder. The first tubular body and the second tubular body define an annular flow path. A pressure sensor measures intraluminal pressure of the gallbladder. A control unit is coupled to the pressure sensor.

### CROSS-REFERENCE TO RELATED APPLICATIONS

This application is a continuation of U.S. patent application Ser. No. 17/865,469, filed Jul. 15, 2022, titled “Catheter Devices for Defunctionalization of a Gallbladder, and Systems and Methods Thereof,” which is a continuation of U.S. patent application Ser. No. 16/988,028, filed Aug. 7, 2020, titled “Gallbladder Defunctionalization Devices and Methods,” now abandoned, which is a continuation of PCT/US2019/017112, filed Feb. 7, 2019, titled “Gallbladder Defunctionalization Devices and Methods,” which claims the benefit of U.S. Provisional Application No. 62/628,217, filed Feb. 8, 2018, titled “Gallbladder Defunctionalization Devices and Methods,” and U.S. Provisional Application No. 62/667,244, filed May 4, 2018, titled “Gallbladder Defunctionalization Devices and Methods.” The disclosures of U.S. patent application Ser. No. 17/865,469, U.S. patent application Ser. No. 16/988,028, International Patent Application No. PCT/US2019/017112, U.S. Provisional Application No. 62/628,217, and U.S. Provisional Application No. 62/667,244 are incorporated herein by reference.

### SUMMARY OF THE DISCLOSURE

The present disclosure relates to devices and methods for defunctionalization of a gallbladder.

Disclosed herein, in certain embodiments, are systems for defunctionalization of a gallbladder in a subject in need thereof, comprising: an access sheath having a first proximal end, a first distal end, a first tubular body therebetween, and a first lumen therein, the first lumen of the access sheath in fluid communication with an evacuator; the access sheath comprising: a seal extending along the circumference of the access sheath at the first distal end of the access sheath; a catheter having a second proximal end, a second distal end, a second tubular body therebetween, and a second lumen therein, the catheter located within the first lumen of the access sheath, and being extendable beyond the first distal end of the access sheath; the catheter comprising: a plurality of fenestrations located at the second distal end of the catheter, the plurality of fenestrations defining a plurality of ablation medium flow paths out of the second tubular body of the catheter and extending along a surface of the catheter in a circumferential pattern; and a connection to an ablation medium supply, the connection providing a fluid communication of an ablation medium with the plurality of fenestrations; a pressure sensor configured to detect an intraluminal pressure in the gallbladder; an extracorporeal control unit operatively connected to the pressure sensor and to the evacuator, the extracorporeal control unit configured to selectively direct an evacuation of the ablation medium through the first lumen of the access sheath upon reaching a pressure threshold.

In some embodiments, the access sheath further comprises a balloon tamponade configured to minimize bleeding in a tissue surrounding the access sheath. In some embodiments, the balloon tamponade is coated with a procoagulant material. In some embodiments, the access sheath further comprises a radiofrequency ablater configured to minimize bleeding and induce scarring in a tissue surrounding the access sheath. In some embodiments, the ablation medium is a thermal ablation medium. In some embodiments, the ablation medium is a cryogenic ablation medium. In some embodiments, the cryogenic ablation medium is nitrous oxide. In some embodiments, the cryogenic ablation medium undergoes a liquid-to-gas phase transition at a phase change interface of the catheter. In some embodiments, the phase change interface of the catheter is an area of the catheter where the second lumen of the catheter decreases in diameter size. In some embodiments, the extracorporeal control unit comprises a connection for a visual output for a user. In some embodiments, the visual output is a digital output or an analog output. In some embodiments, the visual output comprises a temperature measurement, a pressure measurement, or a combination thereof. In some embodiments, the extracorporeal control unit further comprises a fluid collection system configured to collect the ablation medium, a body fluid, a gallstone, a gallstone fragment, or any combination thereof. In some embodiments, the extracorporeal control unit is operatively connected to the ablation medium supply. In some embodiments, the extracorporeal control unit is configured to selectively direct delivery of the ablation medium through the plurality of fenestrations upon reaching a temperature threshold or a pressure threshold. In some embodiments, the evacuator is a vacuum pump that generates a suction force. In some embodiments, the evacuation of the ablation medium is an active evacuation pulling negative pressure through the first lumen of the access sheath. In some embodiments, the plurality of fenestrations extends along the surface of the catheter in a longitudinally directed pattern. In some embodiments, the pattern is pattern is a linear pattern, a hexagonal pattern, a rectangular pattern, a triangular pattern, a square pattern, a circular pattern, a spiral pattern, or any combination thereof. In some embodiments, the plurality of fenestrations extends the surface of the catheter for a length ranging from about 1 centimeter to about 10 centimeters. In some embodiments, the diameter of each of the fenestrations ranges from about 0.001 centimeters to about 0.5 centimeters.

In some embodiments, the system further comprises a cystic duct occluder that occludes a cystic duct, blocks a flow of bile through the cystic duct, or any combination thereof. In some embodiments, the cystic duct occluder is a temporary cystic duct occluder. In some embodiments, the temporary cystic duct occluder is a plug. In some embodiments, the plug is a bioresorbable plug, a degradable plug, a tapered plug, an inflatable plug, a threaded plug, a tissue ingrowth plug, a coil plug, an adhesive plug, a one-way valve plug, or any combination thereof. In some embodiments, the cystic duct occluder is a permanent cystic duct occluder. In some embodiments, the permanent cystic duct occluder is an ablation medium. In some embodiments, the permanent cystic duct occluder is an ablation balloon. In some embodiments, the permanent cystic duct occluder is a radiofrequency ablater. In some embodiments, the system further comprises an ablation balloon. In some embodiments, the ablation balloon comprises an ablation medium. In some embodiments, the ablation medium is a thermal conductive ablation medium or a cryogenic conductive ablation medium. In some embodiments, the ablation balloon is configured to conductively ablate a surrounding tissue. In some embodiments, the ablation balloon is a fenestrated ablation balloon. In some embodiments, the fenestrated ablation balloon comprises an ablation medium. In some embodiments, the ablation medium is a thermal conductive ablation medium or a cryogenic conductive ablation medium. In some embodiments, the fenestrated ablation balloon is configured to convectively ablate a surrounding tissue.

In some embodiments, the system further comprises a radiofrequency ablater located at the second distal end of the catheter, the radiofrequency ablater configured to ablate a tissue via heat transfer. In some embodiments, the radiofrequency ablater comprises at least one electrode that generates heat when energized. In some embodiments, the system further comprises a temperature sensor is located at the first distal end of the system, in fluid connection with a lumen of the gallbladder, when in use. In some embodiments, the temperature sensor is configured to detect a temperature of the ablation medium in the gallbladder, of a fluid in the gallbladder, or a combination thereof. In some embodiments, the pressure threshold ranges from about 30 mmHg to about 40 mmHg.

Disclosed herein, in certain embodiments, are systems for defunctionalization of a gallbladder in a subject in need thereof, comprising: an access sheath having a first proximal end, a first distal end, a first tubular body therebetween, and a first lumen therein, the first lumen of the access sheath in fluid communication with an evacuator; the access sheath comprising: a seal extending along the circumference of the access sheath at the first distal end of the access sheath; and a catheter having a second proximal end, a second distal end, a second tubular body therebetween, and a second lumen therein, the catheter located within the first lumen of the access sheath, and being extendable beyond the first distal end of the access sheath; the catheter comprising: a plurality of fenestrations located at the second distal end of the catheter, the plurality of fenestrations defining a plurality of ablation medium flow paths out of the second tubular body of the catheter and extending along a surface of the catheter in a circumferential pattern; and a connection to an ablation medium supply, the connection providing a fluid communication of an ablation medium with the plurality of fenestrations.

In some embodiments, the access sheath further comprises a balloon tamponade configured to minimize bleeding in a tissue surrounding the access sheath. In some embodiments, the balloon tamponade is coated with a procoagulant material. In some embodiments, the access sheath further comprises a radiofrequency ablater configured to minimize bleeding and induce scarring in a tissue surrounding the access sheath. In some embodiments, the ablation medium is a thermal ablation medium. In some embodiments, the ablation medium is a cryogenic ablation medium. In some embodiments, the cryogenic ablation medium is nitrous oxide. In some embodiments, the cryogenic ablation medium undergoes a liquid-to-gas phase transition at a phase change interface of the catheter. In some embodiments, the phase change interface of the catheter is an area of the catheter where the second lumen of the catheter decreases in diameter size. In some embodiments, the system further comprises a pressure sensor configured to detect an intraluminal pressure in the gallbladder. In some embodiments, the system further comprises an extracorporeal control unit that is operatively connected to the pressure sensor. In some embodiments, the extracorporeal control unit is configured to display the intraluminal pressure. In some embodiments, the extracorporeal control unit comprises a connection for a visual output for a user. In some embodiments, the visual output is a digital output or an analog output. In some embodiments, the visual output comprises a temperature measurement, a pressure measurement, or a combination thereof. In some embodiments, the extracorporeal control unit further comprises a fluid collection system configured to collect the ablation medium, a body fluid, a gallstone, a gallstone fragment, or any combination thereof. In some embodiments, the extracorporeal control unit is operatively connected to the ablation medium supply. In some embodiments, an evacuation of the ablation medium is a passive evacuation that is not selectively directed by the extracorporeal control unit. In some embodiments, the passive evacuation of the ablation medium comprises draining of the ablation medium caused by a pressure gradient, wherein the ablation medium in gallbladder is at a higher pressure than atmospheric pressure, thereby generating the pressure gradient. In some embodiments, the plurality of fenestrations extends along the surface of the catheter in a longitudinally directed pattern.

In some embodiments, the pattern is pattern is a linear pattern, a hexagonal pattern, a rectangular pattern, a triangular pattern, a square pattern, a circular pattern, a spiral pattern, or any combination thereof. In some embodiments, the plurality of fenestrations extends the surface of the catheter for a length ranging from about 1 centimeter to about 10 centimeters. In some embodiments, the diameter of each of the fenestrations ranges from about 0.001 centimeters to about 0.5 centimeters. In some embodiments, the system further comprises a cystic duct occluder that occludes a cystic duct, blocks a flow of bile through the cystic duct, or any combination thereof. In some embodiments, the cystic duct occluder is a temporary cystic duct occluder. In some embodiments, the temporary cystic duct occluder is a plug. In some embodiments, the plug is a bioresorbable plug, a degradable plug, a tapered plug, an inflatable plug, a threaded plug, a tissue ingrowth plug, a coil plug, an adhesive plug, a one-way valve plug, or any combination thereof. In some embodiments, the cystic duct occluder is a permanent cystic duct occluder. In some embodiments, the permanent cystic duct occluder is an ablation medium. In some embodiments, the permanent cystic duct occluder is an ablation balloon. In some embodiments, the permanent cystic duct occluder is a radiofrequency ablater.

In some embodiments, the system further comprises an ablation balloon. In some embodiments, the ablation balloon comprises an ablation medium. In some embodiments, the ablation medium is a thermal conductive ablation medium or a cryogenic conductive ablation medium. In some embodiments, the ablation balloon is configured to conductively ablate a surrounding tissue. In some embodiments, the ablation balloon is a fenestrated ablation balloon. In some embodiments, the fenestrated ablation balloon comprises an ablation medium. In some embodiments, the ablation medium is a thermal conductive ablation medium or a cryogenic conductive ablation medium. In some embodiments, the fenestrated ablation balloon is configured to convectively ablate a surrounding tissue. In some embodiments, the system further comprises a radiofrequency ablater located at the second distal end of the catheter, the radiofrequency ablater configured to ablate a tissue via heat transfer. In some embodiments, the radiofrequency ablater comprises at least one electrode that generates heat when energized. In some embodiments, the system further comprises a temperature sensor is located at the first distal end of the system, in fluid connection with a lumen of the gallbladder, when in use. In some embodiments, the temperature sensor is configured to detect a temperature of the ablation medium in the gallbladder, of a fluid in the gallbladder, or a combination thereof.

Disclosed herein, in certain embodiments, are systems for defunctionalization of a gallbladder in a subject in need thereof, comprising: an access sheath having a first proximal end, a first distal end, a first tubular body therebetween, and a first lumen therein, the first lumen of the access sheath in fluid communication with an evacuator; the access sheath comprising: a seal extending along the circumference of the access sheath at the first distal end of the access sheath; and a an ablation balloon having a surface, a second expandable body, and a second lumen; the ablation balloon comprising: a first plurality of fenestrations located at the surface of the ablation balloon, the first plurality of fenestrations defining a plurality of ablation medium flow paths out of second lumen of the ablation balloon and extending along the surface of the ablation balloon in a circumferential pattern; and a connection to an ablation medium supply, the connection providing a fluid communication of an ablation medium with the first plurality of fenestrations; a pressure sensor configured to detect an intraluminal pressure in the gallbladder; an extracorporeal control unit operatively connected to the pressure sensor and to the evacuator, the extracorporeal control unit configured to selectively direct an evacuation of the ablation medium through the first lumen of the access sheath upon reaching a pressure threshold.

In some embodiments, the access sheath further comprises a balloon tamponade configured to minimize bleeding in a tissue surrounding the access sheath. In some embodiments, the balloon tamponade is coated with a procoagulant material. In some embodiments, the access sheath further comprises a radiofrequency ablater configured to minimize bleeding and induce scarring in a tissue surrounding the access sheath. In some embodiments, the ablation medium is a thermal ablation medium. In some embodiments, the ablation medium is a cryogenic ablation medium. In some embodiments, the cryogenic ablation medium is nitrous oxide. In some embodiments, the cryogenic ablation medium undergoes a liquid-to-gas phase transition at a phase change interface of the catheter. In some embodiments, the phase change interface of the catheter is an area of the catheter where the second lumen of the catheter decreases in diameter size. In some embodiments, the extracorporeal control unit comprises a connection for a visual output for a user. In some embodiments, the visual output is a digital output or an analog output. In some embodiments, the visual output comprises a temperature measurement, a pressure measurement, or a combination thereof. In some embodiments, the extracorporeal control unit further comprises a fluid collection system configured to collect the ablation medium, a body fluid, a gallstone, a gallstone fragment, or any combination thereof.

In some embodiments, the extracorporeal control unit is operatively connected to the ablation medium supply. In some embodiments, the extracorporeal control unit is configured to selectively direct delivery of the ablation medium through the plurality of fenestrations upon reaching a temperature threshold or a pressure threshold. In some embodiments, the evacuator is a vacuum pump that generates a suction force. In some embodiments, the evacuation of the ablation medium is an active evacuation pulling negative pressure through the first lumen of the access sheath. In some embodiments, the first plurality of fenestrations extends along the surface of the ablation balloon in a longitudinally directed pattern. In some embodiments, the pattern is pattern is a linear pattern, a hexagonal pattern, a rectangular pattern, a triangular pattern, a square pattern, a circular pattern, a spiral pattern, or any combination thereof. In some embodiments, the first plurality of fenestrations extends the surface of the ablation balloon for a length ranging from about 1 centimeter to about 10 centimeters. In some embodiments, the diameter of each of the fenestrations in the first plurality of fenestrations ranges from about 0.001 centimeters to about 0.5 centimeters.

In some embodiments, the system further comprises a catheter having a second proximal end, a second distal end, a third tubular body therebetween, and a third lumen therein. In some embodiments, the catheter comprises an opening. In some embodiments, the second lumen of the ablation balloon is in fluid communication with the opening. In some embodiments, the catheter is located within the first lumen of the access sheath. In some embodiments, the catheter is extendable beyond the first distal end of the access sheath. In some embodiments, the catheter comprises a second plurality of fenestrations located at the second distal end of the catheter. In some embodiments, the second plurality of fenestrations defines a plurality of ablation medium flow paths out of the third tubular body of the catheter and extending along a surface of the catheter in a circumferential pattern. In some embodiments, the catheter comprises a connection to the ablation medium supply, the connection providing a fluid communication of the ablation medium with the second plurality of fenestrations. In some embodiments, the second lumen of the ablation balloon is in fluid communication with the second plurality of fenestrations of the catheter. In some embodiments, the second plurality of fenestrations extends along the surface of the catheter in a longitudinally directed pattern.

In some embodiments, the pattern is pattern is a linear pattern, a hexagonal pattern, a rectangular pattern, a triangular pattern, a square pattern, a circular pattern, a spiral pattern, or any combination thereof. In some embodiments, the second plurality of fenestrations extends the surface of the catheter for a length ranging from about 1 centimeter to about 10 centimeters. In some embodiments, the diameter of each of the fenestrations in the second plurality of fenestrations ranges from about 0.001 centimeters to about 0.5 centimeters. In some embodiments, the system further comprises a cystic duct occluder that occludes a cystic duct, blocks a flow of bile through the cystic duct, or any combination thereof. In some embodiments, the cystic duct occluder is a temporary cystic duct occluder. In some embodiments, the temporary cystic duct occluder is a plug. In some embodiments, the plug is a bioresorbable plug, a degradable plug, a tapered plug, an inflatable plug, a threaded plug, a tissue ingrowth plug, a coil plug, an adhesive plug, a one-way valve plug, or any combination thereof. In some embodiments, the cystic duct occluder is a permanent cystic duct occluder. In some embodiments, the permanent cystic duct occluder is an ablation medium. In some embodiments, the permanent cystic duct occluder is an ablation balloon. In some embodiments, the permanent cystic duct occluder is a radiofrequency ablater. In some embodiments, the ablation balloon comprises the ablation medium. In some embodiments, the ablation medium is a thermal conductive ablation medium or a cryogenic conductive ablation medium. In some embodiments, the ablation balloon is configured to convectively ablate a surrounding tissue.

In some embodiments, the system further comprises a radiofrequency ablater located at the second distal end of the catheter, the radiofrequency ablater configured to ablate a tissue via heat transfer. In some embodiments, the radiofrequency ablater comprises at least one electrode that generates heat when energized. In some embodiments, the system further comprises a temperature sensor is located at the first distal end of the system, in fluid connection with a lumen of the gallbladder, when in use. In some embodiments, the temperature sensor is configured to detect a temperature of the ablation medium in the gallbladder, of a fluid in the gallbladder, or a combination thereof. In some embodiments, the pressure threshold ranges from about 30 mmHg to about 40 mmHg.

Disclosed herein, in certain embodiments, are systems for defunctionalization of a gallbladder in a subject in need thereof, comprising: an access sheath having a first proximal end, a first distal end, a first tubular body therebetween, and a first lumen therein, the first lumen of the access sheath in fluid communication with an evacuator; the access sheath comprising: a seal extending along the circumference of the access sheath at the first distal end of the access sheath; and a an ablation balloon having a surface, a second expandable body, and a second lumen; the ablation balloon comprising: a first plurality of fenestrations located at the surface of the ablation balloon, the first plurality of fenestrations defining a plurality of ablation medium flow paths out of second lumen of the ablation balloon and extending along the surface of the ablation balloon in a circumferential pattern; and a connection to an ablation medium supply, the connection providing a fluid communication of an ablation medium with the first plurality of fenestrations.

In some embodiments, the access sheath further comprises a balloon tamponade configured to minimize bleeding in a tissue surrounding the access sheath. In some embodiments, the balloon tamponade is coated with a procoagulant material. In some embodiments, the access sheath further comprises a radiofrequency ablater configured to minimize bleeding and induce scarring in a tissue surrounding the access sheath. In some embodiments, the ablation medium is a thermal ablation medium. In some embodiments, the ablation medium is a cryogenic ablation medium. In some embodiments, the cryogenic ablation medium is nitrous oxide. In some embodiments, the cryogenic ablation medium undergoes a liquid-to-gas phase transition at a phase change interface of the catheter. In some embodiments, the phase change interface of the catheter is an area of the catheter where the second lumen of the catheter decreases in diameter size. In some embodiments, the extracorporeal control unit comprises a connection for a visual output for a user. In some embodiments, the visual output is a digital output or an analog output. In some embodiments, the visual output comprises a temperature measurement, a pressure measurement, or a combination thereof.

In some embodiments, the system further comprises a pressure sensor configured to detect an intraluminal pressure in the gallbladder. In some embodiments, the system further comprises an extracorporeal control unit that is operatively connected to the pressure sensor. In some embodiments, the extracorporeal control unit is configured to display the intraluminal pressure. In some embodiments, the extracorporeal control unit comprises a connection for a visual output for a user. In some embodiments, the visual output is a digital output or an analog output. In some embodiments, the visual output comprises a temperature measurement, a pressure measurement, or a combination thereof. In some embodiments, the extracorporeal control unit further comprises a fluid collection system configured to collect the ablation medium, a body fluid, a gallstone, a gallstone fragment, or any combination thereof. In some embodiments, the extracorporeal control unit is operatively connected to the ablation medium supply. In some embodiments, an evacuation of the ablation medium is a passive evacuation that is not selectively directed by the extracorporeal control unit. In some embodiments, the passive evacuation of the ablation medium comprises draining of the ablation medium caused by a pressure gradient, wherein the ablation medium in gallbladder is at a higher pressure than atmospheric pressure, thereby generating the pressure gradient. In some embodiments, the first plurality of fenestrations extends along the surface of the ablation balloon in a longitudinally directed pattern. In some embodiments, the pattern is pattern is a linear pattern, a hexagonal pattern, a rectangular pattern, a triangular pattern, a square pattern, a circular pattern, a spiral pattern, or any combination thereof. In some embodiments, the first plurality of fenestrations extends the surface of the ablation balloon for a length ranging from about 1 centimeter to about 10 centimeters. In some embodiments, the diameter of each of the fenestrations in the first plurality of fenestrations ranges from about 0.001 centimeters to about 0.5 centimeters.

In some embodiments, the system further comprises a catheter having a second proximal end, a second distal end, a third tubular body therebetween, and a third lumen therein. In some embodiments, the catheter comprises an opening. In some embodiments, the second lumen of the ablation balloon is in fluid communication with the opening. In some embodiments, the catheter is located within the first lumen of the access sheath. In some embodiments, the catheter is extendable beyond the first distal end of the access sheath. In some embodiments, the catheter comprises a second plurality of fenestrations located at the second distal end of the catheter. In some embodiments, the second plurality of fenestrations defines a plurality of ablation medium flow paths out of the third tubular body of the catheter and extending along a surface of the catheter in a circumferential pattern. In some embodiments, the catheter comprises a connection to the ablation medium supply, the connection providing a fluid communication of the ablation medium with the second plurality of fenestrations. In some embodiments, the second lumen of the ablation balloon is in fluid communication with the second plurality of fenestrations of the catheter.

In some embodiments, the second plurality of fenestrations extends along the surface of the catheter in a longitudinally directed pattern. In some embodiments, the pattern is pattern is a linear pattern, a hexagonal pattern, a rectangular pattern, a triangular pattern, a square pattern, a circular pattern, a spiral pattern, or any combination thereof. In some embodiments, the second plurality of fenestrations extends the surface of the catheter for a length ranging from about 1 centimeter to about 10 centimeters. In some embodiments, the diameter of each of the fenestrations in the second plurality of fenestrations ranges from about 0.001 centimeters to about 0.5 centimeters. In some embodiments, the system further comprises a cystic duct occluder that occludes a cystic duct, blocks a flow of bile through the cystic duct, or any combination thereof. In some embodiments, the cystic duct occluder is a temporary cystic duct occluder. In some embodiments, the temporary cystic duct occluder is a plug. In some embodiments, the plug is a bioresorbable plug, a degradable plug, a tapered plug, an inflatable plug, a threaded plug, a tissue ingrowth plug, a coil plug, an adhesive plug, a one-way valve plug, or any combination thereof. In some embodiments, the cystic duct occluder is a permanent cystic duct occluder. In some embodiments, the permanent cystic duct occluder is an ablation medium. In some embodiments, the permanent cystic duct occluder is an ablation balloon. In some embodiments, the permanent cystic duct occluder is a radiofrequency ablater. In some embodiments, the ablation balloon comprises the ablation medium. In some embodiments, the ablation medium is a thermal conductive ablation medium or a cryogenic conductive ablation medium. In some embodiments, the ablation balloon is configured to convectively ablate a surrounding tissue.

In some embodiments, the system further comprises a radiofrequency ablater located at the second distal end of the catheter, the radiofrequency ablater configured to ablate a tissue via heat transfer. In some embodiments, the radiofrequency ablater comprises at least one electrode that generates heat when energized. In some embodiments, the system further comprises a temperature sensor is located at the first distal end of the system, in fluid connection with a lumen of the gallbladder, when in use. In some embodiments, the temperature sensor is configured to detect a temperature of the ablation medium in the gallbladder, of a fluid in the gallbladder, or a combination thereof.

Disclosed herein, in certain embodiments, are systems for defunctionalization of a gallbladder in a subject in need thereof, comprising: an access sheath having a first proximal end, a first distal end, a first tubular body therebetween, and a first lumen therein, the first lumen of the access sheath in fluid communication with an evacuator; the access sheath comprising: a seal extending along the circumference of the access sheath at the first distal end of the access sheath; and an ablation balloon having a surface, a second expandable body, and a second lumen, the second lumen in fluid communication with an ablation medium supply; a pressure sensor configured to detect an intraluminal pressure in the gallbladder; an extracorporeal control unit operatively connected to the pressure sensor and to the evacuator, the extracorporeal control unit configured to selectively direct an evacuation of an ablation medium through the first lumen of the access sheath upon reaching a pressure threshold.

In some embodiments, the access sheath further comprises a balloon tamponade configured to minimize bleeding in a tissue surrounding the access sheath. In some embodiments, the balloon tamponade is coated with a procoagulant material. In some embodiments, the access sheath further comprises a radiofrequency ablater configured to minimize bleeding and induce scarring in a tissue surrounding the access sheath. In some embodiments, the ablation medium is a thermal ablation medium. In some embodiments, the ablation medium is a cryogenic ablation medium. In some embodiments, the cryogenic ablation medium is nitrous oxide. In some embodiments, the cryogenic ablation medium undergoes a liquid-to-gas phase transition at a phase change interface of the catheter. In some embodiments, the phase change interface of the catheter is an area of the catheter where the second lumen of the catheter decreases in diameter size. In some embodiments, the extracorporeal control unit comprises a connection for a visual output for a user. In some embodiments, the visual output is a digital output or an analog output. In some embodiments, the visual output comprises a temperature measurement, a pressure measurement, or a combination thereof. In some embodiments, the extracorporeal control unit further comprises a fluid collection system configured to collect the ablation medium, a body fluid, a gallstone, a gallstone fragment, or any combination thereof. In some embodiments, the extracorporeal control unit is operatively connected to the ablation medium supply. In some embodiments, the extracorporeal control unit is configured to selectively direct delivery of the ablation medium through the plurality of fenestrations upon reaching a temperature threshold or a pressure threshold.

In some embodiments, the evacuator is a vacuum pump that generates a suction force. In some embodiments, the evacuation of the ablation medium is an active evacuation pulling negative pressure through the first lumen of the access sheath. In some embodiments, the system further comprises a catheter having a second proximal end, a second distal end, a third tubular body therebetween, and a third lumen therein. In some embodiments, the catheter comprises an opening. In some embodiments, the second lumen of the ablation balloon is in fluid communication with the opening. In some embodiments, the catheter is located within the first lumen of the access sheath. In some embodiments, the catheter is extendable beyond the first distal end of the access sheath. In some embodiments, the catheter comprises a plurality of fenestrations located at the second distal end of the catheter. In some embodiments, the plurality of fenestrations defines a plurality of ablation medium flow paths out of the third tubular body of the catheter and extending along a surface of the catheter in a circumferential pattern. In some embodiments, the catheter comprises a connection to the ablation medium supply, the connection providing a fluid communication of the ablation medium with the plurality of fenestrations. In some embodiments, the second lumen of the ablation balloon is in fluid communication with the plurality of fenestrations of the catheter. In some embodiments, the plurality of fenestrations extends along the surface of the catheter in a longitudinally directed pattern. In some embodiments, the pattern is pattern is a linear pattern, a hexagonal pattern, a rectangular pattern, a triangular pattern, a square pattern, a circular pattern, a spiral pattern, or any combination thereof. In some embodiments, the plurality of fenestrations extends the surface of the catheter for a length ranging from about 1 centimeter to about 10 centimeters.

In some embodiments, the diameter of each of the fenestrations in the plurality of fenestrations ranges from about 0.001 centimeters to about 0.5 centimeters. In some embodiments, the system further comprises a cystic duct occluder that occludes a cystic duct, blocks a flow of bile through the cystic duct, or any combination thereof. In some embodiments, the cystic duct occluder is a temporary cystic duct occluder. In some embodiments, the temporary cystic duct occluder is a plug. In some embodiments, the plug is a bioresorbable plug, a degradable plug, a tapered plug, an inflatable plug, a threaded plug, a tissue ingrowth plug, a coil plug, an adhesive plug, a one-way valve plug, or any combination thereof. In some embodiments, the cystic duct occluder is a permanent cystic duct occluder. In some embodiments, the permanent cystic duct occluder is an ablation medium. In some embodiments, the permanent cystic duct occluder is an ablation balloon. In some embodiments, the permanent cystic duct occluder is a radiofrequency ablater. In some embodiments, the ablation balloon comprises the ablation medium. In some embodiments, the ablation medium is a thermal conductive ablation medium or a cryogenic conductive ablation medium. In some embodiments, the ablation balloon is configured to conductively ablate a surrounding tissue. In some embodiments, the system further comprises a radiofrequency ablater located at the second distal end of the catheter, the radiofrequency ablater configured to ablate a tissue via heat transfer. In some embodiments, the radiofrequency ablater comprises at least one electrode that generates heat when energized. In some embodiments, the system further comprises a temperature sensor is located at the first distal end of the system, in fluid connection with a lumen of the gallbladder, when in use. In some embodiments, the temperature sensor is configured to detect a temperature of the ablation medium in the gallbladder, of a fluid in the gallbladder, or a combination thereof. In some embodiments, the pressure threshold ranges from about 30 mmHg to about 40 mmHg.

Disclosed herein, in certain embodiments, are systems for defunctionalization of a gallbladder in a subject in need thereof, comprising: an access sheath having a first proximal end, a first distal end, a first tubular body therebetween, and a first lumen therein, the first lumen of the access sheath in fluid communication with an evacuator; the access sheath comprising: a seal extending along the circumference of the access sheath at the first distal end of the access sheath; and an ablation balloon having a surface, a second expandable body, and a second lumen, the second lumen in fluid communication with an ablation medium supply.

In some embodiments, the access sheath further comprises a balloon tamponade configured to minimize bleeding in a tissue surrounding the access sheath. In some embodiments, the balloon tamponade is coated with a procoagulant material. In some embodiments, the access sheath further comprises a radiofrequency ablater configured to minimize bleeding and induce scarring in a tissue surrounding the access sheath. In some embodiments, the ablation medium is a thermal ablation medium. In some embodiments, the ablation medium is a cryogenic ablation medium. In some embodiments, the cryogenic ablation medium is nitrous oxide. In some embodiments, the cryogenic ablation medium undergoes a liquid-to-gas phase transition at a phase change interface of the catheter. In some embodiments, the phase change interface of the catheter is an area of the catheter where the second lumen of the catheter decreases in diameter size. In some embodiments, the extracorporeal control unit comprises a connection for a visual output for a user. In some embodiments, the visual output is a digital output or an analog output. In some embodiments, the visual output comprises a temperature measurement, a pressure measurement, or a combination thereof. In some embodiments, the system further comprises a pressure sensor configured to detect an intraluminal pressure in the gallbladder. In some embodiments, the system further comprises an extracorporeal control unit that is operatively connected to the pressure sensor.

In some embodiments, the extracorporeal control unit is configured to display the intraluminal pressure. In some embodiments, the extracorporeal control unit comprises a connection for a visual output for a user. In some embodiments, the visual output is a digital output or an analog output. In some embodiments, the visual output comprises a temperature measurement, a pressure measurement, or a combination thereof. In some embodiments, the extracorporeal control unit further comprises a fluid collection system configured to collect the ablation medium, a body fluid, a gallstone, a gallstone fragment, or any combination thereof. In some embodiments, the extracorporeal control unit is operatively connected to the ablation medium supply. In some embodiments, an evacuation of the ablation medium is a passive evacuation that is not selectively directed by the extracorporeal control unit. In some embodiments, the passive evacuation of the ablation medium comprises draining of the ablation medium caused by a pressure gradient, wherein the ablation medium in gallbladder is at a higher pressure than atmospheric pressure, thereby generating the pressure gradient. In some embodiments, the system further comprises a catheter having a second proximal end, a second distal end, a third tubular body therebetween, and a third lumen therein. In some embodiments, the catheter comprises an opening. In some embodiments, the second lumen of the ablation balloon is in fluid communication with the opening. In some embodiments, the catheter is located within the first lumen of the access sheath. In some embodiments, the catheter is extendable beyond the first distal end of the access sheath. In some embodiments, the catheter comprises a plurality of fenestrations located at the second distal end of the catheter.

In some embodiments, the second plurality of fenestrations defines a plurality of ablation medium flow paths out of the third tubular body of the catheter and extending along a surface of the catheter in a circumferential pattern. In some embodiments, the catheter comprises a connection to the ablation medium supply, the connection providing a fluid communication of the ablation medium with the plurality of fenestrations. In some embodiments, the second lumen of the ablation balloon is in fluid communication with the plurality of fenestrations of the catheter. In some embodiments, the plurality of fenestrations extends along the surface of the catheter in a longitudinally directed pattern. In some embodiments, the pattern is pattern is a linear pattern, a hexagonal pattern, a rectangular pattern, a triangular pattern, a square pattern, a circular pattern, a spiral pattern, or any combination thereof. In some embodiments, the plurality of fenestrations extends the surface of the catheter for a length ranging from about 1 centimeter to about 10 centimeters. In some embodiments, wherein the diameter of each of the fenestrations in the plurality of fenestrations ranges from about 0.001 centimeters to about 0.5 centimeters.

In some embodiments, further comprises a cystic duct occluder that occludes a cystic duct, blocks a flow of bile through the cystic duct, or any combination thereof. In some embodiments, the cystic duct occluder is a temporary cystic duct occluder. In some embodiments, the temporary cystic duct occluder is a plug. In some embodiments, the plug is a bioresorbable plug, a degradable plug, a tapered plug, an inflatable plug, a threaded plug, a tissue ingrowth plug, a coil plug, an adhesive plug, a one-way valve plug, or any combination thereof. In some embodiments, the cystic duct occluder is a permanent cystic duct occluder. In some embodiments, the permanent cystic duct occluder is an ablation medium. In some embodiments, the permanent cystic duct occluder is an ablation balloon. In some embodiments, the permanent cystic duct occluder is a radiofrequency ablater. In some embodiments, the ablation balloon comprises the ablation medium. In some embodiments, the ablation medium is a thermal conductive ablation medium or a cryogenic conductive ablation medium. In some embodiments, the ablation balloon is configured to convectively ablate a surrounding tissue. In some embodiments, further comprising a radiofrequency ablater located at the second distal end of the catheter, the radiofrequency ablater configured to ablate a tissue via heat transfer. In some embodiments, the radiofrequency ablater comprises at least one electrode that generates heat when energized. In some embodiments, the system further comprises a temperature sensor is located at the first distal end of the system, in fluid connection with a lumen of the gallbladder, when in use. In some embodiments, the temperature sensor is configured to detect a temperature of the ablation medium in the gallbladder, of a fluid in the gallbladder, or a combination thereof.

Disclosed herein, in certain embodiments, are devices for defunctionalization of a gallbladder in a subject in need thereof, comprising: a catheter having a proximal end, a distal end, a tubular body therebetween, and a lumen; the catheter comprising: a plurality of fenestrations located at the second distal end of the catheter, the plurality of fenestrations defining a plurality of ablation medium flow paths out of the second tubular body of the catheter and extending along a surface of the catheter in a circumferential pattern; and a connection to an ablation medium supply, the connection providing a fluid communication of an ablation medium with the plurality of fenestrations.

In some embodiments, the ablation medium is a thermal ablation medium. In some embodiments, the ablation medium is a cryogenic ablation medium. In some embodiments, the cryogenic ablation medium is nitrous oxide. In some embodiments, the cryogenic ablation medium undergoes a liquid-to-gas phase transition upon exiting thorough the plurality of fenestrations. In some embodiments, the ablation medium is passively evacuated from the gallbladder by draining of the ablation medium caused by a pressure gradient, wherein the ablation medium in gallbladder is at a higher pressure than the pressure in the first lumen of the access sheath, thereby generating the pressure gradient. In some embodiments, the plurality of fenestrations extends along the surface of the catheter in a longitudinally directed pattern. In some embodiments, the pattern is pattern is a linear pattern, a hexagonal pattern, a rectangular pattern, a triangular pattern, a square pattern, a circular pattern, a spiral pattern, or any combination thereof. In some embodiments, the plurality of fenestrations extends the surface of the catheter for a length ranging from about 1 centimeter to about 10 centimeters. In some embodiments, the diameter of each of the fenestrations ranges from about 0.001 centimeters to about 0.5 centimeters. In some embodiments, the device further comprises a cystic duct occluder that occludes a cystic duct, blocks a flow of bile through the cystic duct, or any combination thereof. In some embodiments, the cystic duct occluder is a temporary cystic duct occluder. In some embodiments, the temporary cystic duct occluder is a plug.

In some embodiments, the plug is a bioresorbable plug, a degradable plug, a tapered plug, an inflatable plug, a threaded plug, a tissue ingrowth plug, a coil plug, an adhesive plug, a one-way valve plug, or any combination thereof. In some embodiments, the cystic duct occluder is a permanent cystic duct occluder. In some embodiments, the permanent cystic duct occluder is an ablation medium. In some embodiments, the permanent cystic duct occluder is an ablation balloon. In some embodiments, the permanent cystic duct occluder is a radiofrequency ablater. In some embodiments, the device further comprises an ablation balloon. In some embodiments, the ablation balloon comprises an ablation medium. In some embodiments, the ablation medium is configured to ablate a tissue by the application of thermal or cryogenic energy. In some embodiments, the ablation balloon is a fenestrated ablation balloon. In some embodiments, the device further comprises a radiofrequency ablater located at the second distal end of the catheter, the radiofrequency ablater configured to ablate a tissue via heat transfer. In some embodiments, the radiofrequency ablater comprises a first electrode and a second electrode that generate heat when energized.

Disclosed herein, in certain embodiments, are methods for defunctionalizing a gallbladder in a subject in need thereof, comprising: a) extending a catheter beyond a first distal end of an access sheath and into the gallbladder; b) pumping an ablation medium through a lumen of the catheter and through a plurality of fenestrations located at a second distal end of the catheter, wherein the plurality of fenestrations define a plurality of ablation medium flow paths out of a tubular body of the catheter and extend along a surface of the catheter in a circumferential pattern; c) detecting an intraluminal pressure in the gallbladder; and d) selectively directing an evacuation of the ablation medium from the gallbladder upon reaching a pressure threshold.

In some embodiments, the ablation medium is a thermal ablation medium. In some embodiments, the ablation medium is a cryogenic ablation medium. In some embodiments, the cryogenic ablation medium is nitrous oxide. In some embodiments, the cryogenic ablation medium undergoes a liquid-to-gas phase transition upon exiting thorough the plurality of fenestrations. In some embodiments, the plurality of fenestrations extends along the surface of the catheter in a longitudinally directed pattern. In some embodiments, the pattern is pattern is a linear pattern, a hexagonal pattern, a rectangular pattern, a triangular pattern, a square pattern, a circular pattern, a spiral pattern, or any combination thereof. In some embodiments, the plurality of fenestrations extends along the surface of the catheter for a length ranging from about 1 centimeter to about 10 centimeters. In some embodiments, the diameter of each of the fenestrations ranges from about 0.001 centimeters to about 0.5 centimeters. In some embodiments, the temperature of the ablation medium in the gallbladder is detected by a temperature sensor. In some embodiments, the pressure of the ablation medium in the gallbladder is detected by a pressure sensor. In some embodiments, the evacuation of the ablation medium is an active evacuation pulling negative pressure through the first lumen of the access sheath. In some embodiments, the evacuation of the ablation medium is a passive evacuation comprising draining of the ablation medium caused by a pressure gradient, wherein the ablation medium in gallbladder is at a higher pressure than the pressure in the first lumen of the access sheath, thereby generating the pressure gradient. In some embodiments, the ablation medium defunctionalizes the gallbladder by inducing tissue necrosis. In some embodiments, the method further comprises detecting an intraluminal temperature of the gallbladder. In some embodiments, the threshold pressure ranges from about 30 mmHg to about 40 mmHg.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1C illustrate different percutaneous and endoscopic access approaches. FIG. 1A illustrates a percutaneous transhepatic access approach. FIG. 1B illustrates a percutaneous subhepatic access approach. FIG. 1C illustrates an endoscopic transmural access approach.

FIGS. 2A-2B illustrate exemplary embodiments of a catheter device. FIG. 2A illustrates a catheter device in the gallbladder with an ablation delivery system, a device access sheath, an extracorporeal control unit, and a cystic duct occluder. FIG. 2B illustrates a catheter device in the gallbladder with an ablation delivery system, a device access sheath, and an extracorporeal control unit.

FIG. 3 illustrates an exemplary embodiment of the catheter device comprising a device access sheath with an extracorporeal control unit, an access seal, a temperature sensor, and a pressure sensor.

FIGS. 4A-4B illustrate exemplary embodiments of the catheter device. FIG. 4A illustrates an embodiment of the catheter device comprising a device access sheath and a balloon tamponade. FIG. 4B illustrates an embodiment of the catheter device comprising a device access sheath and bipolar coagulating electrodes.

FIG. 5 illustrates an embodiment of the catheter device comprising a compliant ablation balloon.

FIG. 6 illustrates an embodiment of the catheter device comprising a fenestrated ablation balloon.

FIGS. 7A-7B illustrates an embodiment of the catheter device comprising a fenestrated nozzle. FIG. 7A illustrates an embodiment of the catheter device comprising a fenestrated nozzle protruding from the device access sheath. FIG. 7B illustrates an embodiment of the catheter device comprising a fenestrated nozzle comprising an adjustable nozzle exposure sheath.

FIG. 8 illustrates an embodiment of the catheter device comprising a catheter comprising an inner cystic duct occlusion catheter containing a pass-through lumen.

FIG. 9 illustrates an embodiment of the catheter device comprising a temporary cystic duct plug and a pair of bipolar coagulating electrodes.

FIGS. 10A-10G illustrate exemplary embodiments of plugs. FIG. 10A illustrates a tapered plug. FIG. 10B illustrates an inflatable plug. FIG. 10C illustrates a threaded plug. FIG. 10D illustrates a tissue ingrowth plug. FIG. 10E illustrates a coil plug. FIG. 10F illustrates an adhesive plug. FIG. 10G illustrates a one-way valve plug.

FIGS. 11A-11C illustrate exemplary embodiments of occluders of the catheter device. FIG. 11A illustrates an ablation spray as an occluder. FIG. 11B illustrates an ablation balloon as an occluder. FIG. 11C illustrates tapered tip with radiofrequency (RF) electrodes as an occluder.

FIG. 12 illustrates a computer system that is programmed or otherwise configured to implement the methods provided herein.

FIG. 13 illustrates a cross-sectional view of the catheter and fenestrated nozzle.

### DETAILED DESCRIPTION OF THE DISCLOSURE

While preferred embodiments of the subject matter disclosed herein have been shown and described herein, it will be obvious to those skilled in the art that such embodiments are provided by way of example only. Numerous variations, changes, and substitutions will now occur to those skilled in the art without departing from the subject matter disclosed herein. It should be understood that various alternatives to the embodiments of the subject matter disclosed herein may be employed in practicing the subject matter disclosed herein. It is intended that the following claims define the scope of the subject matter disclosed herein and that methods and structures within the scope of these claims and their equivalents be covered thereby.

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description or the claims or in both the detailed description and the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, e.g., the limitations of the measurement system. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about” or “approximately” means within 20.0 degrees, 15.0 degrees, 10.0 degrees, 9.0 degrees, 8.0 degrees, 7.0 degrees, 6.0 degrees, 5.0 degrees, 4.0 degrees, 3.0 degrees, 2.0 degrees, 1.0 degrees, 0.9 degrees, 0.8 degrees, 0.7 degrees, 0.6 degrees, 0.5 degrees, 0.4 degrees, 0.3 degrees, 0.2 degrees, 0.1 degrees, 0.09 degrees. 0.08 degrees, 0.07 degrees, 0.06 degrees, 0.05 degrees, 0.04 degrees, 0.03 degrees, 0.02 degrees or 0.01 degrees of a given value or range.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker).

The terms “user,” “health care worker,” “doctor,” “physician,” “provider,” and “health care provider,” are used interchangeably. These terms refer to any person that operates the devices described herein. Additional non-liming examples of a user include “registered nurse,” “nurse practitioner,” and “physician's assistant.”

The term “proximal,” as used herein, is defined as being closest or nearer to the user holding or operating the catheter device, unless otherwise indicated.

The term “distal,” as used herein, is defined as being farthest to or away from the user holding or operating the catheter device, unless otherwise indicated.

The term “occluder,” as used herein is defined as an object, a system, a device, an agent, an ablation medium, or any combination thereof that: 1) partially or completely blocks a duct, a tube, or a passageway of a body; and 2) partially or completely impedes the flow of a fluid, a gas, or any combination thereof between a first organ and a second organ, between a duct, a tube, or a passageway of a first organ and a second organ, or between a proximal end and a distal end of a duct, tube, or a passageway of a body.

The term “ablater,” as used herein is defined as system, a device, an agent, an ablation medium, or any combination thereof that uses an energy source to induce or generate necrosis in a tissue via melting of the tissue, freezing of the tissue, or any combination thereof.

### Cholelithiasis

Gallstones are one of the most common gastrointestinal disorders amongst Americans. Gallstones form when bile, a fluid secreted by the liver and stored in the gallbladder, becomes supersaturated. While they do not cause a problem for many people, gallstones occasionally block the cystic duct, an outlet of the gallbladder, preventing the gallbladder from emptying. In some instances, the obstruction results in pain, inflammation, and infection. In otherwise healthy patients, the gallstone disease is treated by surgical removal of the gallbladder. However, the risks associated with surgical treatment are considerably higher in certain patient populations. For example, 1 in 5 Medicare patients have been shown to suffer an adverse outcome. Non-surgical treatment options for these patients are limited and focus on relieving acute symptoms, without addressing the underlying cause of the disease. In some instances, the disease is likely to recur, resulting in additional clinical risk and significant cost. There currently is no long-term solution for gallbladder disease in high-risk patients.

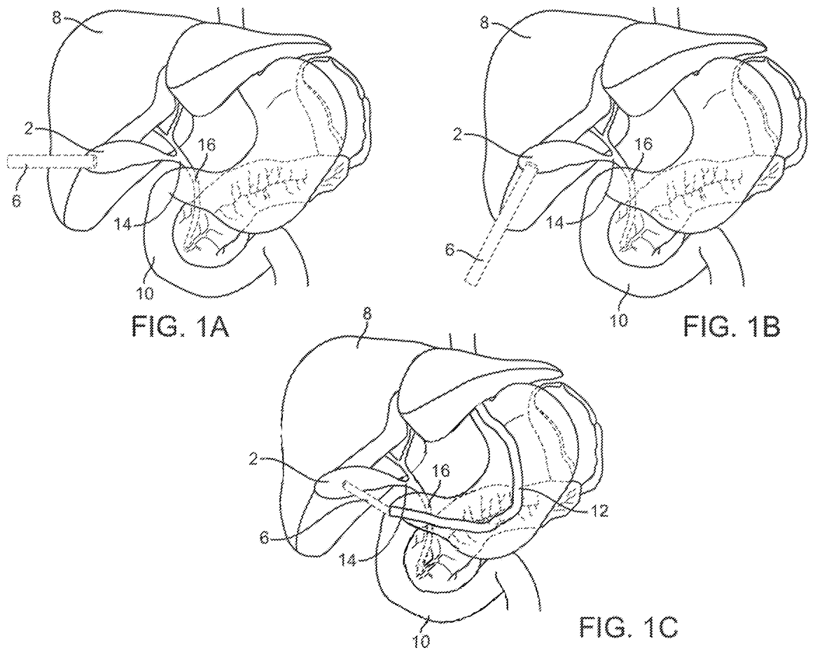

Gallbladder is a small hollow organ in the gastrointestinal system. A blind-ended tubular outpouching of the biliary tree, the gallbladder is a pear-shaped organ with a storage capacity of 30 milliliters (ml)-50 ml. The gallbladder is typically 2-3 centimeters (cm) in breadth and 7-10 cm in axial length. It is typically divided into three parts; the fundus, body, and neck. The neck contains a mucosal fold, known as Hartmann's Pouch, which is a common location for gallstones to become lodged, resulting in cholecystitis. As shown in FIGS. 1A-1C, the gallbladder 2 opens into the cystic duct 14 and connects to the liver 8 by the common hepatic duct 18 which bifurcates into the right hepatic duct and the left hepatic duct. The gallbladder 2 is connected to the small intestine 10 by the common bile duct 16.

The gallbladder stores and concentrates the bile produced by the liver and releases the stored bile into the small intestine, where the bile helps in the digestion of fats in food. Histologically, the gallbladder has 4 layers, including the serosa (the outermost layer), a muscular layer, lamina propria, and the innermost mucosa layer. The mucosal layer of the gallbladder is the innermost layer of the gallbladder wall and concentrates the bile. The serosa is derived from the visceral peritoneum and covers the anterior fundus, body, and neck of the gallbladder. Inside the serosa, a single muscular layer envelopes the lamina propria. The mucosa that lines the inner lumen of the gallbladder is composed of columnar epithelial cells which secrete mucin and dehydrate bile via the action of multiple ion channels. Occasionally, outpouchings (known as Rokitansky-Aschoff nodules) of the mucosa extend into deeper layers of the gallbladder wall.

Bile is made by hepatocytes in the liver and subsequently secreted into hepatic ductules which coalesce into intrahepatic ducts. These ducts converge to form the right and left hepatic ducts which then combine into the common bile duct. The common bile duct joins with the pancreatic duct just proximal to the Ampulla of Vater in the duodenal wall. Bile produced by hepatocytes flows through the biliary system and into the duodenal lumen to aid in digestion. Flow into the duodenal lumen is regulated at the level of the Ampulla of Vater by the Sphincter of Oddi. During an unfed state, when bile is not needed for digestion, the Sphincter is closed, resulting in routing of bile to the gallbladder for storage.

During storage, bile becomes supersaturated, providing a nidus for the formation of gallstones and sludge (very small gallstones). The majority of gallstones are “brown stones”, that are mainly comprised of cholesterol (typically >80%). These stones tend to be brittle and are readily crushed. A minority of stones are predominantly bilirubin (“black stones”; <20% cholesterol) and are often much harder. Mixed stones contain a variable amount of bilirubin and cholesterol.

Mobile gallstones that remain in the lumen of the gallbladder have the potential to cause various pathologies. In some instances, the gallstones become lodged at the neck of the gallbladder, occluding the cystic duct. The lodged gallstones cause gallbladder distension and intermittent right upper quadrant discomfort (likely from intramural muscle spasm at the organ attempts to empty against an increased pressure gradient), a condition known as symptomatic cholelithiasis. In some instances, the gallstones become lodged more permanently at the gallbladder outlet, resulting in inflammation and infection. This is a condition known as cholecystitis, which requires urgent intervention as it can progress to systemic infection. Alternatively or in combination, gallstones or sludge passes through the cystic duct, becoming lodged in the common bile duct, blocking the flow of bile, resulting in a potentially life threatening condition known as ascending cholangitis. In some embodiments, the debris becomes lodged at the confluence of the pancreatic and common bile ducts, causing stagnation of pancreatic secretions, resulting in pancreatitis (inflammation of the pancreas).

In cholelithiasis, supersaturation of bile in gallbladder leads to the formation of gallstones. In some embodiments, impacted gallstones leads to inflammation, pain and infection of the gallbladder. When the gallbladder is inflamed, the mucosal layer of the gallbladder becomes more prominent. In some embodiments, the gallstone disease is diagnosed by ultrasounds or other imaging methods. Provided herein are methods and devices configured to definitively treat benign gallbladder disease in a minimally invasive manner in patients with symptomatic gallstones in order to reduce health care costs and patient morbidity.

Laparoscopic cholecystectomy is a treatment for gallstone disease and is a commonly performed general surgery procedure. During laparoscopic cholecystectomy, small incisions are made in the abdomen, facilitating the removal of the gallbladder with a camera and small instruments. The procedure is safe in otherwise healthy patients, and often does not require hospital admission. In uncomplicated cases, patients are often back to work within two weeks.

In a number of patient populations, the surgical risk associated with laparoscopic cholecystectomy is considerably higher. In some embodiments, these populations include critically ill patients, patients with intra-abdominal scarring from chronic disease and previous surgery, and elderly patients who tend to have a higher incidence of medical comorbidities. One such population is the Medicare population, which comprises approximately 200,000 laparoscopic cholecystectomies per year in the US. Twenty one percent of these surgeries result in an adverse outcome, including prolonged length of stay and readmission and other perioperative complications. In addition to the direct costs associated with these complications, many elderly patients are at risk of not returning to their baseline level of health, resulting in additional healthcare costs.

There are non-surgical options to treat gallstone disease. These include the administration of antibiotics, or placement of a cholecystostomy tube to drain the gallbladder contents, or a combination of the two. However, the non-surgical options do not provide a long-term solution. These options are effective temporizing measures, and they do not treat the cause of the disease. During a percutaneous cholecystostomy, a cholecystostomy tube is placed through the rib cage into the gallbladder. The percutaneous cholecystostomy can take place in an interventional radiology (IR) suite or at the patient's bedside but does not provide a definite treatment of the gallstone disease. Often times, the non-surgical options lead to recurrence and additional hospitalization costs.

For patients with cholecystitis who have a high risk of surgical complications, the treatment is percutaneous decompression of the gallbladder (via a percutaneously inserted cholecystostomy tube) in conjunction with antibiotics. This treatment provides a temporizing measure to allow the patient to recover from the systemic effects of the ongoing infection (sepsis) and return to their baseline state of health (commonly referred to as “cooling off” by healthcare professionals). The cholecystostomy tube remains in place until the patient has recovered. About 6-8 weeks following placement, a cholangiography by injection of radiopaque contrast through the tube under fluoroscopy is performed to determine if the cystic duct is patent (open). The cholecystostomy tube is removed if the cystic duct is patent (open). The treatment is interval cholecystectomy as it reduces the rate of recurrence of the gallstone disease. If there is no communication between the cystic duct and the common bile duct, the tube remains in place until cholecystectomy is performed, or patency is demonstrated on subsequent cholangiography. There is no definitive treatment available for high risk patients, placing them at risk for disease recurrence and exposure to the associated clinical risks and healthcare costs.

Ablation technologies have been used to treat other diseases. For example, ablation has been used in treatment of esophageal metaplasia and endometrial hyperplasia. However, ablation technologies are not readily available for treating gallstone disease. As ablation technologies have not been contemplated for defunctionalization of the gallbladder, they do not include a capability for occlusion of cystic duct. Ablation technologies often are applied to a small targeted area, such as a nerve, and are not typically used for applying to a diffuse area or a tissue or organ.

Provided herein are devices and methods to durably occlude the cystic duct to prevent backflow of bile and re-establishment of functional mucosa and to defunctionalize the gallbladder epithelium to provide definitive treatment for gallstone disease. In some embodiments, the treatment is applicable to patients with gallstone-related disease.

Provided herein are methods and devices for a low-risk treatment for gallstone disease that percutaneously defunctionalizes the gallbladder, instead of surgically removing the gallbladder. This affords patients the benefits of surgical removal of the gallbladder without the risk associated with general anesthesia needed for the surgical removal. The defunctionalization of the gallbladder renders the gallbladder non-functional in storing and releasing bile without removing the gallbladder. In some embodiments, the device for gallbladder defunctionalization comprises an ablation delivery system and a device access sheath. In some embodiments, the ablation delivery system provides energy for ablation, where the energy level, the delivery location, or any combination thereof is controllable and tunable. In some embodiments, the gallbladder defunctionalization device comprises an extracorporeal control unit. In some embodiments, the gallbladder defunctionalization device comprises a cystic duct occluder. In some embodiments, the device access sheath is used to navigate and deliver therapy. In some embodiments, the extracorporeal control unit is used to regulate power requirements. In some embodiments, the extracorporeal control unit is connected to the proximal end of the device access system. In some embodiments, the extracorporeal control unit is connected to the proximal end of the ablation delivery system. In some embodiments, the device access system comprises a catheter configured to percutaneously access the gallbladder. In some embodiments, the device is a handheld device. In some embodiments, the extracorporeal control unit 20 is a handle that interfaces with the device access sheath and controls the ablation catheter position and energy delivery. In some embodiments, the handle comprises a reservoir to temporarily or permanently store the ablation medium. In some embodiments, the handle is designed for a right-handed person or a left-handed person to operate the catheter device efficiently and effectively. In some embodiments, the handle comprises an elongated handle housing having a proximal end, a distal end, and a longitudinal axis extending from the proximal end to the distal end. In some embodiments, the handle housing encloses the reservoir.

In some embodiments, accessing the gallbladder with the catheter device provided herein is achieved through a percutaneous approach. In some embodiments, the device access sheath 6 of the catheter device accesses the gallbladder 2 through a transhepatic, percutaneous approach using ultrasound guidance, as seen in FIG. 1A. In some embodiments, the device access sheath 6 of the catheter device accesses the gallbladder 2 through a subhepatic, percutaneous approach using ultrasound guidance, as seen in FIG. 1B. In some embodiments, the percutaneous approach is similar to the method used to place a cholecystostomy drain. In some embodiments, the catheter device provided herein accesses the gallbladder 2 endoscopically, as shown in FIG. 1C. In some embodiments, the device access sheath 6 of the catheter device accesses the gallbladder 2 utilizing native anatomy by creating a transmural stoma connecting the inner lumen of the gallbladder to the lumen of the small bowel, as shown in FIG. 1C. In some embodiments, percutaneous access is gained using a hollow bore needle, whereby a guidewire is placed through the needle to create a tract to the desired access location (e.g., a cystic duct, a gallbladder, or a combination thereof). In some embodiments, the device access sheath and the ablation catheter are configured with a concentric lumen to enable a guidewire to pass through. In some embodiments, the device access sheath and the ablation catheter are configured with a non-concentric lumen to enable a guidewire to pass through.

In some embodiments, the catheter device provided herein is a device for gallbladder defunctionalization. In some embodiments, once the catheter device accesses the gallbladder by its device access sheath, the content of the gallbladder is removed, similar to a cholecystostomy drain procedure. In some embodiments, the content of the gallbladder is removed in a prior procedure before the catheter device accesses the gallbladder by its device access sheath. In some embodiments, once the gallbladder 2 is accessed by the catheter device 4, the device is delivered into the cystic duct, whereby the distal end of the catheter occludes the cystic duct and prevents bile from entering the gallbladder. Next, in some embodiments, an ablation delivery system, located within the main body of the gallbladder, is deployed to defunctionalize the mucosal layer of gallbladder. The device is removed, and an integrated drainage catheter (not shown in the figures) is left in place while healing occurs over the next few weeks. In some embodiments, the device access sheath is left in place and act as a drainage catheter while healing occurs over the next few weeks.

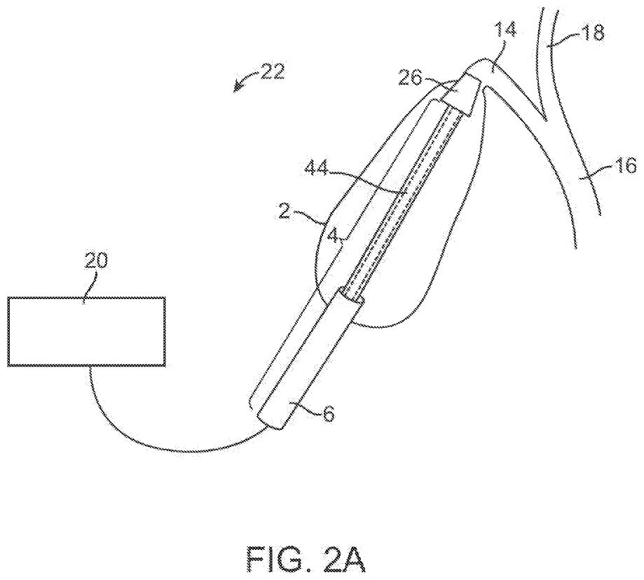

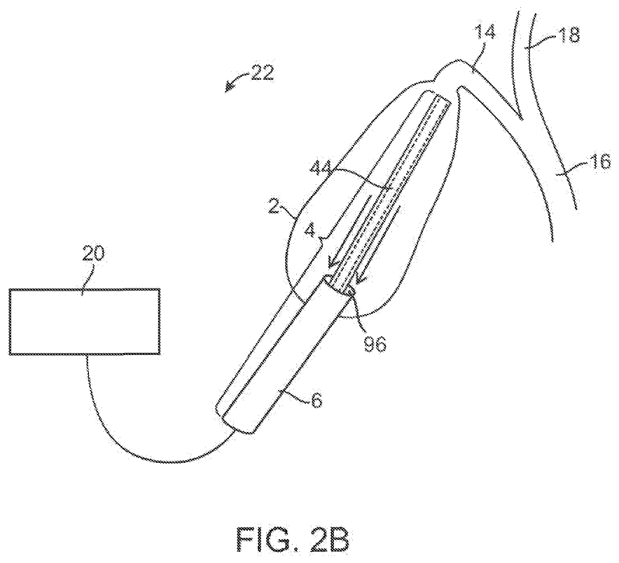

As shown in FIG. 2A, in some embodiments, the ablation delivery system 22 comprises a catheter device 4, an extracorporeal control unit 20, and a cystic duct occluder 26. In some embodiments, the catheter device 4 comprises a catheter and a device access sheath 6. In some embodiments, the catheter comprises a fenestrated nozzle 44, as shown in FIG. 2A. In some embodiments, the fenestrated nozzle 44 is an area of the catheter that comprises a plurality of fenestrations. In some embodiments, the catheter device 4 is deployed to defunctionalize the mucosal layer of gallbladder. In some embodiments, the cystic duct occluder 26 occludes the cystic duct and prevents bile from entering the gallbladder. In some embodiments, the cystic duct occluder 26 is a plug. In some embodiments, the cystic duct occluder 26 is an ablation medium, an ablation balloon, a radiofrequency (RF) ablater, or any combination thereof.

In some embodiments, the ablation delivery system 22 does not comprise a cystic duct occluder 26, as seen in FIG. 2B. In some embodiments, the device access sheath 6 comprises a device access sheath lumen 96 that has a diameter that is greater than the diameter of the catheter, as shown in FIG. 2B. In some embodiments, the device access sheath 6 is a passageway or a channel which is used to collect an ablation medium, to passively evacuate an ablation medium, to actively evacuate an ablation medium, or any combination thereof. In some embodiments, the device access sheath lumen 96 having a diameter that is greater than the diameter of the catheter allows for the collection of an ablation medium, for the passive evacuation of an ablation medium, for the active evacuation of an ablation medium, or any combination thereof by serving as a conduit or channel in which the ablation medium located in the gallbladder can flow through in the direction of the arrows shown in FIG. 2B and exit the gallbladder.

In some embodiments, the device access sheath 6 encloses one catheter. In some embodiments, the device access sheath 6 encloses two catheters. In some embodiments, the device access sheath 6 encloses three catheters. In some embodiments, the device access sheath lumen 96 has a diameter sufficiently large to accommodate one or more catheters. In some embodiments, the device access sheath lumen 96 has a diameter sufficiently large to accommodate two catheters. In some embodiments, the device access sheath lumen 96 has a diameter sufficiently large to accommodate three catheters. In some embodiments, the device access sheath lumen 96 has a diameter sufficiently large to accommodate about 1 catheter to about 10 catheters. In some embodiments, the device access sheath lumen 96 has a diameter sufficiently large to accommodate about 1 catheter to about 2 catheters, about 1 catheter to about 3 catheters, about 1 catheter to about 4 catheters, about 1 catheter to about 5 catheters, about 1 catheter to about 6 catheters, about 1 catheter to about 7 catheters, about 1 catheter to about 8 catheters, about 1 catheter to about 9 catheters, about 1 catheter to about 10 catheters, about 2 catheters to about 3 catheters, about 2 catheters to about 4 catheters, about 2 catheters to about 5 catheters, about 2 catheters to about 6 catheters, about 2 catheters to about 7 catheters, about 2 catheters to about 8 catheters, about 2 catheters to about 9 catheters, about 2 catheters to about 10 catheters, about 3 catheters to about 4 catheters, about 3 catheters to about 5 catheters, about 3 catheters to about 6 catheters, about 3 catheters to about 7 catheters, about 3 catheters to about 8 catheters, about 3 catheters to about 9 catheters, about 3 catheters to about 10 catheters, about 4 catheters to about 5 catheters, about 4 catheters to about 6 catheters, about 4 catheters to about 7 catheters, about 4 catheters to about 8 catheters, about 4 catheters to about 9 catheters, about 4 catheters to about 10 catheters, about 5 catheters to about 6 catheters, about 5 catheters to about 7 catheters, about 5 catheters to about 8 catheters, about 5 catheters to about 9 catheters, about 5 catheters to about 10 catheters, about 6 catheters to about 7 catheters, about 6 catheters to about 8 catheters, about 6 catheters to about 9 catheters, about 6 catheters to about 10 catheters, about 7 catheters to about 8 catheters, about 7 catheters to about 9 catheters, about 7 catheters to about 10 catheters, about 8 catheters to about 9 catheters, about 8 catheters to about 10 catheters, or about 9 catheters to about 10 catheters. In some embodiments, the device access sheath lumen 96 has a diameter sufficiently large to accommodate about 1 catheter, about 2 catheters, about 3 catheters, about 4 catheters, about 5 catheters, about 6 catheters, about 7 catheters, about 8 catheters, about 9 catheters, or about 10 catheters. In some embodiments, the device access sheath lumen 96 has a diameter sufficiently large to accommodate at least about 1 catheter, about 2 catheters, about 3 catheters, about 4 catheters, about 5 catheters, about 6 catheters, about 7 catheters, about 8 catheters, or about 9 catheters. In some embodiments, the device access sheath lumen 96 has a diameter sufficiently large to accommodate at most about 2 catheters, about 3 catheters, about 4 catheters, about 5 catheters, about 6 catheters, about 7 catheters, about 8 catheters, about 9 catheters, or about 10 catheters.

In some embodiments, as shown in FIGS. 2A-2B, the ablation delivery system 22 comprises an extracorporeal control unit 20. In some embodiments, the extracorporeal control unit 20 is operatively connected to the catheter device 4. In some embodiments, the extracorporeal control unit 20 is operatively connected to the device access sheath 6. In some embodiments, the extracorporeal control unit 20 is operatively connected to the cystic duct occluder 26. In some embodiments, the extracorporeal control unit 20 is part of the computer control system of the catheter device 4. In some embodiments, the extracorporeal control unit 20 is a handle (not shown in figures). In some embodiments, the ablation delivery system 22 comprises a temperature sensor, a pressure sensor, or a combination thereof. In some embodiments, the extracorporeal control unit 20 controls any sensor of the catheter device 4 or the cystic duct occluder (e.g., a pressure sensor, a temperature sensor, or any combination thereof). In some embodiments, the extracorporeal control unit 20 controls any mechanical movement of the catheter device 4 (e.g., deployment, retraction of a catheter, or any combination thereof). In some embodiments, the extracorporeal control unit 20 controls the passive or active evacuation of any fluid, gas, or any combination thereof through a sheath, catheter, or any combination thereof of the catheter device 4 (e.g., inflation of an ablation balloon). In some embodiments, the extracorporeal control unit interfaces with the ablation source and regulates or monitors the ablation medium supply pressure, the ablation medium flow rate, or a combination thereof.

In some embodiments, the extracorporeal control unit 20 comprises a connection for a visual output for a user. In some embodiments, the visual output is a digital output or an analog output. In some embodiments, the visual output comprises a temperature measurement, a pressure measurement, or a combination thereof.

In some embodiments, the catheter device comprises a display screen (not shown in the figures). In some embodiments, the display screen is operatively connected to the extracorporeal control unit 20. In some embodiments, the extracorporeal control unit 20 comprises a display screen. In some embodiments, the display screen provides visual information to a user. In some embodiments, the display screen is operatively connected to the catheter device. In some embodiments, the display screen displays a sensor reading to a user. In some embodiments, the display screen displays a sensor reading to a user in real time. In some embodiments, the display screen displays a temperature sensor reading to a user in real time. In some embodiments, the display screen displays a pressure sensor reading to a user in real time.

In some embodiments, the display screen is a computer screen, a mobile device screen, or a portable device screen. In some embodiments, the display screen is a tablet screen. In some embodiments, the display screen is a mobile phone screen. In some embodiments, the display screen is a touch screen. In some embodiments, the display screen is a liquid crystal display (LCD). In further embodiments, the display screen is a thin film transistor liquid crystal display (TFT-LCD). In some embodiments, the display screen is an organic light emitting diode (OLED) display. In various further embodiments, an OLED display is a passive-matrix OLED (PMOLED) or an active-matrix OLED (AMOLED) display. In some embodiments, the display screen is a plasma display. In some embodiments, the display screen is a video projector. In still further embodiments, the display screen is a combination of display screen types such as those disclosed herein. In some embodiments, the display screen is a full color display. In some embodiments, the display screen is a monochromatic display.

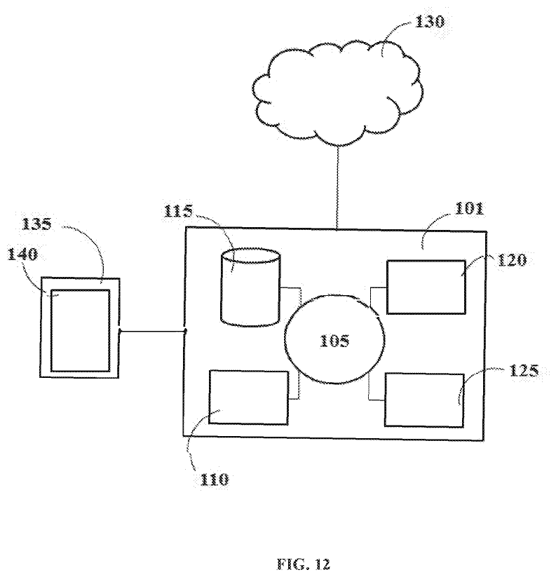

In some embodiments, the catheter device comprises a user interface, as illustrated in FIG. 12. In some embodiments, the user interface is operatively connected to the catheter device. In some embodiments, the user interface is operatively connected to the extracorporeal control unit 20. In some embodiments, the user interface is a function of the extracorporeal control unit 20. In some embodiments, the user interface allows a user to control an ablation source (e.g., an ablation medium supply pressure and an ablation medium supply flow rate). In some embodiments, the user interface allows a user to control a cryogen supply pressure while performing a gallbladder defunctionalization procedure using the devices disclosed herein. In some embodiments, the user interface allows a user to control a cryogen supply pressure while performing a cystic duct occlusion procedure using the devices disclosed herein. In some embodiments, the user interface allows a user to control a cryogen supply flow rate while performing a gallbladder defunctionalization procedure using the devices disclosed herein. In some embodiments, the user interface allows a user to control a cryogen supply flow rate while performing a cystic duct occlusion procedure using the devices disclosed herein. For example, in some embodiments, the user controls the supply pressure of cryogen being delivered to the lumen of a gallbladder or a cystic duct using the catheter devices disclosed herein. In yet another example, the user controls the supply flow rate of a cryogen being delivered to the lumen of a gallbladder or a cystic duct using the catheter devices disclosed herein.

### Device Access Sheath

In some embodiments, the catheter device 4 comprises a device access sheath 6. In some embodiments, the device access sheath 6 envelops, covers, encases, or surrounds one or more catheters to be inserted into a tissue of an individual in need thereof. In some embodiments, the tissue is a gallbladder, a liver, adipose tissue, skin, pancreas, stomach, spleen, small intestine, large intestine, a blood vessel, or any combination thereof. In some embodiments, the device access sheath 6 comprises at least one lumen. In some embodiments, one or more catheters to be inserted into a tissue of an individual in need thereof are placed within the at least one lumen of the device access sheath 6.

In some embodiments, the device access sheath 6 provides access to the gallbladder lumen and allows for additional tools, procedures, or any combination thereof to be performed throughout. In some embodiments, the device access sheath 6 acts as a channel to drain an ablation medium from the lumen of a gallbladder. In some embodiments, the device access sheath 6 provides access to drain an ablation medium from the lumen of a gallbladder. In some embodiments, the ablation medium is drained or passively evacuated from the lumen of a gallbladder via the device access sheath 6. In some embodiments, the ablation medium is passively evacuated from the lumen of a gallbladder by having the ablation medium exit the lumen of the gallbladder, enter the lumen of the device access sheath 6, flow away from the gallbladder, and collected extracorporeally (by a fluid collection system, for example). In some embodiments, the ablation medium is actively evacuated from the lumen of a gallbladder via the device access sheath 6. In some embodiments, the ablation medium is actively evacuated from the lumen of a gallbladder via the device access sheath 6 by operatively connecting a vacuum source to the device access sheath 6.

In some embodiments, the device access sheath 6 comprises one or more catheters. In some embodiments, the device access sheath 6 comprises drainage catheter that is used to passively remove or evacuate an ablation medium from the lumen of a gallbladder. For example, in some embodiments, the ablation medium is passively evacuated from the lumen of a gallbladder via the device access sheath 6 by having the ablation medium exit the lumen of the gallbladder, enter the lumen of the drainage catheter, flow away from the gallbladder, and collected extracorporeally (by a fluid collection system, for example). In some embodiments, the device access sheath 6 comprises a drainage catheter that is used to actively remove or evacuate an ablation medium from the lumen of a gallbladder. For example, in some embodiments, the ablation medium is actively evacuated from the lumen of a gallbladder via the device access sheath 6 by operatively connecting a vacuum source to a drainage catheter that is placed within the lumen of the device access sheath 6.

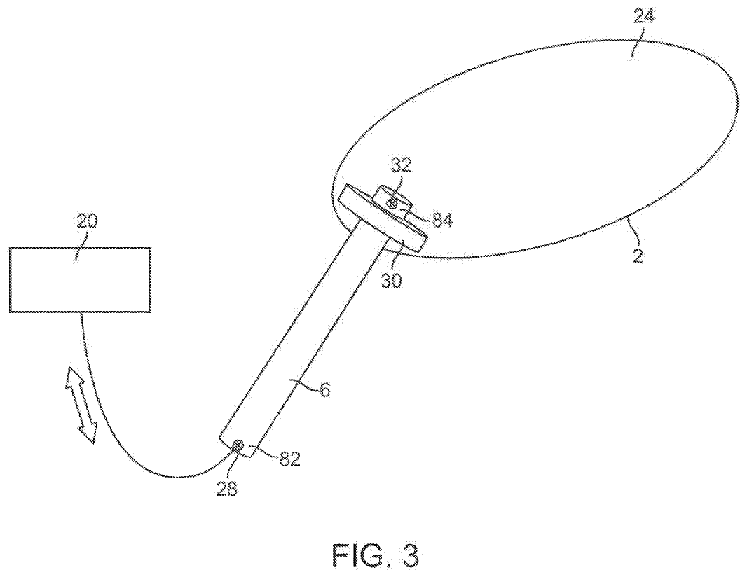

In some embodiments, the device access sheath is a tube having a distal end 84, a proximal end 82, and at least one lumen (not shown in the figures), as shown in FIG. 3. In some embodiments, the distal end 84 of the device access sheath 6 is placed within the gallbladder lumen 24. In some embodiments, the distal end 84 of the device access sheath 6 has a deployable geometry that prevents dislodgement and creates a seal 30 between the access lumen and the gallbladder 2, as seen in FIG. 3. In some embodiments, the seal 30 has a shape that resembles the shape of a Malecot catheter. In some embodiments, the seal 30 has a geometry that is stressed upon delivery, elongating or increasing its shape, but then returns to its original shape (i.e., its resting state) after delivery. In some embodiments, the resting state of the seal comprises the seal 30 having a diameter that is larger than an access opening (e.g., the access opening in a gallbladder of a patient). In some embodiments, the seal 30 is a plastic seal. In some embodiments, the seal 30 is a rubber seal. In some embodiments, the seal 30 is a lip or a ring enveloping the circumference of the device access sheath 6 at its distal end. In some embodiments, the seal 30 is composed of a shape memory material. In some embodiments, the seal 30 is composed of a polymeric material. Non-limiting examples of polymeric materials include nylon, polyvinyl chloride (PVC), urethane, and silicone. In some embodiments, the seal 30 comprises a diameter that is larger than the diameter of the device access sheath 6. In some embodiments, the seal 30 comprises a diameter that is about 1.5 times larger than the diameter of the device access sheath 6. In some embodiments, the seal 30 comprises a diameter that is 2 times larger than the diameter of the device access sheath 6. In some embodiments, the seal 30 comprises a diameter that is about 3 times larger than the diameter of the device access sheath 6. In some embodiments, the seal 30 a seal extends along the circumference of the access sheath at the distal end of the access sheath.

In some embodiments, the seal comprises a deployable nitinol geometry that expands to a final conformation larger than that of the access sheath diameter. In some embodiments, the seal is a deformable polymer structure that expands to a final conformation larger than that of the access sheath diameter, similar to Malecot catheter devices. In some embodiments, the proximal end 82 of the device access sheath 6 has a skin interface, which allows for adhesive or mechanical securement of the lumen to the patient's skin as seen in FIG. 3. In some embodiments, the device access sheath 6 interfaces with existing drainage tubing and collection bags for fluid containment. In some embodiments, the device access sheath 6 is optionally placed with a guidewire. In some embodiments, the seal is sufficiently rigid to allow the user to pull traction on the access sheath, whereby opposing the gallbladder tissue to that of surrounding organs, such as the liver, the abdominal wall, or a combination thereof.

In some embodiments, the deployable geometry, located on the distal end 84 of the device access sheath 6, comprises a balloon. In some embodiments, the balloon is an inflatable balloon. In some embodiments, the balloon is a compliant balloon. In some embodiments, the compliant balloon expands as internal pressure increases. In some embodiments, the compliant balloon is used to occlude a tissue, to expand a tissue, to hold the catheter device in position, or any combination thereof. In some embodiments, the balloon is a semi-compliant balloon. In some embodiments, the balloon is a non-compliant balloon. In some embodiments the semi-compliant balloon and the non-compliant balloon expand to a specific size or size range, even as internal pressure increases. In some embodiments the semi-compliant balloon and the non-compliant balloon are used to apply force or occlude. In some embodiments, the balloon can inflate radially to achieve a ring conformation, whereby the diameter of the balloon is larger than the diameter of the access sheath.

In some embodiments, the diameter of the balloon is about 1.1 times to about 5 times larger than the diameter of the device access sheath. In some embodiments, the diameter of the balloon is about 1.1 times to about 1.2 times, about 1.1 times to about 1.3 times, about 1.1 times to about 1.4 times, about 1.1 times to about 1.5 times, about 1.1 times to about 1.6 times, about 1.1 times to about 1.7 times, about 1.1 times to about 1.8 times, about 1.1 times to about 1.9 times, about 1.1 times to about 2 times, about 1.1 times to about 3 times, about 1.1 times to about 5 times, about 1.2 times to about 1.3 times, about 1.2 times to about 1.4 times, about 1.2 times to about 1.5 times, about 1.2 times to about 1.6 times, about 1.2 times to about 1.7 times, about 1.2 times to about 1.8 times, about 1.2 times to about 1.9 times, about 1.2 times to about 2 times, about 1.2 times to about 3 times, about 1.2 times to about 5 times, about 1.3 times to about 1.4 times, about 1.3 times to about 1.5 times, about 1.3 times to about 1.6 times, about 1.3 times to about 1.7 times, about 1.3 times to about 1.8 times, about 1.3 times to about 1.9 times, about 1.3 times to about 2 times, about 1.3 times to about 3 times, about 1.3 times to about 5 times, about 1.4 times to about 1.5 times, about 1.4 times to about 1.6 times, about 1.4 times to about 1.7 times, about 1.4 times to about 1.8 times, about 1.4 times to about 1.9 times, about 1.4 times to about 2 times, about 1.4 times to about 3 times, about 1.4 times to about 5 times, about 1.5 times to about 1.6 times, about 1.5 times to about 1.7 times, about 1.5 times to about 1.8 times, about 1.5 times to about 1.9 times, about 1.5 times to about 2 times, about 1.5 times to about 3 times, about 1.5 times to about 5 times, about 1.6 times to about 1.7 times, about 1.6 times to about 1.8 times, about 1.6 times to about 1.9 times, about 1.6 times to about 2 times, about 1.6 times to about 3 times, about 1.6 times to about 5 times, about 1.7 times to about 1.8 times, about 1.7 times to about 1.9 times, about 1.7 times to about 2 times, about 1.7 times to about 3 times, about 1.7 times to about 5 times, about 1.8 times to about 1.9 times, about 1.8 times to about 2 times, about 1.8 times to about 3 times, about 1.8 times to about 5 times, about 1.9 times to about 2 times, about 1.9 times to about 3 times, about 1.9 times to about 5 times, about 2 times to about 3 times, about 2 times to about 5 times, or about 3 times to about 5 times larger than the diameter of the device access sheath. In some embodiments, the diameter of the balloon is about 1.1 times, about 1.2 times, about 1.3 times, about 1.4 times, about 1.5 times, about 1.6 times, about 1.7 times, about 1.8 times, about 1.9 times, about 2 times, about 3 times, or about 5 times larger than the diameter of the device access sheath. In some embodiments, the diameter of the balloon is at least about 1.1 times, about 1.2 times, about 1.3 times, about 1.4 times, about 1.5 times, about 1.6 times, about 1.7 times, about 1.8 times, about 1.9 times, about 2 times, or about 3 times larger than the diameter of the device access sheath. In some embodiments, the diameter of the balloon is at most about 1.2 times, about 1.3 times, about 1.4 times, about 1.5 times, about 1.6 times, about 1.7 times, about 1.8 times, about 1.9 times, about 2 times, about 3 times, or about 5 times larger than the diameter of the device access sheath.

In some embodiments, the inflatable balloon is composed of a non-compliant, a semi-compliant, or a compliant material. Non-limiting examples of a non-compliant material include polyethylene terephthalate (PET), polyester, and nylon. Non-limiting examples of a semi-compliant material include polyether block amide (PEBA) and high durometer polyurethane. Non-limiting examples of a compliant material include silicone, latex, liquid silicone rubber, polyolefin copolymer (POC), and polyurethane.

In some embodiments, the balloon has a compliance of at least about 0% to about 500%. In some embodiments, the non-compliant balloon has a compliance ranging from about 0% to about 7%. In some embodiments, the non-compliant balloon has a compliance ranging from about 0% to about 1%, about 0% to about 2%, about 0% to about 3%, about 0% to about 4%, about 0% to about 5%, about 0% to about 6%, about 0% to about 7%, about 1% to about 2%, about 1% to about 3%, about 1% to about 4%, about 1% to about 5%, about 1% to about 6%, about 1% to about 7%, about 2% to about 3%, about 2% to about 4%, about 2% to about 5%, about 2% to about 6%, about 2% to about 7%, about 3% to about 4%, about 3% to about 5%, about 3% to about 6%, about 3% to about 7%, about 4% to about 5%, about 4% to about 6%, about 4% to about 7%, about 5% to about 6%, about 5% to about 7%, or about 6% to about 7%. In some embodiments, the non-compliant balloon has a compliance ranging from about 0%, about 1%, about 2%, about 3%, about 4%, about 5%, about 6%, or about 7%. In some embodiments, the non-compliant balloon has a compliance ranging from at least about 0%, about 1%, about 2%, about 3%, about 4%, about 5%, or about 6%. In some embodiments, the non-compliant balloon has a compliance ranging from at most about 1%, about 2%, about 3%, about 4%, about 5%, about 6%, or about 7%.

In some embodiments, the semi-compliant balloon has a compliance ranging from about 5% to about 10%. In some embodiments, the semi-compliant balloon has a compliance ranging from about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10%, or about 9% to about 10%. In some embodiments, the semi-compliant balloon has a compliance ranging from about 5%, about 6%, about 7%, about 8%, about 9%, or about 10%. In some embodiments, the semi-compliant balloon has a compliance ranging from at least about 5%, about 6%, about 7%, about 8%, or about 9%. In some embodiments, the semi-compliant balloon has a compliance ranging from at most about 6%, about 7%, about 8%, about 9%, or about 10%.

In some embodiments, the compliant balloon has a compliance ranging from about 10% to about 500%. In some embodiments, the compliant balloon has a compliance ranging from about 10% to about 50%, about 10% to about 100%, about 10% to about 150%, about 10% to about 200%, about 10% to about 250%, about 10% to about 300%, about 10% to about 350%, about 10% to about 400%, about 10% to about 450%, about 10% to about 500%, about 50% to about 100%, about 50% to about 150%, about 50% to about 200%, about 50% to about 250%, about 50% to about 300%, about 50% to about 350%, about 50% to about 400%, about 50% to about 450%, about 50% to about 500%, about 100% to about 150%, about 100% to about 200%, about 100% to about 250%, about 100% to about 300%, about 100% to about 350%, about 100% to about 400%, about 100% to about 450%, about 100% to about 500%, about 150% to about 200%, about 150% to about 250%, about 150% to about 300%, about 150% to about 350%, about 150% to about 400%, about 150% to about 450%, about 150% to about 500%, about 200% to about 250%, about 200% to about 300%, about 200% to about 350%, about 200% to about 400%, about 200% to about 450%, about 200% to about 500%, about 250% to about 300%, about 250% to about 350%, about 250% to about 400%, about 250% to about 450%, about 250% to about 500%, about 300% to about 350%, about 300% to about 400%, about 300% to about 450%, about 300% to about 500%, about 350% to about 400%, about 350% to about 450%, about 350% to about 500%, about 400% to about 450%, about 400% to about 500%, or about 450% to about 500%. In some embodiments, the compliant balloon has a compliance ranging from about 10%, about 50%, about 100%, about 150%, about 200%, about 250%, about 300%, about 350%, about 400%, about 450%, or about 500%. In some embodiments, the compliant balloon has a compliance ranging from at least about 10%, about 50%, about 100%, about 150%, about 200%, about 250%, about 300%, about 350%, about 400%, or about 450%. In some embodiments, the compliant balloon has a compliance ranging from at most about 50%, about 100%, about 150%, about 200%, about 250%, about 300%, about 350%, about 400%, about 450%, or about 500%.

In some embodiments, the inflatable balloon is filled with a gas, such as carbon dioxide (CO₂), to achieve its final conformation. In some embodiments, the balloon is filled with a liquid, such as a saline solution, a dextrose solution, or any combination thereof, to achieve its final conformation.

In some embodiments, the device access sheath comprises a distal end 84, a proximal end 82, and an elongated body therebetween. In some embodiments, the device access sheath 6 comprises a catheter with multiple lumens. In some embodiments, the device access sheath 6 comprises a catheter with multiple lumens at the distal end of the catheter. In some embodiments, the catheter comprising one or more lumens is inserted into the lumen of the gallbladder. In some embodiments, the device access sheath 6 comprises multiple catheters covered by an outer sheath. In some embodiments, the device access sheath 6 comprises multiple catheters that are configured to move independently of each other. In some embodiments, the device access sheath 6 is optionally used with a guidewire, a dilator, or a combination thereof in order to gain access to a desired location (e.g., a gallbladder lumen).

In some embodiments, the device access sheath provides active removal of debris from the gallbladder lumen. In some instances, the debris, actively removed by the catheter device of the present disclosure, includes mammalian cells. In some instances, the debris includes components of mammalian cells. In some instances, the debris includes bile. In some embodiments, the debris comprises cholesterol. In some embodiments, the debris comprises bacteria. In some embodiments, the debris comprises infected tissue. In some embodiments, the mammalian cells originate from a tissue in the individual in need thereof. In some embodiments, the tissue is a gallbladder, a liver, adipose tissue, skin, pancreas, stomach, spleen, small intestine, large intestine, a blood vessel, or any combination thereof. In some instances, said debris includes gallstones. In some instances, the debris includes parts or fragments of gallstones. In some instances, the debris includes saline. In some instances, the debris includes a lavage medium. In some instances, the debris includes an ablation medium. In some instances, the debris includes gas.

In some embodiments, the catheter device provides active removal of debris from the gallbladder lumen by applying a controlled amount of vacuum to the proximal end 82 of the device access sheath 6. In some instances, the controlled amount of vacuum that is applied is translated to the distal end 82 via a hollow bore in the catheter that is located within the lumen of the device access sheath 6. In some instances, the controlled amount of vacuum is applied is to the proximal end 82 of the device access sheath 6 in the absence of a catheter located within the lumen of the device access sheath 6. In some instances, the vacuum is applied to the gallbladder lumen via fenestrations in the catheter body. In some instances, the fenestrations are at the distal end. In some instances, the fenestrations are anywhere else along the length of the catheter.

As shown in in FIG. 3, in some embodiments, the device access sheath 6 comprises at least one temperature sensor 32 to detect or measure the temperature within the gallbladder lumen. In some embodiments, the temperature sensor(s) 32 is located at the distal end 84 of the device access sheath 6, within the gallbladder lumen (when device is inserted in an individual in need thereof). In some embodiments, the temperature sensor 32 interfaces with the extracorporeal control unit to display intraluminal temperature values. In some embodiments, the temperature sensor 32 is operatively connected to the extracorporeal control unit 20, as shown in FIG. 3. In some embodiments, the temperature sensor 32 is located at the proximal end 82 of the device access sheath 6. In some embodiments, the temperature sensor 32 is located anywhere along the elongated body of the device access sheath 6 between the distal end 84 and the proximal end 82.

In some embodiments, the temperature sensor 32 is configured to detect a temperature of the ablation medium in the gallbladder, of a fluid in the gallbladder, or a combination thereof. In some embodiments, the temperature sensor 32 is located at the distal end of the system, in fluid connection with a lumen of the gallbladder, when in use. In some embodiments, the temperature sensor 32 is located at the distal end of the device access sheath 4, in fluid connection with a lumen of the gallbladder, when in use. In some embodiments, the temperature sensor 32 is located at the distal end of the catheter 66, in fluid connection with a lumen of the gallbladder, when in use. In some embodiments, the temperature sensor 32 is located on the body of the device access sheath 4, in fluid connection with a lumen of the gallbladder, when in use. In some embodiments, the temperature sensor 32 is located on the body of the catheter 66, in fluid connection with a lumen of the gallbladder, when in use. In some embodiments, the temperature sensor 32 is located within a lumen of the gallbladder, when in use. In some embodiments, the temperature sensor 32 is part of a confirmation circuit that provides a user with an intraluminal temperature of the gallbladder, when in use. In some embodiments, the confirmation circuit is part of the extracorporeal control unit 20 or of the computing system of the catheter device system. In some embodiments, the the temperature sensor 32 is an optional component of the catheter systems provided herein.

In some embodiments, the catheter device 4 contains at least one pressure sensor 28 to detect or measure the pressure within the gallbladder lumen. In some embodiments, the pressure sensor(s) 28 is located on the proximal end 82 of the device access sheath 6, as shown in FIG. 3. In some embodiments, the pressure sensor 28 is located at the distal end 84 of the device access sheath 6. In some embodiments, the pressure sensor 28 is located anywhere along the elongated body of the device access sheath 6 between the distal end 84 and the proximal end 82. In some embodiments, the elongated body of the access sheath translates intraluminal gallbladder pressures to a sensor located on the proximal end of the access sheath. In some embodiments, the pressure sensor(s) is on the distal end of the device access sheath, within the gallbladder lumen. In some embodiments, the pressure measurement sensor interfaces with the extracorporeal control unit to display pressure values. In some embodiments, the pressure sensor 28 is operatively connected to the extracorporeal control unit 20, as shown in FIG. 3.

In some embodiments, the pressure sensor 28 is a pressure transducer. In some embodiments, the pressure sensor 28 is a guidewire pressure transducer. In some embodiments, the pressure sensor 28 is a catheter pressure transducer. In some embodiments, the pressure sensor 28 is a strain gauge transducer. In some embodiments, the pressure sensor 28 is a diaphragm displacement sensor. In some embodiments, the pressure sensor 28 is an optical fiber pressure sensor.

In some embodiments, the active evacuation disclosed herein is controlled by a feedback loop. In some embodiments, the access sheath is coupled to an active evacuation mechanism to prevent pressure build-up in the gallbladder lumen via a close loop feedback system. In some embodiments, the access sheath is coupled to a passive evacuation system to prevent pressure from building up in the gallbladder lumen In some instances, the feedback loop consists of a process whereby the active evacuation is automatically applied to the system when the pressure within the gallbladder lumen (as detected by the pressure sensor 28 described above) exceeds a certain threshold pressure. In some instances, the pressure within the gallbladder lumen is detected by the pressure sensor 28 from the present disclosure. In some embodiments, the feedback loop prevents the gallbladder lumen from exceeding a set threshold pressure. In some embodiments, the feedback loop and active evacuation compensate for increased gas or liquid volume within the gallbladder lumen, due to the introduction of ablation medium such as nitrous oxide or steam

In some embodiments, the access sheath is coupled to an active evacuator to prevent pressure from building up in the gallbladder lumen. In some embodiments, the active evacuator is a vacuum pump that generates a suction force. In some embodiments, the evacuation of the ablation medium is an active evacuation pulling negative pressure through the lumen of the access sheath. In some embodiments, the active evacuator pulls negative pressure through the lumen of the access sheath. In some embodiments, the active evacuator pulls negative pressure through the lumen of the access sheath thereby removing ablation medium from the gallbladder lumen, a lumen of an ablation balloon, a lumen of a fenestrated ablation balloon, a lumen of a catheter, a lumen of the device access sheath, or any combination thereof. In some embodiments, the feedback loop allows for insufflation of the gallbladder lumen without exceeding a threshold pressure. In some embodiments, the threshold pressure ranges from about 0 millimeters of mercury (mmHg) to about 500 mmHg. In some embodiments, the threshold pressure ranges from about 30 to about 40 mmHg. In some embodiments, the threshold pressure ranges from about 0 to about 100 mmHg. In some embodiments, the threshold pressure ranges from about 5 millimeters of mercury (mmHg) to about 500 mmHg. In some embodiments, the threshold pressure ranges from about 5 mmHg to about 10 mmHg, about 5 mmHg to about 50 mmHg, about 5 mmHg to about 75 mmHg, about 5 mmHg to about 100 mmHg, about 5 mmHg to about 150 mmHg, about 5 mmHg to about 200 mmHg, about 5 mmHg to about 250 mmHg, about 5 mmHg to about 300 mmHg, about 5 mmHg to about 350 mmHg, about 5 mmHg to about 400 mmHg, about 5 mmHg to about 500 mmHg, about 10 mmHg to about 50 mmHg, about 10 mmHg to about 75 mmHg, about 10 mmHg to about 100 mmHg, about 10 mmHg to about 150 mmHg, about 10 mmHg to about 200 mmHg, about 10 mmHg to about 250 mmHg, about 10 mmHg to about 300 mmHg, about 10 mmHg to about 350 mmHg, about 10 mmHg to about 400 mmHg, about 10 mmHg to about 500 mmHg, about 50 mmHg to about 75 mmHg, about 50 mmHg to about 100 mmHg, about 50 mmHg to about 150 mmHg, about 50 mmHg to about 200 mmHg, about 50 mmHg to about 250 mmHg, about 50 mmHg to about 300 mmHg, about 50 mmHg to about 350 mmHg, about 50 mmHg to about 400 mmHg, about 50 mmHg to about 500 mmHg, about 75 mmHg to about 100 mmHg, about 75 mmHg to about 150 mmHg, about 75 mmHg to about 200 mmHg, about 75 mmHg to about 250 mmHg, about 75 mmHg to about 300 mmHg, about 75 mmHg to about 350 mmHg, about 75 mmHg to about 400 mmHg, about 75 mmHg to about 500 mmHg, about 100 mmHg to about 150 mmHg, about 100 mmHg to about 200 mmHg, about 100 mmHg to about 250 mmHg, about 100 mmHg to about 300 mmHg, about 100 mmHg to about 350 mmHg, about 100 mmHg to about 400 mmHg, about 100 mmHg to about 500 mmHg, about 150 mmHg to about 200 mmHg, about 150 mmHg to about 250 mmHg, about 150 mmHg to about 300 mmHg, about 150 mmHg to about 350 mmHg, about 150 mmHg to about 400 mmHg, about 150 mmHg to about 500 mmHg, about 200 mmHg to about 250 mmHg, about 200 mmHg to about 300 mmHg, about 200 mmHg to about 350 mmHg, about 200 mmHg to about 400 mmHg, about 200 mmHg to about 500 mmHg, about 250 mmHg to about 300 mmHg, about 250 mmHg to about 350 mmHg, about 250 mmHg to about 400 mmHg, about 250 mmHg to about 500 mmHg, about 300 mmHg to about 350 mmHg, about 300 mmHg to about 400 mmHg, about 300 mmHg to about 500 mmHg, about 350 mmHg to about 400 mmHg, about 350 mmHg to about 500 mmHg, or about 400 mmHg to about 500 mmHg. In some embodiments, the threshold pressure ranges from about 5 mmHg, about 10 mmHg, about 50 mmHg, about 75 mmHg, about 100 mmHg, about 150 mmHg, about 200 mmHg, about 250 mmHg, about 300 mmHg, about 350 mmHg, about 400 mmHg, or about 500 mmHg. In some embodiments, the threshold pressure ranges from at least about 5 mmHg, about 10 mmHg, about 50 mmHg, about 75 mmHg, about 100 mmHg, about 150 mmHg, about 200 mmHg, about 250 mmHg, about 300 mmHg, about 350 mmHg, or about 400 mmHg. In some embodiments, the threshold pressure ranges from at most about 10 mmHg, about 50 mmHg, about 75 mmHg, about 100 mmHg, about 150 mmHg, about 200 mmHg, about 250 mmHg, about 300 mmHg, about 350 mmHg, about 400 mmHg, or about 500 mmHg.

In some embodiments, a passive evacuation of the gallbladder is facilitated by pressure driven flow from the increase of pressure in the gallbladder lumen relative to atmospheric pressure. In some embodiments, a passive evacuation of the gallbladder is facilitated by a pressure gradient between the gallbladder lumen and atmospheric pressure. In some embodiments, the pressure of the gallbladder lumen is higher than a pressure in the lumen of the access device sheath. In some instances, the passive evacuation mechanism consists of a hollow bore lumen of sufficient size to allow for flow of gas/liquid to the atmosphere, without the assistance of suction. In some embodiments, the passive evacuation lumen contains a valve (not shown in the figures) to allow for a nominal pressure to build up within the gallbladder, while allowing for evacuation beyond a set mechanical threshold.

In some embodiments, the access sheath is coupled to a passive evacuator to prevent pressure from building up in the gallbladder lumen. In some embodiments, the passive evacuator is a vacuum pump that generates a suction force. In some embodiments, the evacuation of the ablation medium is an active evacuation pulling negative pressure through the lumen of the access sheath. In some embodiments, the evacuator pulls negative pressure through the lumen of the access sheath. In some embodiments, the evacuator pulls negative pressure through the lumen of the access sheath thereby removing ablation medium from the gallbladder lumen, a lumen of an ablation balloon, a lumen of a fenestrated ablation balloon, a lumen of a catheter, a lumen of the device access sheath, or any combination thereof.

In some embodiments, the nominal pressure ranges from about 30 mmHg to about 40 mmHg. In some embodiments, the nominal pressure ranges from about 5 mmHg to about 100 mmHg. In some embodiments, the nominal pressure ranges from about 5 mmHg to about 10 mmHg, about 5 mmHg to about 15 mmHg, about 5 mmHg to about 20 mmHg, about 5 mmHg to about 25 mmHg, about 5 mmHg to about 50 mmHg, about 5 mmHg to about 75 mmHg, about 5 mmHg to about 100 mmHg, about 10 mmHg to about 15 mmHg, about 10 mmHg to about 20 mmHg, about 10 mmHg to about 25 mmHg, about 10 mmHg to about 50 mmHg, about 10 mmHg to about 75 mmHg, about 10 mmHg to about 100 mmHg, about 15 mmHg to about 20 mmHg, about 15 mmHg to about 25 mmHg, about 15 mmHg to about 50 mmHg, about 15 mmHg to about 75 mmHg, about 15 mmHg to about 100 mmHg, about 20 mmHg to about 25 mmHg, about 20 mmHg to about 50 mmHg, about 20 mmHg to about 75 mmHg, about 20 mmHg to about 100 mmHg, about 25 mmHg to about 50 mmHg, about 25 mmHg to about 75 mmHg, about 25 mmHg to about 100 mmHg, about 50 mmHg to about 75 mmHg, about 50 mmHg to about 100 mmHg, or about 75 mmHg to about 100 mmHg. In some embodiments, the nominal pressure ranges from about 5 mmHg, about 10 mmHg, about 15 mmHg, about 20 mmHg, about 25 mmHg, about 50 mmHg, about 75 mmHg, or about 100 mmHg. In some embodiments, the nominal pressure ranges from at least about 5 mmHg, about 10 mmHg, about 15 mmHg, about 20 mmHg, about 25 mmHg, about 50 mmHg, or about 75 mmHg. In some embodiments, the nominal pressure ranges from at most about 10 mmHg, about 15 mmHg, about 20 mmHg, about 25 mmHg, about 50 mmHg, about 75 mmHg, or about 100 mmHg.

In some embodiments, the active evacuation disclosed herein is powered by existing aspiration systems. In some instances, a passive evacuation of an ablation medium is powered by the extracorporeal control unit 20. In some instances, an active evacuation of an ablation medium is powered by the extracorporeal control unit 20. In some embodiments, the active evacuation of the ablation medium comprises a vacuum pump. In some instances, the extracorporeal control unit 20 comprises a vacuum pump and a fluid collection system. In some embodiments, the catheter device disclosed herein is configured for connection to a standard hospital suction unit. In some instances, the catheter device is configured for connection to a wall suction system. In some instances, the catheter device is configured for connection to a portable suction unit. In some embodiments, the catheter device comprises a step-down regulator that is attached to or integrated into the device access sheath 6 to ensure a safe level of vacuum is introduced into the system. In some embodiments, the ablation medium evacuation flow rate is proportional to ablation medium supply flow rate.

In some embodiments, the material of the delivery access sheath 6 is flexible or semi-flexible, relatively non-distensible and is able to return substantially to its original configuration and orientation. In some embodiments, the material is biocompatible and is one or more medical grade materials.

In some embodiments, the catheter device provided herein comprises a guidewire. In some embodiments, the device access sheath 6 comprises a guidewire. In some embodiments, a catheter of the catheter device comprises a guidewire. In some embodiments, the distal tip of the guidewire, the distal end 84 of the access delivery sheath 6, or any combination thereof comprises a marker to aid tracking of the movement of the catheter-based device. In some embodiments, the distal end of the ablation catheter comprises at least one market to aid in the placement of device. In some embodiments, the marker is a radiopaque marker or a metal marker.

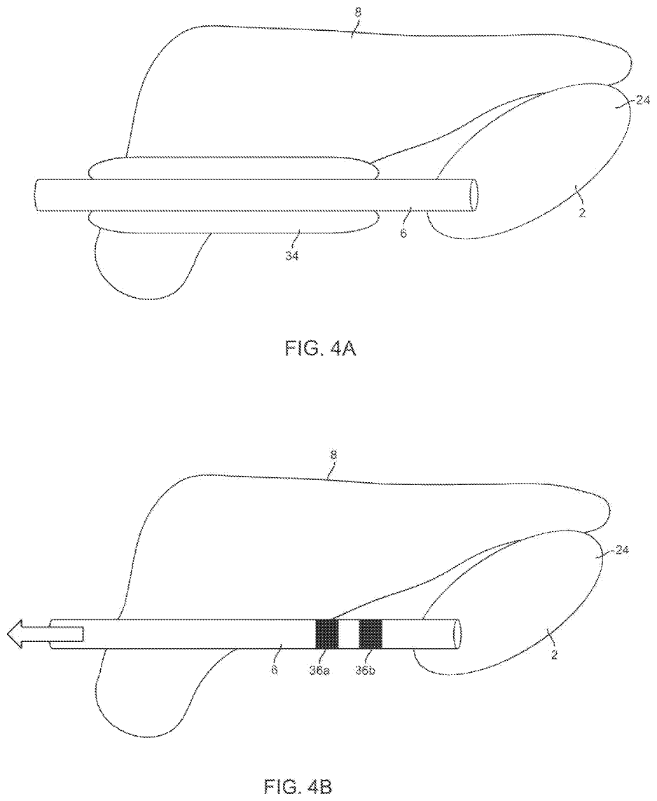

FIGS. 4A and 4B illustrate an exemplary device access sheaths that aid in minimizing or reducing blood loss of organs and tissues when accessing the gallbladder via a transhepatic route. In some embodiments, the device access sheath 6 minimizes or reduces blood loss, induces coagulation, alleviates or stops refractory bleeding, or any combination thereof of the liver 8 or tissues that are injured or damaged when accessing the gallbladder 2 via a transhepatic route by deploying a balloon tamponade 34, as shown in FIG. 4A. In some embodiments, the device access sheath 6 minimizes or reduces blood loss, induces coagulation, alleviates or stops refractory bleeding, or any combination thereof of the liver 8 or tissues that are injured or damaged when accessing the gallbladder 2 via a subhepatic route or any other suitable access route known by the skilled artisan by deploying a balloon tamponade 34. In some embodiments, the access delivery sheath 6 has a highly compliant outer covering of the elongated body. In some embodiments, the outer covering has a port on the extracorporeal end that facilitates filling of the space between the outer covering and outside of the access lumen with air or fluid (including, but not limited to water, saline, or contrast agent) to increase the capillary pressure at the tissue interface with the access lumen, establishing tamponade and promoting coagulation and sealing of the disrupted surface of tissue and organs such as the liver 8, as seen in FIG. 4A. In some embodiments, the device access sheath 6 comprises a balloon tamponade 34. In some embodiments, the balloon tamponade 34 promotes coagulation, alleviates or stops refractory bleeding from surrounding tissue, seals the disrupted surface of the surrounding tissue, or any combination thereof, as shown in FIG. 4A. In some embodiments, the balloon tamponade 34 is a compliant balloon, a non-compliant balloon, or a semi-compliant balloon. In some embodiments, the balloon tamponade 34 is an expandable balloon. In some embodiments, the balloon tamponade 34 is an inflatable balloon. In some embodiments, the balloon tamponade 34 surrounds the elongate body of the device access sheath 6. In some embodiments, the surface of the elongate body of the device access sheath 6 is in fluid communication with the interior lumen of the balloon tamponade 34. In some embodiments, a catheter comprises the balloon tamponade 34. In some embodiments, the balloon tamponade 34 is deployed from a catheter instead of being deployed from the device access sheath.

In some embodiments, the elongated body of the device access sheath 6 is coated with or embedded with a procoagulant material. In some embodiments, the surface of the balloon tamponade 34 is coated with or embedded with a procoagulant material. In some embodiments, the procoagulant material includes fibrin, thrombin, or other activating clotting factor. In some embodiments, contact between the tissue interface and the treated surface of the device access sheath 6 promotes clotting on the surface of the disrupted tissue. In some embodiments, contact between the tissue interface and the treated surface of the balloon tamponade 34 promotes clotting on the surface of the disrupted tissue.

In some embodiments, the device access sheath 6 minimizes or reduces blood loss, induces coagulation, alleviates or stops refractory bleeding, or any combination thereof of the liver 8 or tissues that are injured or damaged when accessing the gallbladder 2 via a transhepatic route by energizing and activating a pair of electrodes that ablate tissue, as shown in FIG. 4B. In some embodiments, the device access sheath 6 minimizes or reduces blood loss, induces coagulation, alleviates or stops refractory bleeding, or any combination thereof of the liver 8 or tissues that are injured or damaged when accessing the gallbladder 2 via a subhepatic route or any other suitable access route known by the skilled artisan by energizing and activating a pair of electrodes that ablate tissue. In some embodiments, the device access sheath 6 comprises a first electrode 36a and a second electrode 36b, as shown in FIG. 4B. In some embodiments, the first electrode 36a and the second electrode 36b are bipolar radiofrequency (RF) electrodes. In some embodiments, the first electrode 36a and the second electrode 36b are monopolar RF electrodes. In some embodiments, the first electrode 36a and the second electrode 36b are multipolar RF electrodes. In some embodiments the device access sheath 6 comprises at least one electrode to deliver an ablation energy. In some embodiments, a device access sheath 6 with an embedded electrode(s) connects to an extracorporeal energy source. In some embodiments, the energy source utilized includes RF, conductive heating, microwave, high frequency ultrasound, high intensity light (laser), or any combinations thereof. In some embodiments, activation or energization of the electrode induces coagulation at the disrupted tissue surface leading to sealing of disrupted surfaces. In some embodiments, the device access sheath 6 is manually retracted while energy is being applied to the embedded electrode(s) to induce coagulation along the access tract during retraction. In yet another embodiment, the device access sheath 6 is automatically retracted while energy is being applied to the embedded electrode(s). FIG. 4B shows the direction of retraction of the device access sheath 6, as indicated by the arrow.

### Ablation Delivery System

In some embodiments, the catheter device 4 comprises an ablation delivery system 22, as shown in FIGS. 2A-2B. In some embodiments, the ablation delivery system 22 provides an ablative energy or an ablative agent capable of killing cells in a mucosal layer of the gallbladder, killing the cells lining the cystic duct, or any combination thereof. In some embodiments, the ablative agent comprises a chemical agent, where the chemical agent is capable of killing cells in a mucosal layer of the gallbladder, killing the cells lining the cystic duct, or any combination thereof. Non-limiting examples of the chemical agent include an antibiotic, a liquid sclerosant, sodium tetradecyl sulphate, acetic acid, ethanol, hypertonic sodium chloride, and urea. In some embodiments, the ablation delivery system 22 comprises a low temperature thermal agent for cryoablation. In some embodiments, the ablation delivery system 22 comprises a cryoprobe through which a cooled, thermally conductive, fluid is circulated. In some embodiments, the ablation delivery system 22 comprises a high temperature thermal agent for thermal ablation, wherein the high temperature thermal agent is capable of killing cells in a mucosal layer of the gallbladder, killing the cells lining the cystic duct, or any combination thereof. In some embodiments, the device comprises a reservoir for storing the ablative agent. In some embodiments, the device comprises multiple ablation delivery systems located on different sections of the device. In some embodiments, the device comprises multiple ablation delivery systems of different ablation techniques.

In some embodiments, the ablation is spatially diffuse as compared to targeted ablation, such as cardiac ablation. In some embodiments, the spatially diffuse ablation allows for the whole internal lumen of the organ or most of the internal lumen of the organ to be ablated. In some embodiments, the ablating source for ablation of the cystic duct and inner mucosa includes, but is not limited to cryoablation, thermal ablation, and chemical ablation for defunctionalization of the gallbladder mucosa, for ablation or sclerosis of the cystic duct, or any combination thereof. In some embodiments, the ablation is thermal ablation, cryoablation, chemical ablation, or any combination thereof. In some embodiments, cryoablation comprises delivering a very low temperature fluid to wall of the gallbladder, such as liquid nitrogen. In some embodiments, cryoablation comprises delivering an ablation medium to the gallbladder wall that induces very low temperatures due to phase change, such as nitrous oxide or carbon dioxide. In some embodiments, thermal ablation comprises delivering a high temperature fluid to the wall of the gallbladder, such as steam. In some embodiments, the cryoablation and thermal ablation uses a spray application to deliver the fluid to the wall of the gallbladder. In some embodiments, chemical ablation comprises delivering one or more chemical agents that result in death of cells of the gallbladder wall. In some embodiments, the chemical agents are delivered in a liquid form, a fluid form, an aerosol form, a gel form, or any combination thereof.

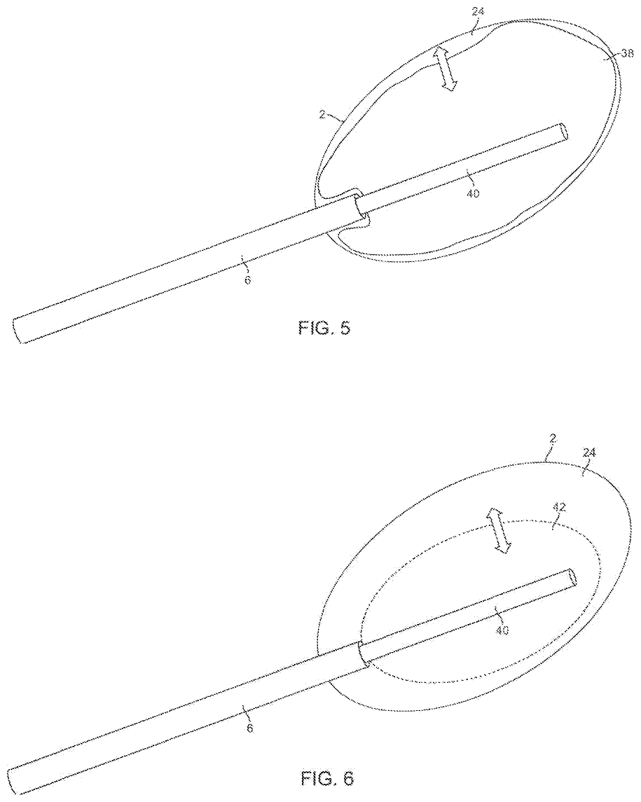

In some embodiments, the ablation delivery system comprises an ablation balloon 38, as seen in FIG. 5. In some embodiments, the ablation balloon 38 is a non-compliant balloon. In some embodiments, the ablation balloon 38 is a semi-compliant balloon. In some embodiments, the ablation balloon 38 is a compliant balloon. In some embodiments, the ablation balloon 38 is housed on an ablation balloon catheter 40 and deployed through an opening on the distal end 84 of the device access sheath 6, as shown in FIG. 5. In some embodiments, the ablation balloon 40 is in an unfurled configuration upon the catheter reaching the gallbladder 2 and is inflated within the gallbladder lumen 24. In some embodiments, the ablation balloon catheter 40 has a port (not shown in the figures) on the extracorporeal end that facilitates filling the ablation balloon with air or fluid (including, but not limited to water, saline, or contrast agent). In some embodiments, the ablation balloon 40 passively inflates with the introduction of an ablation medium. In some embodiments, in the inflated configuration, the ablation balloon 40 fills the gallbladder lumen 24. In some embodiments, the ablation balloon 40 does not exert pressure on the wall of the gallbladder 2 in the inflated configuration.

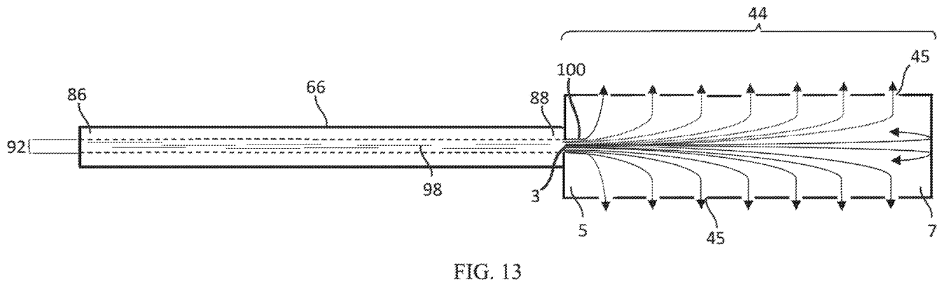

In some embodiments, the ablation balloon contains a cryogenic ablation medium and conductively ablates the gallbladder wall. In some embodiments, the balloon ablation catheter contains a delivery lumen for a liquid cryogenic ablation medium and an evacuation lumen to allow for removal of a gas cryogenic ablation medium and continuous introduction of energy. In some embodiments, the catheter lumen, in which the ablation medium is located, is sufficiently small as to minimize the hoop stress of the lumen due ablation supply pressure. FIG. 13 illustrates a catheter 66 comprising a catheter lumen 92 in which a cryogenic liquid ablation medium 98 is located and flows therethrough. In some embodiments, the catheter 66 comprises a catheter lumen 92 that is sufficiently small as to induce the cryogenic liquid ablation medium 98 to change into a cryogenic gas ablation medium 100 (i.e., a liquid-to-gas phase transition) at a phase change interface 3, as shown in FIG. 13. In some embodiments, the fenestrated nozzle 44 comprises a proximal end 5 and a distal end 7. In some embodiments, the phase change interface 3 is the area of the catheter lumen 92 located at the boundary between the catheter 66 and the fenestrated nozzle 44 where the liquid-to-gas phase transition of the cryogenic liquid ablation medium 98 occurs. In other words, in some embodiments, the phase change interface 3 is located at the distal end 88 of the catheter and at the proximal end 5 of the fenestrated nozzle 44. In some embodiments, the phase change interface 3 of the catheter is an area of the catheter where the lumen of the catheter decreases in diameter size. In some embodiments, after the cryogenic liquid ablation medium 98 has undergone the liquid-to-gas phase transition at the phase change interface 3, the cryogenic gas ablation medium 100 exits the fenestrated nozzle 44 via the plurality of fenestrations 45. In some embodiments, the cryogenic gas ablation medium 100 exits the fenestrated nozzle 44 via the plurality of fenestrations 45 and ablates the outer surface of the gallbladder lumen once the cryogenic gas ablation medium 100 upon contact with the tissue.

In some embodiments, the catheter lumen 92 size ranges from about 0.001 inches to about 0.1 inches. In some embodiments, the size of the catheter lumen 92 ranges from about 0.001 inches to about 0.002 inches, about 0.001 inches to about 0.003 inches, about 0.001 inches to about 0.004 inches, about 0.001 inches to about 0.005 inches, about 0.001 inches to about 0.006 inches, about 0.001 inches to about 0.0625 inches, about 0.001 inches to about 0.007 inches, about 0.001 inches to about 0.008 inches, about 0.001 inches to about 0.009 inches, about 0.001 inches to about 0.1 inches, about 0.002 inches to about 0.003 inches, about 0.002 inches to about 0.004 inches, about 0.002 inches to about 0.005 inches, about 0.002 inches to about 0.006 inches, about 0.002 inches to about 0.0625 inches, about 0.002 inches to about 0.007 inches, about 0.002 inches to about 0.008 inches, about 0.002 inches to about 0.009 inches, about 0.002 inches to about 0.1 inches, about 0.003 inches to about 0.004 inches, about 0.003 inches to about 0.005 inches, about 0.003 inches to about 0.006 inches, about 0.003 inches to about 0.0625 inches, about 0.003 inches to about 0.007 inches, about 0.003 inches to about 0.008 inches, about 0.003 inches to about 0.009 inches, about 0.003 inches to about 0.1 inches, about 0.004 inches to about 0.005 inches, about 0.004 inches to about 0.006 inches, about 0.004 inches to about 0.0625 inches, about 0.004 inches to about 0.007 inches, about 0.004 inches to about 0.008 inches, about 0.004 inches to about 0.009 inches, about 0.004 inches to about 0.1 inches, about 0.005 inches to about 0.006 inches, about 0.005 inches to about 0.0625 inches, about 0.005 inches to about 0.007 inches, about 0.005 inches to about 0.008 inches, about 0.005 inches to about 0.009 inches, about 0.005 inches to about 0.1 inches, about 0.006 inches to about 0.0625 inches, about 0.006 inches to about 0.007 inches, about 0.006 inches to about 0.008 inches, about 0.006 inches to about 0.009 inches, about 0.006 inches to about 0.1 inches, about 0.0625 inches to about 0.007 inches, about 0.0625 inches to about 0.008 inches, about 0.0625 inches to about 0.009 inches, about 0.0625 inches to about 0.1 inches, about 0.007 inches to about 0.008 inches, about 0.007 inches to about 0.009 inches, about 0.007 inches to about 0.1 inches, about 0.008 inches to about 0.009 inches, about 0.008 inches to about 0.1 inches, or about 0.009 inches to about 0.1 inches. In some embodiments, the size of the catheter lumen 92 ranges from about 0.001 inches, about 0.002 inches, about 0.003 inches, about 0.004 inches, about 0.005 inches, about 0.006 inches, about 0.0625 inches, about 0.007 inches, about 0.008 inches, about 0.009 inches, or about 0.1 inches. In some embodiments, the size of the catheter lumen 92 ranges from at least about 0.001 inches, about 0.002 inches, about 0.003 inches, about 0.004 inches, about 0.005 inches, about 0.006 inches, about 0.0625 inches, about 0.007 inches, about 0.008 inches, or about 0.009 inches. In some embodiments, the size of the catheter lumen 92 ranges from at most about 0.002 inches, about 0.003 inches, about 0.004 inches, about 0.005 inches, about 0.006 inches, about 0.0625 inches, about 0.007 inches, about 0.008 inches, about 0.009 inches, or about 0.1 inches.

In some embodiments, the ablation balloon 40 in the inflated configuration fills more than 50%, 60%, 70%, 80%, 90%, 95%, or 99% of the interior volume of the gallbladder 2. In some embodiments, the ablation balloon 40, in the inflated configuration, fills about 50% to about 99% of the interior volume of the gallbladder 2. In some embodiments, the ablation balloon 40, in the inflated configuration, fills about 50% to about 60%, about 50% to about 70%, about 50% to about 80%, about 50% to about 85%, about 50% to about 90%, about 50% to about 95%, about 50% to about 96%, about 50% to about 97%, about 50% to about 98%, about 50% to about 99%, about 60% to about 70%, about 60% to about 80%, about 60% to about 85%, about 60% to about 90%, about 60% to about 95%, about 60% to about 96%, about 60% to about 97%, about 60% to about 98%, about 60% to about 99%, about 70% to about 80%, about 70% to about 85%, about 70% to about 90%, about 70% to about 95%, about 70% to about 96%, about 70% to about 97%, about 70% to about 98%, about 70% to about 99%, about 80% to about 85%, about 80% to about 90%, about 80% to about 95%, about 80% to about 96%, about 80% to about 97%, about 80% to about 98%, about 80% to about 99%, about 85% to about 90%, about 85% to about 95%, about 85% to about 96%, about 85% to about 97%, about 85% to about 98%, about 85% to about 99%, about 90% to about 95%, about 90% to about 96%, about 90% to about 97%, about 90% to about 98%, about 90% to about 99%, about 95% to about 96%, about 95% to about 97%, about 95% to about 98%, about 95% to about 99%, about 96% to about 97%, about 96% to about 98%, about 96% to about 99%, about 97% to about 98%, about 97% to about 99%, or about 98% to about 99% of the interior volume of the gallbladder 2. In some embodiments, the ablation balloon 40, in the inflated configuration, fills about 50%, about 60%, about 70%, about 80%, about 85%, about 90%, about 95%, about 96%, about 97%, about 98%, or about 99% of the interior volume of the gallbladder 2. In some embodiments, the ablation balloon 40, in the inflated configuration, fills at least about 50%, about 60%, about 70%, about 80%, about 85%, about 90%, about 95%, about 96%, about 97%, or about 98% of the interior volume of the gallbladder 2. In some embodiments, the ablation balloon 40, in the inflated configuration, fills at most about 60%, about 70%, about 80%, about 85%, about 90%, about 95%, about 96%, about 97%, about 98%, or about 99% of the interior volume of the gallbladder 2.

### Ablation Media

In some embodiments, the ablation balloon 38 comprises an ablation medium. In some embodiments, the ablation medium is a fluid. In some embodiments, the ablation medium is a gas. In some embodiments, the ablation medium is a thermal ablation medium. Non-limiting examples of the thermal ablation medium include saline, water, air, glycerin, steam, and dextrose. In some embodiments, the temperature of the thermal ablation medium is controlled by the extracorporeal control unit 20.

In some embodiments, the temperature of the thermal ablation medium ranges from about 37 degrees Celsius to about 100 degrees Celsius when the thermal ablation medium is used with the catheter devices disclosed herein. In some embodiments, the temperature of the thermal ablation medium ranges from about 37 degrees Celsius to about 38 degrees Celsius, about 37 degrees Celsius to about 40 degrees Celsius, about 37 degrees Celsius to about 45 degrees Celsius, about 37 degrees Celsius to about 50 degrees Celsius, about 37 degrees Celsius to about 55 degrees Celsius, about 37 degrees Celsius to about 60 degrees Celsius, about 37 degrees Celsius to about 70 degrees Celsius, about 37 degrees Celsius to about 80 degrees Celsius, about 37 degrees Celsius to about 90 degrees Celsius, about 37 degrees Celsius to about 100 degrees Celsius, about 37 degrees Celsius to about 100 degrees Celsius, about 38 degrees Celsius to about 40 degrees Celsius, about 38 degrees Celsius to about 45 degrees Celsius, about 38 degrees Celsius to about 50 degrees Celsius, about 38 degrees Celsius to about 55 degrees Celsius, about 38 degrees Celsius to about 60 degrees Celsius, about 38 degrees Celsius to about 70 degrees Celsius, about 38 degrees Celsius to about 80 degrees Celsius, about 38 degrees Celsius to about 90 degrees Celsius, about 38 degrees Celsius to about 100 degrees Celsius, about 38 degrees Celsius to about 100 degrees Celsius, about 40 degrees Celsius to about 45 degrees Celsius, about 40 degrees Celsius to about 50 degrees Celsius, about 40 degrees Celsius to about 55 degrees Celsius, about 40 degrees Celsius to about 60 degrees Celsius, about 40 degrees Celsius to about 70 degrees Celsius, about 40 degrees Celsius to about 80 degrees Celsius, about 40 degrees Celsius to about 90 degrees Celsius, about 40 degrees Celsius to about 100 degrees Celsius, about 45 degrees Celsius to about 50 degrees Celsius, about 45 degrees Celsius to about 55 degrees Celsius, about 45 degrees Celsius to about 60 degrees Celsius, about 45 degrees Celsius to about 70 degrees Celsius, about 45 degrees Celsius to about 80 degrees Celsius, about 45 degrees Celsius to about 90 degrees Celsius, about 45 degrees Celsius to about 100 degrees Celsius, about 45 degrees Celsius to about 100 degrees Celsius, about 50 degrees Celsius to about 55 degrees Celsius, about 50 degrees Celsius to about 60 degrees Celsius, about 50 degrees Celsius to about 70 degrees Celsius, about 50 degrees Celsius to about 80 degrees Celsius, about 50 degrees Celsius to about 90 degrees Celsius, about 50 degrees Celsius to about 100 degrees Celsius, about 50 degrees Celsius to about 100 degrees Celsius, about 55 degrees Celsius to about 60 degrees Celsius, about 55 degrees Celsius to about 70 degrees Celsius, about 55 degrees Celsius to about 80 degrees Celsius, about 55 degrees Celsius to about 90 degrees Celsius, about 55 degrees Celsius to about 100 degrees Celsius, about 55 degrees Celsius to about 100 degrees Celsius, about 60 degrees Celsius to about 70 degrees Celsius, about 60 degrees Celsius to about 80 degrees Celsius, about 60 degrees Celsius to about 90 degrees Celsius, about 60 degrees Celsius to about 100 degrees Celsius, about 60 degrees Celsius to about 100 degrees Celsius, about 70 degrees Celsius to about 80 degrees Celsius, about 70 degrees Celsius to about 90 degrees Celsius, about 70 degrees Celsius to about 100 degrees Celsius, about 80 degrees Celsius to about 90 degrees Celsius, about 80 degrees Celsius to about 100 degrees Celsius, about 90 degrees Celsius to about 100 degrees Celsius when the thermal ablation medium is used with the catheter devices disclosed herein. In some embodiments, the temperature of the thermal ablation medium ranges from about 37 degrees Celsius, about 38 degrees Celsius, about 40 degrees Celsius, about 45 degrees Celsius, about 50 degrees Celsius, about 55 degrees Celsius, about 60 degrees Celsius, about 70 degrees Celsius, about 80 degrees Celsius, about 90 degrees Celsius, or about 100 degrees Celsius when the thermal ablation medium is used with the catheter devices disclosed herein. In some embodiments, the temperature of the thermal ablation medium ranges from at least about 37 degrees Celsius, about 38 degrees Celsius, about 40 degrees Celsius, about 45 degrees Celsius, about 50 degrees Celsius, about 55 degrees Celsius, about 60 degrees Celsius, about 70 degrees Celsius, about 80 degrees Celsius, about 90 degrees Celsius, or about 100 degrees Celsius when the thermal ablation medium is used with the catheter devices disclosed herein. In some embodiments, the temperature of the thermal ablation medium ranges from at most about 38 degrees Celsius, about 40 degrees Celsius, about 45 degrees Celsius, about 50 degrees Celsius, about 55 degrees Celsius, about 60 degrees Celsius, about 70 degrees Celsius, about 80 degrees Celsius, about 90 degrees Celsius, or about 100 degrees Celsius when the thermal ablation medium is used with the catheter devices disclosed herein.

In some embodiments, the ablation medium is a cryogenic ablation medium. In some embodiments, the cryogenic ablation medium is a liquid. In some embodiments, the cryogenic ablation medium is a gas. In some embodiments, the cryogenic ablation medium undergoes a liquid-to-gas phase transition when being delivered using the catheter devices disclosed herein. In some embodiments, cryoablation is achieved via the refrigerant property due to the liquid to gas phase change from an ablation medium, such as liquid nitrous oxide, carbon dioxide, and argon. In some embodiments, the phase change of the cryogenic ablation medium is triggered by

In some embodiments, the cryogenic ablation medium is nitrous oxide. Non-limiting examples of the cryogenic ablation medium include nitrous oxide, nitrogen, carbon dioxide, and argon. In some embodiments, the temperature of the cryogenic ablation medium is controlled by the extracorporeal control unit 20. In some embodiments, the pressure of the cryogenic ablation medium is controlled by the extracorporeal control unit 20. In some embodiments, the final volume of the cryogenic ablation medium increases up to about 600 times the original volume of the cryogenic medium. In some embodiments, the final volume of the cryogenic ablation medium is the volume of the cryogenic ablation medium once it is delivered by the catheter device (e.g., once it is sprayed onto the surface of the gallbladder lumen). In some embodiments, the initial volume of the cryogenic ablation medium is the volume of the cryogenic ablation medium before it is delivered by the catheter device (e.g., when it is contained in a vessel outside of the body). In some embodiments, the final state of the cryogenic ablation medium is a gas phase. In some embodiments, the initial state of the cryogenic ablation medium is a liquid phase. In some embodiments, the extracorporeal control unit 20 monitors and controls the pressure of the cryogenic ablation medium in a gas phase, in real time. In some embodiments, the extracorporeal control unit 20 monitors and controls the pressure of the cryogenic ablation medium via a pressure sensor.

In some embodiments, the temperature of the cryogenic ablation medium ranges from about −120 degrees Celsius to about 0 degrees Celsius when the cryogenic ablation medium is used with the catheter devices disclosed herein. In some embodiments, the temperature of the cryogenic ablation medium ranges from about −120 degrees Celsius to about −110 degrees Celsius, about −120 degrees Celsius to about −100 degrees Celsius, about −120 degrees Celsius to about −90 degrees Celsius, about −120 degrees Celsius to about −80 degrees Celsius, about −120 degrees Celsius to about −70 degrees Celsius, about −120 degrees Celsius to about −60 degrees Celsius, about −120 degrees Celsius to about −50 degrees Celsius, about −120 degrees Celsius to about −40 degrees Celsius, about −120 degrees Celsius to about −30 degrees Celsius, about −120 degrees Celsius to about −20 degrees Celsius, about −120 degrees Celsius to about 0 degrees Celsius, about −110 degrees Celsius to about −100 degrees Celsius, about −110 degrees Celsius to about −90 degrees Celsius, about −110 degrees Celsius to about −80 degrees Celsius, about −110 degrees Celsius to about −70 degrees Celsius, about −110 degrees Celsius to about −60 degrees Celsius, about −110 degrees Celsius to about −50 degrees Celsius, about −110 degrees Celsius to about −40 degrees Celsius, about −110 degrees Celsius to about −30 degrees Celsius, about −110 degrees Celsius to about −20 degrees Celsius, about −110 degrees Celsius to about 0 degrees Celsius, about −100 degrees Celsius to about −90 degrees Celsius, about −100 degrees Celsius to about −80 degrees Celsius, about −100 degrees Celsius to about −70 degrees Celsius, about −100 degrees Celsius to about −60 degrees Celsius, about −100 degrees Celsius to about −50 degrees Celsius, about −100 degrees Celsius to about −40 degrees Celsius, about −100 degrees Celsius to about −30 degrees Celsius, about −100 degrees Celsius to about −20 degrees Celsius, about −100 degrees Celsius to about 0 degrees Celsius, about −90 degrees Celsius to about −80 degrees Celsius, about −90 degrees Celsius to about −70 degrees Celsius, about −90 degrees Celsius to about −60 degrees Celsius, about −90 degrees Celsius to about −50 degrees Celsius, about −90 degrees Celsius to about −40 degrees Celsius, about −90 degrees Celsius to about −30 degrees Celsius, about −90 degrees Celsius to about −20 degrees Celsius, about −90 degrees Celsius to about 0 degrees Celsius, about −80 degrees Celsius to about −70 degrees Celsius, about −80 degrees Celsius to about −60 degrees Celsius, about −80 degrees Celsius to about −50 degrees Celsius, about −80 degrees Celsius to about −40 degrees Celsius, about −80 degrees Celsius to about −30 degrees Celsius, about −80 degrees Celsius to about −20 degrees Celsius, about −80 degrees Celsius to about 0 degrees Celsius, about −70 degrees Celsius to about −60 degrees Celsius, about −70 degrees Celsius to about −50 degrees Celsius, about −70 degrees Celsius to about −40 degrees Celsius, about −70 degrees Celsius to about −30 degrees Celsius, about −70 degrees Celsius to about −20 degrees Celsius, about −70 degrees Celsius to about 0 degrees Celsius, about −60 degrees Celsius to about −50 degrees Celsius, about −60 degrees Celsius to about −40 degrees Celsius, about −60 degrees Celsius to about −30 degrees Celsius, about −60 degrees Celsius to about −20 degrees Celsius, about −60 degrees Celsius to about 0 degrees Celsius, about −50 degrees Celsius to about −40 degrees Celsius, about −50 degrees Celsius to about −30 degrees Celsius, about −50 degrees Celsius to about −20 degrees Celsius, about −50 degrees Celsius to about 0 degrees Celsius, about −40 degrees Celsius to about −30 degrees Celsius, about −40 degrees Celsius to about −20 degrees Celsius, about −40 degrees Celsius to about 0 degrees Celsius, about −30 degrees Celsius to about −20 degrees Celsius, about −30 degrees Celsius to about 0 degrees Celsius, or about −20 degrees Celsius to about 0 degrees Celsius when the cryogenic ablation medium is used with the catheter devices disclosed herein. In some embodiments, the temperature of the cryogenic ablation medium ranges from about −120 degrees Celsius, about −110 degrees Celsius, about −100 degrees Celsius, about −90 degrees Celsius, about −80 degrees Celsius, about −70 degrees Celsius, about −60 degrees Celsius, about-50 degrees Celsius, about −40 degrees Celsius, about −30 degrees Celsius, about −20 degrees Celsius, or about 0 degrees Celsius when the cryogenic ablation medium is used with the catheter devices disclosed herein. In some embodiments, the temperature of the cryogenic ablation medium ranges from at least about −120 degrees Celsius, about −110 degrees Celsius, about −100 degrees Celsius, about −90 degrees Celsius, about −80 degrees Celsius, about −70 degrees Celsius, about −60 degrees Celsius, about −50 degrees Celsius, about −40 degrees Celsius, about −30 degrees Celsius, or about −20 degrees Celsius when the cryogenic ablation medium is used with the catheter devices disclosed herein. In some embodiments, the temperature of the cryogenic ablation medium ranges from at most about −110 degrees Celsius, about −100 degrees Celsius, about −90 degrees Celsius, about −80 degrees Celsius, about −70 degrees Celsius, about −60 degrees Celsius, about −50 degrees Celsius, about −40 degrees Celsius, about −30 degrees Celsius, about −20 degrees Celsius, or about 0 degrees Celsius when the cryogenic ablation medium is used with the catheter devices disclosed herein.

In some embodiments, the ablation balloon 38 is a cryogenic ablation balloon. In some embodiments, the cryogenic ablation balloon comprises a cryogenic ablation medium. In some embodiments, the ablation delivery system 22 comprises a highly compliant cryoablation balloon introduced into the gallbladder lumen via a catheter and ablates the mucosal tissue layer. In some embodiments, the ablation balloon 38 achieves apposition through a highly compliant, low durometer construction, which allows for variability in the diameter gallbladder lumen across patients. In some embodiments, multi-lumen tubing acts to introduce the cryogen medium into the ablation balloon 38 through one lumen, while evacuating through the other. In some embodiments, the ablation balloon 38 contains at least one pressure sensor to create a closed-loop feedback system in which a maximum balloon pressure is maintained. In some embodiments, the ablation balloon 38 contains at least one temperature sensor, located either on the outer balloon surface or centrally, to monitor ablation temperatures.

In some embodiments, the ablation balloon 38 is a thermal ablation balloon. In some embodiments, the thermal ablation balloon comprises a thermal ablation medium. In some embodiments, the ablation delivery system 22 comprises a highly compliant thermal ablation balloon introduced into the gallbladder lumen via a catheter and ablates the mucosal tissue layer. In some embodiments, the balloon achieves apposition through a highly compliant, low durometer construction, which allows for variability in the diameter gallbladder lumen across patients. In some embodiments, the thermal energy source is a hot medium located inside the balloon and is generated by cycling the medium through an external heating source, conductive heating, or electromagnetic heating within the balloon. In some embodiments, in the case of a circulating heating source, a multi-lumen tubing acts to introduce fluid through one lumen, while evacuating fluid through another. In some embodiments, in the case of conductive heating, an internal mechanical mixer is located on a central catheter lumen to promote uniform medium heating. In some embodiments, in the case of electromagnetic heating, a unipolar or bipolar energy source is used to generate an electromagnetic field in the presence of a medium with ionic properties. In some embodiments, the field generates thermal heat from friction of the mechanical ion movement. In some embodiments, the heating medium is located between two balloon layers in order to reduce the energy required to reach thermal ablation temperatures. In some embodiments, the medium is introduced to the balloon to create a distinct ablation shape or pattern, based upon the anatomy of the patient or target area. In some embodiments, the heating element is coupled to a thermal switch, which turns off energy output when a set temperature or temperature range has been reached. In some embodiments, a thermocouple/thermistor relays temperature back to the energy source and modulates ablation power based upon a closed loop feedback system. In some embodiments, the balloon is a spiral shape and contour to the lumen of the gallbladder while maximizing apposition. In some embodiments, the thermal medium is a material with low specific heat and a high flash point, such as glycerin, in order to quickly transmit energy to the ablation zone and reduce thermal damage due to lag.

In some embodiments, the ablation delivery system comprises a balloon that has various material properties. In some embodiments, the ablation balloon is a compliant ablation balloon. The compliant ablation balloon comprises a soft, flexible material and conforms to the shape of the gallbladder when inflated. In some embodiments, the ablation balloon is a semi-compliant ablation balloon. The semi-compliant ablation balloon comprises a semi-flexible material that generally conforms to the shape of the gallbladder when inflated. In some embodiments, the ablation balloon is a non-compliant ablation balloon. The non-compliant ablation balloon comprises a less flexible material that does not conform to the shape of an outer container. In the inflated configuration, the non-compliant ablation balloon maintains its shape and resists deformation. In some embodiments, the ablation balloon has a thickness of at least 1 micrometer (μm), 10 μm, 100 μm, 1 millimeter (mm), or 10 mm.

In some embodiments, the ablation balloon is configured to deliver the ablative energy or ablative agent to the mucosal layer of the gallbladder. In some embodiments, the ablation balloon is porous, where the ablative energy or ablative agent is delivered to the mucosal layer through the fenestrated ablation balloon 42, as seen in FIG. 6. In some embodiments, the fenestrated ablation balloon 42 comprises a plurality of fenestrations. In some embodiments, the plurality of fenestrations of the fenestrated ablation balloon 42 allow for an ablation medium to exit the fenestrated ablation balloon 42 and enter the gallbladder lumen 24. In some embodiments, the fenestrated ablation balloon 42 has a volume that is smaller than the volume of the gallbladder, as shown in FIG. 6. In some embodiments, the outer surface of the fenestrated ablation balloon 42 does not come in contact with the outer surface of the gallbladder lumen 24, as shown in FIG. 6. In yet another embodiment, the fenestrated ablation balloon 42 has a volume that about the same than the volume of the gallbladder. In some embodiments, the outer surface of the fenestrated ablation balloon 42 comes in direct contact with the outer surface of the gallbladder lumen 24. In some embodiments, the fenestrated ablation balloon 42 is inflated with an ablation medium. In some embodiments, the ablation medium comes in contact with the outer surface of the gallbladder lumen 24 by exiting the fenestrated ablation balloon 42 through the plurality of fenestrations on the surface of the fenestrated ablation balloon 42.

In some embodiments, the fenestrated ablation balloon is configured to convectively ablate a surrounding tissue. In some embodiments, the fenestrated ablation balloon convectively ablates a surrounding tissue by delivering an ablation medium into a lumen of a tissue (e.g., into a gallbladder lumen). In some embodiments, the fenestrated ablation balloon delivers an ablation medium into a lumen of a tissue (e.g., into a gallbladder lumen) via the plurality of fenestrations of the fenestrated ablation balloon. In some embodiments, a catheter is used to transport or deliver the ablation medium from an ablation medium reservoir (e.g., an extracorporeal ablation medium reservoir) to the lumen of the fenestrated ablation balloon. In some embodiments, the catheter transporting or delivering the ablation medium into the lumen of the fenestrated ablation balloon is a fenestrated catheter. In some embodiments, the catheter transporting or delivering the ablation medium into the lumen of the fenestrated ablation balloon is a catheter comprising a fenestrated nozzle. In some embodiments, the catheter transporting or delivering the ablation medium into the lumen of the fenestrated ablation balloon is not a fenestrated catheter. In some embodiments, the catheter transporting or delivering the ablation medium into the lumen of the fenestrated ablation balloon is a catheter comprising a distal opening. In some embodiments, the catheter transporting or delivering the ablation medium into the lumen of the fenestrated ablation balloon is a catheter comprising a sprayer, a spray applicator, an irrigator, or any combination thereof.

In some embodiments, the ablative energy or ablative agent is delivered to the mucosal layer of the gallbladder 2 by transfer of the ablative energy or ablative agent from the ablation source to the surface of the ablation balloon. In some embodiments, the ablative energy or ablative agent is delivered to the mucosal layer of the gallbladder 2 through one or more delivery lumens along the elongated body of the catheter, where the delivery lumens are positioned within the gallbladder. In some embodiments, the ablation catheter sprays the cryogenic ablation medium into a porous balloon, which helps deliver the cryogenic ablation medium uniformly onto the gallbladder wall. In some embodiments, the ablation medium is sprayed within the porous balloon via fenestrations within the ablation catheter body, located within the balloon.

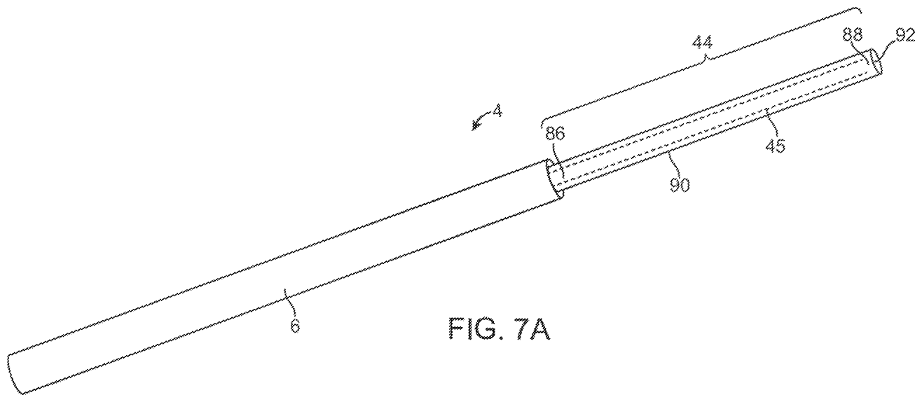

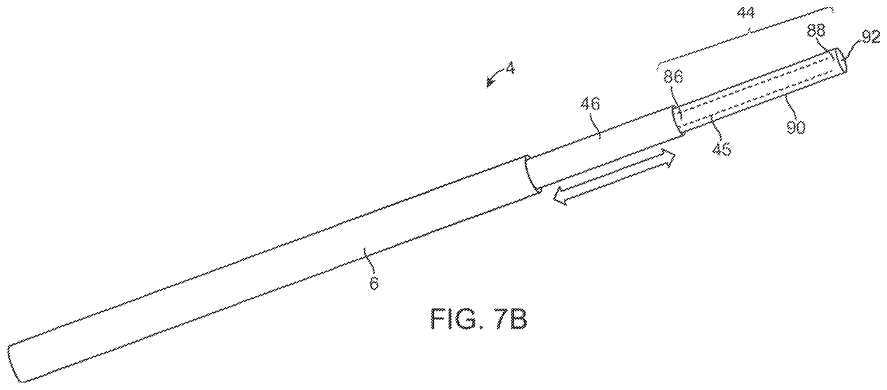

In some embodiments, the ablation delivery system is a catheter 66 with fenestrations 45, as seen in FIG. 7A. In some embodiments, the catheter 66 is an elongated, flexible tube having an outer surface 90, a proximal end 86, a distal end 88, an inner surface (not shown in the figures), and a lumen 92 that is bound by the inner surface between the proximal end 86 and the distal end 88. In some embodiments, the catheter 66 comprises a fenestrated catheter nozzle 44. In some embodiments, the fenestrated catheter nozzle 44 comprises a plurality of fenestrations 45. In some embodiments, the fenestrated catheter nozzle 44 is a fenestrated area of the catheter 66 located near the distal end 88 of the catheter 66. In some embodiments, the fenestrations 45 are configured to direct a flow path of an ablation medium (e.g., a fluid, a gas, or any combination thereof) expelled by the fenestrated catheter nozzle 44, across the outer surface 90 of the catheter 66. In some embodiments, the fenestrations 45 are configured to direct a flow path of an ablation medium (e.g., a fluid, a gas, or any combination thereof) expelled by the catheter 66, across the outer surface 90 of the catheter 66. In some embodiments, the ablation medium (e.g., a fluid, a gas, or any combination thereof) expelled by the fenestrated catheter nozzle 44, by the catheter 66, or any combination thereof is a thermal medium. In some embodiments, the ablation medium (e.g., a fluid, a gas, or any combination thereof) expelled by the fenestrated catheter nozzle 44, by the catheter 66, or any combination thereof is a cryogen. In some embodiments, the ablation catheter delivers a liquid ablation medium to the hollow, fenestrated end of the catheter (i.e., the fenestrated nozzle 44), whereby the pressure from the phase change drives the flow of the aerosolized ablation medium radially outwards through the fenestrations. In some instances, the catheter allows for a heated ablation medium to be sprayed into the gallbladder cavity. In some instances, the catheter allows for a cold ablation medium to be sprayed into the gallbladder cavity.

In some embodiments, the catheter comprises fenestrations 45 located at the distal end 88 of the catheter. In some embodiments, the catheter 66 comprises fenestrations 45 at the proximal end 86 of the catheter. In some embodiments, the fenestrations 45 are located throughout the elongated body of the catheter 66. In some instances, the fenestrations 45 span the full circumference of the catheter 66. In some instances, the fenestrations 45 span about 10% to about 100% of the circumference of the catheter 66. In some instances, the fenestrations 45 span about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100% of the circumference of the catheter 66. In some instances, the fenestrations 45 span about 10%, about 20%, about 30%, about 40%, about 50%, about 60%, about 70%, about 80%, about 90%, or about 100% of the circumference of the catheter 66. In some instances, the fenestrations 45 span at least about 10%, about 20%, about 30%, about 40%, about 50%, about 60%, about 70%, about 80%, or about 90% of the circumference of the catheter 66. In some instances, the fenestrations 45 span at most about 20%, about 30%, about 40%, about 50%, about 60%, about 70%, about 80%, about 90%, or about 100% of the circumference of the catheter 66.

In some embodiments, the catheter nozzle 44 occupies a fraction of the total surface area of the catheter 66. In some embodiments, the fenestrations 45 occupy about 10% to about 100% of the total surface area of the catheter 66. In some embodiments, the fenestrations 45 occupy about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100% of the total surface area of the catheter 66. In some embodiments, the fenestrations 45 occupy about 10%, about 20%, about 30%, about 40%, about 50%, about 60%, about 70%, about 80%, about 90%, or about 100% of the total surface area of the catheter 66. In some embodiments, the fenestrations 45 occupy at least about 10%, about 20%, about 30%, about 40%, about 50%, about 60%, about 70%, about 80%, or about 90% of the total surface area of the catheter 66. In some embodiments, the fenestrations 45 occupy at most about 20%, about 30%, about 40%, about 50%, about 60%, about 70%, about 80%, about 90%, or about 100% of the total surface area of the catheter 66.

In some instances, the fenestrations span the full circumference of the catheter between about 0 cm and about 10 cm of the distal end. In some instances, the fenestrations span the full circumference of the catheter between about 1 cm and about 10 cm of the distal end. In some instances, the fenestrations span the full circumference of the catheter between about 2 cm and about 10 cm of the distal end. In some instances, the fenestrations span the full circumference of the catheter between about 3 cm and about 10 cm of the distal end. In some instances, the fenestrations span the full circumference of the catheter between about 4 cm and about 10 cm of the distal end. In some instances, the fenestrations span the full circumference of the catheter between about 5 cm and about 10 cm of the distal end. In some instances, the fenestrations span the full circumference of the catheter between about 6 cm and about 10 cm of the distal end. In some instances, the fenestrations span the full circumference of the catheter between about 7 cm and about 10 cm of the distal end. In some instances, the fenestrations span the full circumference of the catheter between about 8 cm and about 10 cm of the distal end. In some instances, the fenestrations span the full circumference of the catheter between about 9 cm and about 10 cm of the distal end.

In some embodiments, the length of the fenestrated catheter nozzle 44 ranges from about 1 cm to about 10 cm. In some embodiments, the length of the fenestrated catheter nozzle 44 ranges from about 1 cm to about 20 cm. In some embodiments, the length of the fenestrated catheter nozzle 44 ranges from about 1 cm to about 2 cm, about 1 cm to about 3 cm, about 1 cm to about 4 cm, about 1 cm to about 5 cm, about 1 cm to about 6 cm, about 1 cm to about 7 cm, about 1 cm to about 8 cm, about 1 cm to about 9 cm, about 1 cm to about 10 cm, about 1 cm to about 15 cm, about 1 cm to about 20 cm, about 2 cm to about 3 cm, about 2 cm to about 4 cm, about 2 cm to about 5 cm, about 2 cm to about 6 cm, about 2 cm to about 7 cm, about 2 cm to about 8 cm, about 2 cm to about 9 cm, about 2 cm to about 10 cm, about 2 cm to about 15 cm, about 2 cm to about 20 cm, about 3 cm to about 4 cm, about 3 cm to about 5 cm, about 3 cm to about 6 cm, about 3 cm to about 7 cm, about 3 cm to about 8 cm, about 3 cm to about 9 cm, about 3 cm to about 10 cm, about 3 cm to about 15 cm, about 3 cm to about 20 cm, about 4 cm to about cm, about 4 cm to about 6 cm, about 4 cm to about 7 cm, about 4 cm to about 8 cm, about 4 cm to about 9 cm, about 4 cm to about 10 cm, about 4 cm to about 15 cm, about 4 cm to about 20 cm, about 5 cm to about 6 cm, about 5 cm to about 7 cm, about 5 cm to about 8 cm, about 5 cm to about 9 cm, about 5 cm to about 10 cm, about 5 cm to about 15 cm, about 5 cm to about 20 cm, about 6 cm to about 7 cm, about 6 cm to about 8 cm, about 6 cm to about 9 cm, about 6 cm to about 10 cm, about 6 cm to about 15 cm, about 6 cm to about 20 cm, about 7 cm to about 8 cm, about 7 cm to about 9 cm, about 7 cm to about 10 cm, about 7 cm to about 15 cm, about 7 cm to about 20 cm, about 8 cm to about 9 cm, about 8 cm to about 10 cm, about 8 cm to about 15 cm, about 8 cm to about 20 cm, about 9 cm to about 10 cm, about 9 cm to about 15 cm, about 9 cm to about 20 cm, about 10 cm to about 15 cm, about 10 cm to about 20 cm, or about 15 cm to about 20 cm. In some embodiments, the length of the fenestrated catheter nozzle 44 ranges from about 1 cm, about 2 cm, about 3 cm, about 4 cm, about 5 cm, about 6 cm, about 7 cm, about 8 cm, about 9 cm, about 10 cm, about 15 cm, or about 20 cm. In some embodiments, the length of the fenestrated catheter nozzle 44 ranges from at least about 1 cm, about 2 cm, about 3 cm, about 4 cm, about 5 cm, about 6 cm, about 7 cm, about 8 cm, about 9 cm, about 10 cm, or about 15 cm. In some embodiments, the length of the fenestrated catheter nozzle 44 ranges from at most about 2 cm, about 3 cm, about 4 cm, about 5 cm, about 6 cm, about 7 cm, about 8 cm, about 9 cm, about cm, about 15 cm, or about 20 cm.

In some embodiments, the length of the catheter 66 ranges from about 10 cm to about 80 cm. In some embodiments, the length of the catheter 66 ranges from about 10 cm to about 15 cm, about 10 cm to about 20 cm, about 10 cm to about 25 cm, about 10 cm to about 30 cm, about 10 cm to about 35 cm, about 10 cm to about 40 cm, about 10 cm to about 45 cm, about 10 cm to about 50 cm, about 10 cm to about 55 cm, about 10 cm to about 60 cm, about 10 cm to about 80 cm, about 15 cm to about 20 cm, about 15 cm to about 25 cm, about 15 cm to about 30 cm, about cm to about 35 cm, about 15 cm to about 40 cm, about 15 cm to about 45 cm, about 15 cm to about 50 cm, about 15 cm to about 55 cm, about 15 cm to about 60 cm, about 15 cm to about 80 cm, about 20 cm to about 25 cm, about 20 cm to about 30 cm, about 20 cm to about 35 cm, about cm to about 40 cm, about 20 cm to about 45 cm, about 20 cm to about 50 cm, about 20 cm to about 55 cm, about 20 cm to about 60 cm, about 20 cm to about 80 cm, about 25 cm to about 30 cm, about 25 cm to about 35 cm, about 25 cm to about 40 cm, about 25 cm to about 45 cm, about cm to about 50 cm, about 25 cm to about 55 cm, about 25 cm to about 60 cm, about 25 cm to about 80 cm, about 30 cm to about 35 cm, about 30 cm to about 40 cm, about 30 cm to about 45 cm, about 30 cm to about 50 cm, about 30 cm to about 55 cm, about 30 cm to about 60 cm, about 30 cm to about 80 cm, about 35 cm to about 40 cm, about 35 cm to about 45 cm, about 35 cm to about 50 cm, about 35 cm to about 55 cm, about 35 cm to about 60 cm, about 35 cm to about 80 cm, about 40 cm to about 45 cm, about 40 cm to about 50 cm, about 40 cm to about 55 cm, about 40 cm to about 60 cm, about 40 cm to about 80 cm, about 45 cm to about 50 cm, about 45 cm to about 55 cm, about 45 cm to about 60 cm, about 45 cm to about 80 cm, about 50 cm to about 55 cm, about 50 cm to about 60 cm, about 50 cm to about 80 cm, about 55 cm to about 60 cm, about 55 cm to about 80 cm, or about 60 cm to about 80 cm. In some embodiments, the length of the catheter 66 ranges from about 10 cm, about 15 cm, about 20 cm, about 25 cm, about 30 cm, about 35 cm, about 40 cm, about 45 cm, about 50 cm, about 55 cm, about 60 cm, or about 80 cm. In some embodiments, the length of the catheter 66 ranges from at least about 10 cm, about 15 cm, about 20 cm, about 25 cm, about 30 cm, about 35 cm, about 40 cm, about 45 cm, about 50 cm, about 55 cm, or about 60 cm. In some embodiments, the length of the catheter 66 ranges from at most about 15 cm, about 20 cm, about 25 cm, about 30 cm, about 35 cm, about 40 cm, about 45 cm, about 50 cm, about 55 cm, about 60 cm, or about 80 cm.

In some embodiments, the fenestrations 45 extend along the outside surface of the catheter 66. In some embodiments, the fenestrations 45 are arranged in a pattern along the outside surface of the catheter 66. In some embodiments, the pattern is a linear pattern, a hexagonal pattern, a rectangular pattern, a triangular pattern, a square pattern, a circular pattern, a spiral pattern, or any combination thereof. In some embodiments, the In some embodiments, the size, shape, or any combination thereof of the fenestrations 45 are varied in order to optimize flow of an ablation medium (e.g., a fluid, a gas, or any combination thereof). In some embodiments, the shape of the fenestrations 45 is circular. In some embodiments, the shape of the fenestrations 45 is non-circular. In some embodiments, the shape of the fenestrations 45 is circular, elliptical, triangular, rectangular, square, or any combination thereof. In some embodiments, the fenestrations 45 are micro-drilled or laser-drilled in the catheter wall 90 or are formed by any other conventional method known by the skilled artisan.

In some embodiments, the diameter of each of the fenestrations 45 ranges from about 0.001 cm to about 0.5 cm. In some embodiments, the diameter of each of the fenestrations 45 ranges from about 0.001 cm to about 0.005 cm, about 0.001 cm to about 0.01 cm, about 0.001 cm to about 0.05 cm, about 0.001 cm to about 0.1 cm, about 0.001 cm to about 0.15 cm, about 0.001 cm to about 0.2 cm, about 0.001 cm to about 0.25 cm, about 0.001 cm to about 0.3 cm, about 0.001 cm to about 0.4 cm, about 0.001 cm to about 0.5 cm, about 0.005 cm to about 0.01 cm, about 0.005 cm to about 0.05 cm, about 0.005 cm to about 0.1 cm, about 0.005 cm to about 0.15 cm, about 0.005 cm to about 0.2 cm, about 0.005 cm to about 0.25 cm, about 0.005 cm to about 0.3 cm, about 0.005 cm to about 0.4 cm, about 0.005 cm to about 0.5 cm, about 0.01 cm to about 0.05 cm, about 0.01 cm to about 0.1 cm, about 0.01 cm to about 0.15 cm, about 0.01 cm to about 0.2 cm, about 0.01 cm to about 0.25 cm, about 0.01 cm to about 0.3 cm, about 0.01 cm to about 0.4 cm, about 0.01 cm to about 0.5 cm, about 0.05 cm to about 0.1 cm, about 0.05 cm to about 0.15 cm, about 0.05 cm to about 0.2 cm, about 0.05 cm to about 0.25 cm, about 0.05 cm to about 0.3 cm, about 0.05 cm to about 0.4 cm, about 0.05 cm to about 0.5 cm, about 0.1 cm to about 0.15 cm, about 0.1 cm to about 0.2 cm, about 0.1 cm to about 0.25 cm, about 0.1 cm to about 0.3 cm, about 0.1 cm to about 0.4 cm, about 0.1 cm to about 0.5 cm, about 0.15 cm to about 0.2 cm, about 0.15 cm to about 0.25 cm, about 0.15 cm to about 0.3 cm, about 0.15 cm to about 0.4 cm, about 0.15 cm to about 0.5 cm, about 0.2 cm to about 0.25 cm, about 0.2 cm to about 0.3 cm, about 0.2 cm to about 0.4 cm, about 0.2 cm to about 0.5 cm, about 0.25 cm to about 0.3 cm, about 0.25 cm to about 0.4 cm, about 0.25 cm to about 0.5 cm, about 0.3 cm to about 0.4 cm, about 0.3 cm to about 0.5 cm, or about 0.4 cm to about 0.5 cm. In some embodiments, the diameter of each of the fenestrations 45 ranges from about 0.001 cm, about 0.005 cm, about 0.01 cm, about 0.05 cm, about 0.1 cm, about 0.15 cm, about 0.2 cm, about 0.25 cm, about 0.3 cm, about 0.4 cm, or about 0.5 cm. In some embodiments, the diameter of each of the fenestrations 45 ranges from at least about 0.001 cm, about 0.005 cm, about 0.01 cm, about 0.05 cm, about 0.1 cm, about 0.15 cm, about 0.2 cm, about 0.25 cm, about 0.3 cm, or about 0.4 cm. In some embodiments, the diameter of each of the fenestrations 45 ranges from at most about 0.005 cm, about 0.01 cm, about 0.05 cm, about 0.1 cm, about 0.15 cm, about 0.2 cm, about 0.25 cm, about 0.3 cm, about 0.4 cm, or about 0.5 cm.

In some embodiments, the fenestrations are directionally biased to help promote better ablation medium coverage. In some instances, the fenestrations are crescent-shaped. In some instances, the crescent-shaped fenestrations are configured to direct the ablation medium across the outer surface 90 of the catheter. In some instances, the fenestration patterns help focus the ablation medium towards the neck of the gallbladder and access site to ensure proper coverage. In some embodiments, the fenestrated catheter nozzle 44 is configured to aerosolize an ablation medium. In some embodiments, the fenestrated catheter nozzle 44 is configured to aerosolize a cryogen. In some embodiments, the fenestrated catheter nozzle 44 is configured to aerosolize a thermal medium. In some instances, the fenestrated catheter nozzle 44 is configured to aerosolize a liquid medium. In some instances, the fenestrated catheter nozzle 44 is configured to aerosolize liquid nitrogen. In some instances, the fenestrated catheter nozzle 44 is configured to aerosolize liquid nitrous oxide. In some instances, the fenestrated catheter nozzle 44 is configured to aerosolize hot water.

In some embodiments, as shown in FIG. 7B, the catheter 66 comprises a nozzle exposure sheath 46. In some embodiments, the nozzle exposure sheath 46 has an inner diameter that is equal to or slightly greater than the outer diameter of the catheter 66, which allows the nozzle exposure sheath 46 to be slidably positioned along the outer surface 90 of the catheter. In some embodiments, the nozzle exposure sheath 46 is advanced, slidably positioned, or any combination thereof over the fenestrations 45 in order to close a predetermined length, area, or any combination thereof of the fenestrated nozzle 44 and optionally leave an exposed length area, or any combination thereof of the fenestrated nozzle 44 uncovered or exposed in order to dispense an ablation medium (e.g., a fluid, a gas, or any combination thereof) across the outer surface 90 of the catheter. In some embodiments, the nozzle exposure sheath 46 is advanced, slidably positioned, or any combination thereof over the fenestrations 45 in the direction of the arrows shown in FIG. 7B. In some embodiments, the nozzle exposure sheath 46 has a length which is greater than the length of the fenestrated nozzle 44. In some embodiments, the nozzle exposure sheath 46 and the outer surface 90 of the catheter is composed of a material with a low coefficient of friction, which allows the nozzle exposure sheath 46 to easily slide along the outer surface 90. Alternatively, in some instances, the nozzle exposure sheath 46 and the outer surface 90 are coated with a lubricious material, which allows the nozzle exposure sheath 46 to easily slide along the outer surface 90. In some embodiments, the nozzle exposure sheath 46 has one or more radiopaque markers, coatings, or any combination thereof (not shown in FIG. 7B) to aid in the visualization of the nozzle exposure sheath 46 via computer tomography (CT) or X-ray, for example.

In some instances, the nozzle exposure sheath 46 limits the flow of the ablation medium (e.g., a fluid, a gas, or any combination thereof) from the covered fenestrations. In some instances, the nozzle exposure sheath 46 prevents flow of the medium or fluid from the covered fenestrations. In some instances, the nozzle exposure sheath 46 runs along the inner diameter of the catheter. For nonlimiting example, an embodiment device comprising a fenestrated lumen is illustrated in FIG. 7A and an embodiment device comprising a fenestrated lumen with an adjustable nozzle exposure sheath 46 is on FIG. 7B.

In some embodiments, the nozzle exposure sheath 46 is attached to the device access sheath 6 on the proximal end. In some embodiments, the nozzle exposure sheath 46 is attached to the device access sheath 6. In some instances, a linear actuator is used to advance the nozzle exposure sheath 46 along the longitudinal axis of the catheter 66 to change the number of exposed fenestrations. In some instances, a linear actuator is used to retract the nozzle exposure sheath 46 along the longitudinal axis of the catheter 66 to change the number of exposed fenestrations. In some instances, the provider measures the size of the gallbladder and adjusts the nozzle exposure sheath 46 and catheter 66 to fit within the anatomy (i.e., the gallbladder lumen) and adjust the exposed fenestrated area of the catheter in order to achieve maximum ablation exposure.

In some embodiments, the nozzle exposure sheath 46 does not fully surround the catheter, leaving an open space through which ablation medium flows through. In some instances, the open space results in selective dispersion of the medium. In some embodiments, the nozzle exposure sheath 46 is non-concentric. In some instances, the nozzle exposure sheath 46 is has a rotational freedom of about 360 degrees around the longitudinal axis of the fenestrated nozzle 44, of the catheter 66, or any combination thereof, thereby allowing the preferential dispersion of ablation medium. In some embodiments, the position of the catheter is controlled by the device access sheath 6.

In an alternative embodiment, the catheter device 4 does not comprise a nozzle exposure sheath an instead, the size of the fenestrated nozzle 44 is varied. For example, in some embodiments, the size (e.g., the length, the area, the fenestration pattern, or a combination thereof) of the fenestrated nozzle 44 is varied according to the physiological measurements of the patient (e.g., the size of the gallbladder lumen). In yet another embodiment, the ablation catheter is retracted while ablating in order to control the amount of ablation medium delivered onto the outer surface or wall of the gallbladder.

In some embodiments, the device uniformly delivers an ablation medium to the mucosal surface of the gallbladder. In some instances, the device uniformly delivers an ablation medium to the mucosal surface of the gallbladder and facilitates occlusion of the cystic duct.

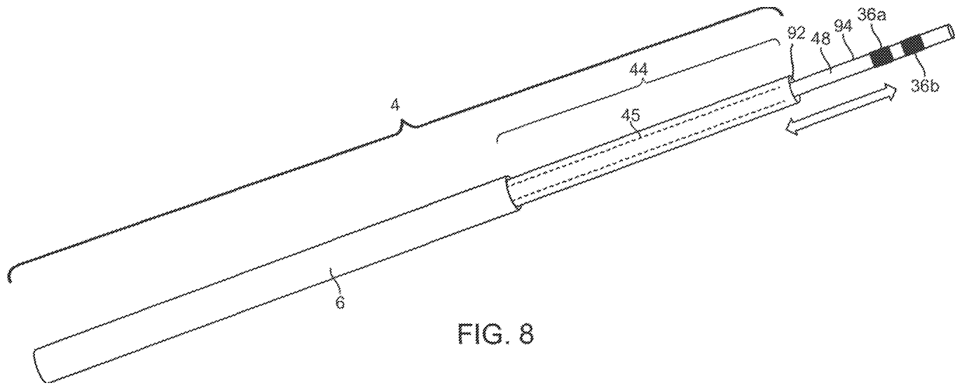

In some embodiments, the catheter device comprises an additional probe. In some embodiments, the catheter device comprises at least one radio frequency (RF) ablater 48, as shown in FIG. 8. In some embodiments, the RF ablater 48 comprises a first electrode 36a and a second electrode 36b. In some embodiments, the catheter 66 has an inner diameter that is equal to or slightly greater than the outer diameter of the RF ablater 48, which allows the catheter 66 to be slidably positioned along the outer surface 94 of the RF ablater 48. In some embodiments, the RF ablater 48 is advanced for a predetermined length. In some embodiments, the RF ablater 48 is advanced in the direction of the arrows shown in FIG. 8. In some embodiments, the RF ablater 48 has a length which is greater than the length of the fenestrated nozzle 44. In some embodiments, the catheter 66 and the outer surface 94 of the RF ablater 48 is composed of a material with a low coefficient of friction, which allows the RF ablater 48 to easily slide through the lumen 92 of the catheter. Alternatively, in some instances, the outer surface 94 of the RF ablater 48 and lumen 92 of the catheter are coated with a lubricious material, which allows the RF ablater 48 to easily slide through the lumen 92 of the catheter. In some embodiments, the RF ablater 48 has one or more radiopaque markers, coatings, or any combination thereof (not shown in FIG. 8) to aid in the visualization of the RF ablater 48 via computer tomography (CT) or X-ray, for example.

In some embodiments, the lumen 92 of the catheter helps facilitate the insertion of additional tools (e.g., additional probes, catheters, guidewires, or any combination thereof). In some instances, the lumen 92 facilitates the insertion of a radio frequency (RF) ablater 48 to help occlude the cystic duct of an individual in need thereof. Alternatively, in other instances, the lumen 92 facilitates the insertion of a cryogen ablater (not shown in the figures) to help occlude the cystic duct. In some instances, the lumen 92 is compatible with a standard guidewire to facilitate access to the cystic duct of an individual in need thereof. In some instances, the lumen 92 is concentric with the fenestrated nozzle 44, the catheter, or any combination thereof and the interstitial space between the lumen 92 and the fenestrated nozzle 44, the catheter, or any combination thereof allows for the flow of an ablation medium (e.g., a fluid, a gas, or any combination thereof).

In some embodiments, the catheter device provided herein is deflectable with a drive wire and the device access sheath 6 to bias the distal tip into the cystic duct of an individual in need thereof. In some instances, the deflection is actuated.

In some embodiments, the overall length of the elongated body of the catheter device is between 5 cm and 50 cm. In some embodiments, the overall length of the elongated body is at least 5 cm, 10 cm, 20 cm, 30 cm, 40 cm, 50 cm, 60 cm, 70 cm, 80 cm, 90 cm, or 100 cm. In some embodiments, the length of the catheter 66 ranges from about 5 cm to about 200 cm. In some embodiments, the length of the catheter 66 ranges from about 5 cm to about 10 cm, about 5 cm to about 20 cm, about 5 cm to about 30 cm, about 5 cm to about 40 cm, about 5 cm to about 50 cm, about 5 cm to about 60 cm, about 5 cm to about 70 cm, about 5 cm to about 80 cm, about 5 cm to about 90 cm, about 5 cm to about 100 cm, about 5 cm to about 200 cm, about 10 cm to about 20 cm, about 10 cm to about 30 cm, about 10 cm to about 40 cm, about 10 cm to about 50 cm, about 10 cm to about 60 cm, about 10 cm to about 70 cm, about 10 cm to about 80 cm, about 10 cm to about 90 cm, about 10 cm to about 100 cm, about 10 cm to about 200 cm, about 20 cm to about 30 cm, about 20 cm to about 40 cm, about 20 cm to about 50 cm, about 20 cm to about 60 cm, about 20 cm to about 70 cm, about 20 cm to about 80 cm, about 20 cm to about 90 cm, about 20 cm to about 100 cm, about 20 cm to about 200 cm, about 30 cm to about 40 cm, about 30 cm to about 50 cm, about 30 cm to about 60 cm, about 30 cm to about 70 cm, about 30 cm to about 80 cm, about 30 cm to about 90 cm, about 30 cm to about 100 cm, about 30 cm to about 200 cm, about 40 cm to about 50 cm, about 40 cm to about 60 cm, about 40 cm to about 70 cm, about 40 cm to about 80 cm, about 40 cm to about 90 cm, about 40 cm to about 100 cm, about 40 cm to about 200 cm, about 50 cm to about 60 cm, about 50 cm to about 70 cm, about 50 cm to about 80 cm, about 50 cm to about 90 cm, about 50 cm to about 100 cm, about 50 cm to about 200 cm, about 60 cm to about 70 cm, about 60 cm to about 80 cm, about 60 cm to about 90 cm, about 60 cm to about 100 cm, about 60 cm to about 200 cm, about 70 cm to about 80 cm, about 70 cm to about 90 cm, about 70 cm to about 100 cm, about 70 cm to about 200 cm, about 80 cm to about 90 cm, about 80 cm to about 100 cm, about 80 cm to about 200 cm, about 90 cm to about 100 cm, about 90 cm to about 200 cm, or about 100 cm to about 200 cm. In some embodiments, the length of the catheter 66 ranges from about 5 cm, about 10 cm, about 20 cm, about 30 cm, about 40 cm, about 50 cm, about 60 cm, about 70 cm, about 80 cm, about 90 cm, about 100 cm, or about 200 cm. In some embodiments, the length of the catheter 66 ranges from at least about 5 cm, about 10 cm, about 20 cm, about 30 cm, about 40 cm, about 50 cm, about 60 cm, about 70 cm, about 80 cm, about 90 cm, or about 100 cm. In some embodiments, the length of the catheter 66 ranges from at most about 10 cm, about 20 cm, about 30 cm, about 40 cm, about 50 cm, about 60 cm, about 70 cm, about 80 cm, about 90 cm, about 100 cm, or about 200 cm.

In some embodiments, the cross-sectional distance of the elongated body is between 0.5 mm and 5 mm. In some embodiments, the cross-sectional distance of the elongated body is at least 0.5 mm, 1 mm, 2 mm, 3 mm, 4 mm, or 5 mm. In some embodiments, the diameter of the catheter 66 ranges from about 0.1 mm to about 10 mm. In some embodiments, the diameter of the catheter 66 ranges from about 0.1 mm to about 0.5 mm, about 0.1 mm to about 1 mm, about 0.1 mm to about 2 mm, about 0.1 mm to about 3 mm, about 0.1 mm to about 4 mm, about 0.1 mm to about 5 mm, about 0.1 mm to about 10 mm, about 0.5 mm to about 1 mm, about 0.5 mm to about 2 mm, about 0.5 mm to about 3 mm, about 0.5 mm to about 4 mm, about 0.5 mm to about 5 mm, about 0.5 mm to about 10 mm, about 1 mm to about 2 mm, about 1 mm to about 3 mm, about 1 mm to about 4 mm, about 1 mm to about 5 mm, about 1 mm to about 10 mm, about 2 mm to about 3 mm, about 2 mm to about 4 mm, about 2 mm to about 5 mm, about 2 mm to about 10 mm, about 3 mm to about 4 mm, about 3 mm to about 5 mm, about 3 mm to about 10 mm, about 4 mm to about 5 mm, about 4 mm to about 10 mm, or about 5 mm to about 10 mm. In some embodiments, the diameter of the catheter 66 ranges from about 0.1 mm, about 0.5 mm, about 1 mm, about 2 mm, about 3 mm, about 4 mm, about 5 mm, or about 10 mm. In some embodiments, the diameter of the catheter 66 ranges from at least about 0.1 mm, about 0.5 mm, about 1 mm, about 2 mm, about 3 mm, about 4 mm, or about 5 mm. In some embodiments, the diameter of the catheter 66 ranges from at most about 0.5 mm, about 1 mm, about 2 mm, about 3 mm, about 4 mm, about 5 mm, or about 10 mm.

In some embodiments, the ablation balloon in the inflated configuration has a cross-sectional distance of at least 0.1 cm, 0.5 cm, 1 cm, 2 cm, 3 cm, 4 cm, or 5 cm. In some embodiments, the ablation balloon in the inflated configuration has a diameter ranging from about 0.1 cm to about 10 cm. In some embodiments, the ablation balloon in the inflated configuration has a diameter ranging from about 0.1 cm to about 0.5 cm, about 0.1 cm to about 1 cm, about 0.1 cm to about 2 cm, about 0.1 cm to about 3 cm, about 0.1 cm to about 4 cm, about 0.1 cm to about 5 cm, about 0.1 cm to about 10 cm, about 0.5 cm to about 1 cm, about 0.5 cm to about 2 cm, about 0.5 cm to about 3 cm, about 0.5 cm to about 4 cm, about 0.5 cm to about 5 cm, about 0.5 cm to about 10 cm, about 1 cm to about 2 cm, about 1 cm to about 3 cm, about 1 cm to about 4 cm, about 1 cm to about 5 cm, about 1 cm to about 10 cm, about 2 cm to about 3 cm, about 2 cm to about 4 cm, about 2 cm to about 5 cm, about 2 cm to about 10 cm, about 3 cm to about 4 cm, about 3 cm to about 5 cm, about 3 cm to about 10 cm, about 4 cm to about 5 cm, about 4 cm to about 10 cm, or about 5 cm to about 10 cm. In some embodiments, the ablation balloon in the inflated configuration has a diameter ranging from about 0.1 cm, about 0.5 cm, about 1 cm, about 2 cm, about 3 cm, about 4 cm, about 5 cm, or about 10 cm. In some embodiments, the ablation balloon in the inflated configuration has a diameter ranging from at least about 0.1 cm, about 0.5 cm, about 1 cm, about 2 cm, about 3 cm, about 4 cm, or about 5 cm. In some embodiments, the ablation balloon in the inflated configuration has a diameter ranging from at most about 0.5 cm, about 1 cm, about 2 cm, about 3 cm, about 4 cm, about 5 cm, or about 10 cm.

In some embodiments, the ablation balloon in the inflated configuration has a volume of at least 5 milliliters (ml), 10 ml, 20 ml, 30 ml, 40 ml, or 50 ml. In some embodiments, the ablation balloon in the inflated configuration has a volume ranging from about 1 ml to about 100 ml. In some embodiments, the ablation balloon in the inflated configuration has a volume ranging from about 1 ml to about 5 ml, about 1 ml to about 10 ml, about 1 ml to about 20 ml, about 1 ml to about 30 ml, about 1 ml to about 40 ml, about 1 ml to about 50 ml, about 1 ml to about 60 ml, about 1 ml to about 70 ml, about 1 ml to about 80 ml, about 1 ml to about 90 ml, about 1 ml to about 100 ml, about 5 ml to about 10 ml, about 5 ml to about 20 ml, about 5 ml to about 30 ml, about 5 ml to about 40 ml, about 5 ml to about 50 ml, about 5 ml to about 60 ml, about 5 ml to about 70 ml, about 5 ml to about 80 ml, about 5 ml to about 90 ml, about 5 ml to about 100 ml, about 10 ml to about 20 ml, about 10 ml to about 30 ml, about 10 ml to about 40 ml, about 10 ml to about 50 ml, about 10 ml to about 60 ml, about 10 ml to about 70 ml, about 10 ml to about 80 ml, about 10 ml to about 90 ml, about 10 ml to about 100 ml, about 20 ml to about 30 ml, about 20 ml to about 40 ml, about 20 ml to about 50 ml, about 20 ml to about 60 ml, about 20 ml to about 70 ml, about 20 ml to about 80 ml, about 20 ml to about 90 ml, about 20 ml to about 100 ml, about 30 ml to about 40 ml, about 30 ml to about 50 ml, about 30 ml to about 60 ml, about 30 ml to about 70 ml, about 30 ml to about 80 ml, about 30 ml to about 90 ml, about 30 ml to about 100 ml, about 40 ml to about 50 ml, about 40 ml to about 60 ml, about 40 ml to about 70 ml, about 40 ml to about 80 ml, about 40 ml to about 90 ml, about 40 ml to about 100 ml, about 50 ml to about 60 ml, about 50 ml to about 70 ml, about 50 ml to about 80 ml, about 50 ml to about 90 ml, about 50 ml to about 100 ml, about 60 ml to about 70 ml, about 60 ml to about 80 ml, about 60 ml to about 90 ml, about 60 ml to about 100 ml, about 70 ml to about 80 ml, about 70 ml to about 90 ml, about 70 ml to about 100 ml, about 80 ml to about 90 ml, about 80 ml to about 100 ml, or about 90 ml to about 100 ml. In some embodiments, the ablation balloon in the inflated configuration has a volume ranging from about 1 ml, about 5 ml, about 10 ml, about 20 ml, about 30 ml, about 40 ml, about 50 ml, about 60 ml, about 70 ml, about 80 ml, about 90 ml, or about 100 ml. In some embodiments, the ablation balloon in the inflated configuration has a volume ranging from at least about 1 ml, about 5 ml, about 10 ml, about 20 ml, about 30 ml, about 40 ml, about 50 ml, about 60 ml, about 70 ml, about 80 ml, or about 90 ml. In some embodiments, the ablation balloon in the inflated configuration has a volume ranging from at most about 5 ml, about 10 ml, about 20 ml, about 30 ml, about 40 ml, about 50 ml, about 60 ml, about 70 ml, about 80 ml, about 90 ml, or about 100 ml.

In some embodiments, the catheter device 4 comprises a catheter 66 having a size equivalent to that of a catheter between 1.5 French (Fr) and 15 Fr. In some embodiments, the catheter device 4 comprises a catheter 66 having a size ranging from about 1 Fr to about 15 Fr. In some embodiments, the catheter device 4 comprises a catheter 66 having a size ranging from about 1 Fr to about 1.5 Fr, about 1 Fr to about 2 Fr, about 1 Fr to about 3 Fr, about 1 Fr to about 4 Fr, about 1 Fr to about 5 Fr, about 1 Fr to about 6 Fr, about 1 Fr to about 7 Fr, about 1 Fr to about 8 Fr, about 1 Fr to about 9 Fr, about 1 Fr to about 10 Fr, about 1 Fr to about 15 Fr, about 1.5 Fr to about 2 Fr, about 1.5 Fr to about 3 Fr, about 1.5 Fr to about 4 Fr, about 1.5 Fr to about 5 Fr, about 1.5 Fr to about 6 Fr, about 1.5 Fr to about 7 Fr, about 1.5 Fr to about 8 Fr, about 1.5 Fr to about 9 Fr, about 1.5 Fr to about 10 Fr, about 1.5 Fr to about 15 Fr, about 2 Fr to about 3 Fr, about 2 Fr to about 4 Fr, about 2 Fr to about 5 Fr, about 2 Fr to about 6 Fr, about 2 Fr to about 7 Fr, about 2 Fr to about 8 Fr, about 2 Fr to about 9 Fr, about 2 Fr to about 10 Fr, about 2 Fr to about 15 Fr, about 3 Fr to about 4 Fr, about 3 Fr to about 5 Fr, about 3 Fr to about 6 Fr, about 3 Fr to about 7 Fr, about 3 Fr to about 8 Fr, about 3 Fr to about 9 Fr, about 3 Fr to about 10 Fr, about 3 Fr to about 15 Fr, about 4 Fr to about 5 Fr, about 4 Fr to about 6 Fr, about 4 Fr to about 7 Fr, about 4 Fr to about 8 Fr, about 4 Fr to about 9 Fr, about 4 Fr to about 10 Fr, about 4 Fr to about 15 Fr, about 5 Fr to about 6 Fr, about 5 Fr to about 7 Fr, about 5 Fr to about 8 Fr, about 5 Fr to about 9 Fr, about 5 Fr to about 10 Fr, about 5 Fr to about 15 Fr, about 6 Fr to about 7 Fr, about 6 Fr to about 8 Fr, about 6 Fr to about 9 Fr, about 6 Fr to about 10 Fr, about 6 Fr to about 15 Fr, about 7 Fr to about 8 Fr, about 7 Fr to about 9 Fr, about 7 Fr to about 10 Fr, about 7 Fr to about 15 Fr, about 8 Fr to about 9 Fr, about 8 Fr to about 10 Fr, about 8 Fr to about 15 Fr, about 9 Fr to about 10 Fr, about 9 Fr to about 15 Fr, or about 10 Fr to about 15 Fr. In some embodiments, the catheter device 4 comprises a catheter 66 having a size ranging from about 1 Fr, about 1.5 Fr, about 2 Fr, about 3 Fr, about 4 Fr, about 5 Fr, about 6 Fr, about 7 Fr, about 8 Fr, about 9 Fr, about 10 Fr, or about 15 Fr. In some embodiments, the catheter device 4 comprises a catheter 66 having a size ranging from at least about 1 Fr, about 1.5 Fr, about 2 Fr, about 3 Fr, about 4 Fr, about 5 Fr, about 6 Fr, about 7 Fr, about 8 Fr, about 9 Fr, or about 10 Fr. In some embodiments, the catheter device 4 comprises a catheter 66 having a size ranging from at most about 1.5 Fr, about 2 Fr, about 3 Fr, about 4 Fr, about 5 Fr, about 6 Fr, about 7 Fr, about 8 Fr, about 9 Fr, about 10 Fr, or about 15 Fr.

### Cystic Duct Occluder

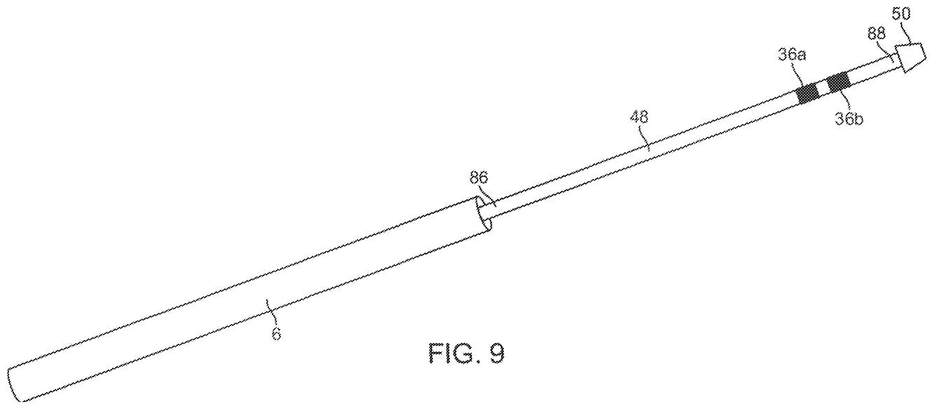

In some embodiments, the catheter device comprises a cystic duct occluder. In some embodiments, the cystic duct occluder comprises a plug 50, as shown in FIG. 9. In some embodiments, the plug 50 is a temporary occlusion plug that is to occlude the cystic duct of an individual in need thereof of a predetermined period of time. In some embodiments, the plug 50 is a permanent occlusion plug that is to permanently occlude the cystic duct of an individual in need thereof. In some embodiments, the cystic duct occluder comprises no temporary occlusion plugs. Alternatively, or in addition, the cystic duct occluder comprises a chronic occlusion plug. In some embodiments, the temporary occlusion plug is formed of a biodegradable or resorbable material. In some embodiments, the biodegradable or resorbable material is a polymer, a hydrogel, glue, an adhesive, or any combination thereof. In some embodiments, the plug 50 is coupled to the distal end 88 of the catheter 66. In some embodiments, the location of the plug 50 at the distal end 88 facilitates the targeting of the cystic duct of an individual in need thereof. In some embodiments, the plug is mechanically decoupled or ejected from the catheter 66 allowing the placement of the plug 50 in a desired anatomical location (e.g., a cystic duct) of an individual in need thereof. Next, after positioning the plug 50 in the desired anatomical location, the plug 50 is fixed in place via several methods, including but not limited to volume expansion of the plug, external threads, friction fit, adhesion, or any combination thereof.

In some embodiments, the plug 50 is made of a material that allows for a small gauge guidewire (e.g., a small gauge guidewire having a diameter of about 0.018 inches) to be placed through it and removed, without losing its occlusive properties. In other words, in some embodiments, the plug 50 is made of a re-sealable material, comprises a membrane made of a re-sealable material, or any combination thereof. In some embodiments, the re-sealable material is a thermoplastic elastomer. In some embodiments, the re-sealable material is polyvinyl chloride (PVC), styrenic block copolymer, thermoplastic polyolefinelastomer, thermoplastic vulcanizate, thermoplastic polyurethane, thermoplastic copolyester, thermoplastic polyamide, or any combination thereof.

In some embodiments, the plug 50 remains in place in a desired anatomical location (e.g., within a cystic duct) for at least two weeks to allow for chronic occlusion, or lasts indefinitely, or any period in between. In some embodiments, the plug 50 prevents bile from re-entering the gallbladder and reduces the likelihood of re-epithelialization of the mucosal layer of the gallbladder while chronic occlusion occurs.

In some embodiments, the catheter 66 comprises a first electrode 36a and a second electrode 36b. In some embodiments, the first electrode 36a and a second electrode 36b are bipolar RF electrodes, as described elsewhere herein. In some embodiments, the first electrode 36a and a second electrode 36b are located proximal to the plug 50, as shown in FIG. 9. In some embodiments, the first electrode 36a and a second electrode 36b are located at the distal end 88 of the catheter 66, as shown in FIG. 9. In some embodiments, the first electrode 36a and a second electrode 36b are located at the proximal end 86 of the catheter. In some embodiments, the first electrode 36a and a second electrode 36b are located at any location between the proximal end 86 and the distal end 88 of the catheter 66. In some embodiments, the chronic occlusion technique comprises a pair of bipolar RF electrodes located proximal to a temporary occlusion plug. In some embodiments, the first electrode 36a and a second electrode 36b are used to induce chronic scarring in the cystic duct at the neck of the gallbladder.

In some embodiments, a chronic occlusion technique is performed with a catheter comprising a fenestrated nozzle, a first electrode, a second electrode (as shown in FIG. 8), and a plug. In some embodiments, the catheter shown in FIG. 8 further comprises a plug (having the plug positioned as illustrated in FIG. 9). In some embodiments, a chronic occlusion technique is performed with a catheter comprising a fenestrated nozzle, a first electrode, a second electrode, a nozzle exposure sheath, and a plug. In some embodiments, the catheter shown in FIG. 8 further comprises a nozzle exposure sheath and a plug (having the plug positioned as illustrated in FIG. 9). In some embodiments, the catheter shown in FIG. 8 further comprises a nozzle exposure sheath.

In some embodiments, the chronic occlusion technique is performed by cryoablation, thermal ablation, or chemical ablation at a proximal location to the plug. In some aspects, the plug is an optional part of the device disclosed herein. In some embodiments, chronic occlusion technique forms a scar tissue in the cystic duct to block the opening of the cystic duct. In some embodiments, the chronic occlusion technique stimulates the healing response of the subject to occlude the cystic duct. In some embodiments, the chronic occlusion technique permanently occludes the cystic duct.

In some embodiments, the plug provides a physical barrier between the gallbladder and the cystic duct. In some embodiments, the plug is inserted through any of the access methods described herein. In some embodiments, a guidewire and an introducer catheter are used to locate and cannulate the cystic duct for plug deployment. In some embodiments, the plug deploys and fixes to the cystic duct via several methods. In some embodiments, the plug is folded into a catheter, and upon catheter sheath retraction, expand in place. In some embodiments, the plug is made from a hydrogel or expandable material that grows when exposed to a hydrating environment. In some embodiments, the expandable material is a water swellable polymer or a superexpandable polymer. Non-limiting examples of expandable materials include poly(acrylic acid), poly(acrylic acid-co-acrylamide), poly(acrylic acid) and sodium salt-graft-poly(ethylene oxide), poly(2-hydroxyethyl methacrylate), poly(2-hydroxypropyl methacrylate), poly(isobutylene-co-maleic acid), ethylene maleic anhydride copolymer, cross-linked carboxymethylcellulose, polyvinyl alcohol copolymer, cross-linked polyethylene oxide, starch grafted copolymer of polyacrylonitrile, or any combination thereof.

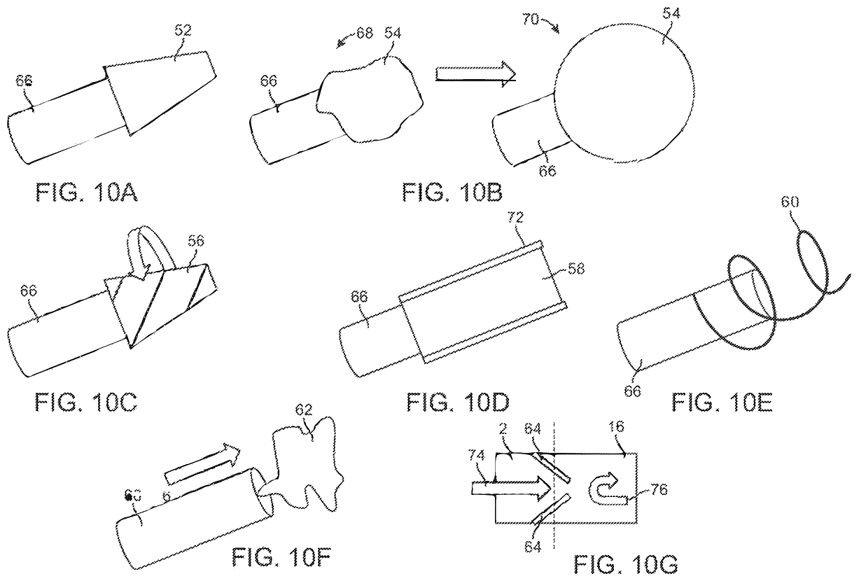

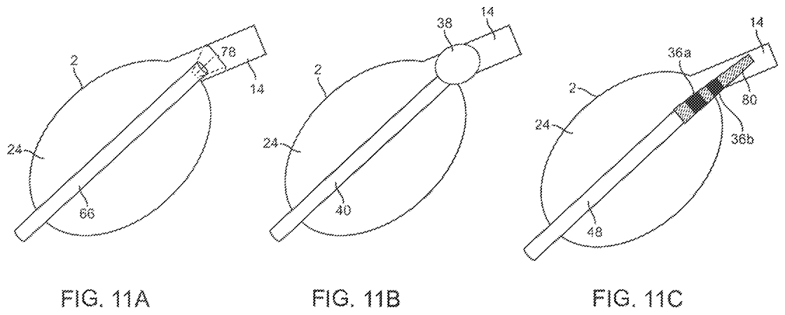

In some embodiments, the plug is a tapered plug 52, as shown in FIG. 10A. In some embodiments, the tapered plug 52 is tapered and wedges into the cystic duct by frictional force. In some embodiments, the plug is an inflatable plug 54 that is switched from an deflated state 68 to an inflated state 70, as shown in FIG. 10B. In some embodiments, the inflatable plug 54 comprises an inflatable balloon with concentric ridges to help improve stabilization. In some embodiments, the inflatable plug 54 is inflated with a gas, a liquid, or any combination thereof. In some embodiments, the plug is a threaded plug 56, as shown in FIG. 10C. In some embodiments, the threaded plug 56 comprises a one or more external threads and is configured to twist into a cystic duct. In some embodiments, the threaded plug is a threaded cylinder configured to be threaded into surrounding tissue (e.g., of the cystic duct) of a patient, thus providing a tight seal between the plug and the tissue.

In some embodiments, the plug is a tissue ingrowth plug 58, as shown in FIG. 10D. In some embodiments, the tissue ingrowth plug 58 comprises a profibrotic surface 72. In some embodiments, the tissue ingrowth plug 58 is made from a bioresorbable, dissolvable, or biodegradable material, such as, but not limited to polyglycolic acid (PGA), polylactic acid (PLA), polylactic-co-glycolic acid (PLGA), a proteoglycan, or any combination thereof. In some embodiments, the tissue ingrowth plug 58 is bioresorbable or biodegradable. In some embodiments, the tissue ingrowth plug 58 comprises a tissue ingrowth segment, which promotes securement via an immune response (i.e., through inflammation, scarring, or any combination thereof). In some embodiments, the tissue ingrowth segment acts as an anchoring portion to prevent premature dislodgment of the plug in both a chronic implant (i.e., a permanent occlusion plug) and a dissolvable material scenario (i.e., a temporary occlusion plug). In some embodiments, the profibrotic surface 72 comprises a profibrotic material, a profibrotic agent, or any combination thereof. In some embodiments, the profibrotic surface 72 comprises a synthetic mesh. For example, in some embodiments, the synthetic mesh is a permanent or an absorbable mesh. In some embodiments, the permanent mesh is a polypropylene mesh, a polyester mesh, an expanded polytetrafluoroethylene (ePTFE) mesh, or any combination thereof. In some embodiments, the absorbable mesh is a Dexon mesh, a Vicryl mesh, or any combination thereof. In some embodiments, the profibrotic material, profibrotic agent, or any combination thereof is transforming growth factor-beta (TGF-β), TGF-β1, methotrexate (MTX), thioacetamide (TAA), polypropylene, polyester, expanded polytetrafluoroethylene (ePTFE), polyglycolic acid (PGA), polylactic acid (PLA), polylactic-co-glycolic acid (PLGA), polyglactin 910, or any combination thereof.

In some embodiments, the plug is a coil plug 60, as shown in FIG. 10E. In In some embodiments, the coil plug 60 comprises a coil, a mesh, a stent, or any combination thereof that promotes embolization or ingrowth in the cystic duct lumen, acts as a lithogenic agent to help form cholesterol deposits on its structure, or any combination thereof. In some embodiments, the coil plug 60 localizes cholesterol to build a natural barrier in the duct. In some embodiments, the coil, mesh, stent, or any combination thereof are composed of a metal, a metal alloy, a plastic, or any combination thereof. In some embodiments, the metal alloy is nitinol, cobalt-chromium alloy, magnesium alloy, or any combination thereof. In some embodiments, the metal is stainless steel, tantalum, or any combination thereof. In some embodiments, the coil, mesh, stent, or any combination thereof comprise drug-eluding materials. In some embodiments, the coil, mesh, stent, or any combination thereof are coated with a material, an agent, or any combination thereof. In some embodiments, the agent is a profibrotic agent, an anti-inflammatory drug, an antibiotic drug, a scar-inducing agent, an inflammatory-inducing agent, or any combination thereof. In some embodiments, the material is silicon carbide, carbon, titanium-nitride-oxide, or any combination thereof.

In some embodiments, the plug is an adhesive plug 62, as shown in FIG. 10F. In some embodiments, the adhesive plug 62 comprises an adhesive, glue, gel, hydratable matrix, hydrogel, or any combination thereof. In some embodiments, the adhesive plug 62 is loaded into the end of a catheter 66 and injected into the cystic duct. In some embodiments, a mushroom cap geometry is used to contain the glue and prevent migration into the common bile duct. In some embodiments, the mushroom cap (not shown in the figures) is made from a dissolvable material and integrates into the adhesive. In some embodiments, the mushroom cap is made from a dissolvable material and integrates into the adhesive.

In some embodiments, the plug is a one-way valve plug 64, as shown in FIG. 10G. In some embodiments, the cystic duct occluder comprises a valve that is inserted into the cystic duct in order to preferentially regulate the flow of bile/mucus to and from the gallbladder 2 and the common bile duct 16. In some embodiments, when the one-way valve plug 64 is in a closed configuration, as shown in FIG. 10G, the bile, mucus, or any combination thereof originating from the gallbladder 2 (direction of flow from gallbladder depicted by arrow 74) enters the common bile duct 16. On the other hand, in some embodiments, when the one-way valve plug 64 is in a closed configuration, as shown in FIG. 10G, the bile, mucus, or any combination thereof originating from the common bile duct 16 (direction of flow from the common bile duct depicted by arrow 76) does enter the gallbladder 2. In some embodiments, the valve contains an inner valve portion which has a closed resting state, at which a known fluid pressure activates unilateral flow (i.e., bile or any other fluid does not flow into the gallbladder 2, but mucus flow out of the gallbladder 2 and into the common bile duct 16). In some embodiments, the valve is a ball valve, check valve, or duckbill valve. In some embodiments, the valve is made from a fixed or multi-durometer polymer. In some embodiments, the external portion of the valve is fixed to prevent the valve from migrating into the cystic duct. In some embodiments, the valve is fixed to the surrounding tissue via an external thread, an adhesive glue, a tissue ingrowth promoting material, a tapered surface, a spiked or tined surface, a highly contouring surface, or any combination thereof.

FIGS. 11A, 11B, and 11C illustrate exemplary permanent cystic duct occluders that the catheter devices disclosed herein provide. In some embodiments, the cystic duct occluder is used in combination with the ablation delivery system provided herein. In some embodiments, the cystic duct occluder is used on a patient on the same day as the ablation delivery system is used on a patient. In some embodiments, the cystic duct occluder is used on a patient before the ablation delivery system is used on a patient. In some embodiments, the cystic duct occluder is used on a patient after the ablation delivery system is used on a patient. In some embodiments, the cystic duct occluder is delivered or applied to a patient after a determined period of time after the ablation delivery system is used on a patient. In some embodiments, the determined period of time is at least about 1 hour, 1 day, 2 days, 3 days, 1 week, 2 weeks, 3 weeks, 1 month, 6 months, 1 year, 5 years or more. In some embodiments, the cystic duct occluder is delivered or applied to a patient after the removal of gallstones from the gallbladder. In some embodiments, the cystic duct occluder is delivered or applied to a patient before the removal of gallstones from the gallbladder. In some embodiments, the cystic duct occluder is delivered or applied to a patient after the gallbladder is ablated. In some embodiments, the cystic duct occluder is delivered or applied to a patient before the gallbladder is ablated.

FIG. 11A illustrate an occluder that is a cystic duct ablation medium. In some embodiments, the cystic duct occluder is a cystic duct ablation medium that is sprayed through an opening of a catheter. In some embodiments, the cystic duct occluder is a cystic duct ablation medium that is sprayed through a fenestrated catheter. In some embodiments, the cystic duct occluder is a cystic duct ablation medium that is sprayed through a fenestrated ablation balloon. In some embodiments, the catheter device comprises a cystic duct occluder comprising a cystic duct ablation medium (e.g., a cryogen) that is delivered, sprayed, applied, or any combination thereof in the desired zone of ablation (i.e., into the cystic duct 14), as shown in FIG. 11A. In some embodiments, the cystic duct occluder prevents a gallbladder ablation medium delivered to the lumen of a gallbladder from migrating into other anatomic structures and preventing unintended damage thereto. In some instances, the catheter device delivers a first cystic duct ablation medium and a second cystic duct ablation medium in the desired zone of ablation (i.e., into the cystic duct 14). In some instances, the catheter device prevents the first cystic duct ablation medium and the second cystic duct ablation medium from migrating into other anatomic structures and preventing unintended damage thereto.

In some embodiments, the catheter 66 comprises an ablation medium delivery system that generates an ablation medium spray 78, which is directed at a cystic duct. In some embodiments, the ablation medium delivery system is a fenestrated nozzle, a nozzle exposure sheath, or any combination thereof. In some embodiments, the ablation medium delivery system is a sprayer, a spray applicator, an irrigator, or any combination thereof. In some embodiments, the ablation medium delivery system comprises a fluid transfer pump. In some embodiments, the ablation medium delivery system is an open lumen of the catheter through which the cystic duct ablation medium can flow through and exit the catheter. In some embodiments, the catheter device further comprises a fluid transfer pump that is used to transfer a cystic duct ablation medium from an extracorporeal reservoir into a cystic duct tissue of a patient via the catheter. In some embodiments, the fluid transfer pump causes a cystic duct ablation medium to be expelled by the fenestrated catheter nozzle, from the lumen of the fenestrated catheter nozzle, across the outer surface of the catheter. In some embodiments, the fluid transfer pump causes a cystic duct ablation medium to be expelled by a sprayer, a spray applicator, an irrigator, or any combination thereof from the lumen of the catheter into a surrounding cystic duct tissue of the patient.

In some embodiments, a heated conductive ablation medium is circulated through the inner lumen of the catheter in order to conductively ablate the surrounding tissue. In some instances, a cold conductive ablation medium is circulated through the inner lumen of the catheter in order to conductively ablate the surrounding tissue. In some embodiments, the catheter is circumferentially perforated in order for a heated ablation medium to be dispersed into the cystic duct. In some embodiments, the catheter is circumferentially perforated in order for a cold ablation medium to be dispersed into the cystic duct.

In some embodiments, alternatively, or in combination with the cystic duct occluder, mucosal ablation of the gallbladder is performed. In some embodiments, mucosal ablation of the gallbladder and mucosal ablation of the cystic duct are performed in combination. In some embodiments, mucosal ablation of the gallbladder and mucosal ablation of the cystic duct are performed on the same day. In some embodiments, mucosal ablation of the gallbladder and mucosal ablation of the cystic duct are performed on a different day. In some embodiments, the mucosal ablation of the cystic duct, the mucosal ablation of the gallbladder, or any combination thereof is performed by delivering an ablation medium via a separate catheter that slips over a catheter used to deliver the plug for cystic duct occlusion. In some embodiments, the catheter device comprises one or more catheters that enable the user to vary the location of the ablation independently of the location of the cystic duct occluder and accommodate for subject variability.

FIG. 11B illustrates an exemplary cystic duct occluder provided by the catheter devices disclosed herein. In some embodiments, the cystic duct occluder comprises an ablation balloon catheter 40 further comprising an ablation balloon 38. In some embodiments, the ablation balloon 38 disclosed herein is spherical. In some instances, the ablation balloon 38 disclosed herein is conical. In some instances, the ablation balloon 38 disclosed herein is cylindrical. For non-limiting example, an embodiment of an ablation balloon 38 located in a cystic duct 14 is shown in FIG. 11B.

In some embodiments, the ablation balloon 38 disclosed herein has radiopaque markers to aid in visualization. In some instances, the ablation balloon 38 is embedded with hyperechoic markers, such as microbubbles. In some instances, the ablation balloon 38 is embedded with hyperechoic markers, such as reflective nanoparticles.

In some embodiments, the ablation balloon 38 disclosed herein is comprised of silicone, polyurethane, other compliant polymers, or any combination thereof. In some instances, the ablation balloon 38 is inflated with air. In some instances, the ablation balloon 38 is inflated with water. In some instances, the ablation balloon is inflated 38 with saline. In some instances, the ablation balloon 38 is inflated with water. In some instances, the ablation balloon is inflated 38 with glycerin. In some instances, the ablation balloon 38 is inflated with water. In some instances, the ablation balloon 38 is inflated with saline, water, air, glycerin, a cryogen, a thermal ablation medium, dextrose, or any combination thereof. In some instances, the ablation balloon 38 is inflated with any other suitable medium known by the skilled artisan.

In some embodiments, the ablation balloon 38 disclosed herein includes a temperature sensor that is embedded into the walls of the ablation balloon 38. In yet another embodiment, the temperature sensor is located at the neck of the ablation balloon 38. In some instances, the ablation balloon 38 includes a pressure sensor that is embedded into the walls of the ablation balloon 38. In yet another embodiment, the pressure sensor is located at the neck of the ablation balloon 38. In some embodiments, the temperature sensor, the pressure sensor, or any combination thereof are removably located in the ablation balloon 38 or are removably connected to the ablation balloon 38. For example, in some embodiments, the temperature sensor, the pressure sensor, or any combination thereof are introduced into the lumen of the ablation balloon 38 via the catheter.

In some embodiments, the temperature sensor provides feedback to the extracorporeal control unit in order to complete a feedback loop that controls mucosal ablation. In some instances, the pressure sensor provides feedback to the extracorporeal control unit in order to complete a feedback loop that controls mucosal ablation.

In some embodiments, the cystic duct occluder disclosed herein is an ablation balloon located on the distal end of the catheter. In some instances, the ablation balloon is navigated into the cystic duct under fluoroscopic guidance. In some instances, the ablation balloon is navigated into the cystic duct under ultrasound guidance. In some instances, the ablation balloon is navigated into the cystic duct under direct visualization. In some instances, the balloon is inflated until opposition to the cystic duct lumen is achieved. In some embodiments, the ablation balloon is referred to as cystic duct distal balloon.

FIG. 11C illustrates yet another example of a cystic duct occluder provided by the catheter devices disclosed herein. In some embodiments, the RF ablater 48 is used to ablate the cystic duct and induce chronic scarring, thereby providing a permanent occlusion of the cystic duct. In some embodiments, the cystic duct occluder comprises a radiofrequency (RF) ablater 48 whereby the distal end of the RF ablater 48 tapers to an outer diameter that is sufficiently small to fit within the cystic duct 14, but large enough that the device opposes all walls of the cystic duct, creating a seal which prevents the passage of the gallbladder ablation medium. For non-limiting example, an embodiment of a tapered tip 80 is illustrated in FIG. 11C. In some embodiments, the tapered tip 80 is a suction tapered tip.

In some embodiments, the cystic duct occluder comprises an elongated tapered end that is delivered sufficiently far into the cystic duct to create a seal. In some instances, the catheter has a broad shaped terminus that seats against the narrow neck region of the gallbladder with a nipple-like protrusion that occupies the cystic duct. In some embodiments, the ablation medium is extruded from this tapered tip 80, promoting ablation by direct contact.

In some embodiments, the cystic duct occluder is an RF ablater 48 comprising a first electrode 36a and a second electrode 36b that induce ablation by RF ablation, as seen in FIG. 11C. In some embodiments, the RF ablater 48 is energized to deliver heat, ablate, and consequently induce tissue necrosis in the tissue that comes in contact with the RF ablater 48 (e.g., the cystic duct).

In some embodiments, there are at least two bipolar RF electrodes along the elongated body of the RF ablater 48. In some embodiments, the RF electrodes are spaced apart by 2 mm. In some embodiments, the bipolar RF electrodes are spaced apart by at least 0.5 mm, 1 mm, 2 mm, 3 mm, 4 mm, 5 mm, 6 mm, 7 mm, 8 mm, 9 mm, or 10 mm. In some embodiments, the RF electrodes are spaced apart by about 0.5 mm to about 20 mm. In some embodiments, the RF electrodes are spaced apart by about 0.5 mm to about 1 mm, about 0.5 mm to about 1.5 mm, about 0.5 mm to about 2 mm, about 0.5 mm to about 2.5 mm, about 0.5 mm to about 3 mm, about 0.5 mm to about 3.5 mm, about 0.5 mm to about 4 mm, about 0.5 mm to about 4.5 mm, about 0.5 mm to about 5 mm, about 0.5 mm to about 10 mm, about 0.5 mm to about 20 mm, about 1 mm to about 1.5 mm, about 1 mm to about 2 mm, about 1 mm to about 2.5 mm, about 1 mm to about 3 mm, about 1 mm to about 3.5 mm, about 1 mm to about 4 mm, about 1 mm to about 4.5 mm, about 1 mm to about 5 mm, about 1 mm to about 10 mm, about 1 mm to about 20 mm, about 1.5 mm to about 2 mm, about 1.5 mm to about 2.5 mm, about 1.5 mm to about 3 mm, about 1.5 mm to about 3.5 mm, about 1.5 mm to about 4 mm, about 1.5 mm to about 4.5 mm, about 1.5 mm to about 5 mm, about 1.5 mm to about 10 mm, about 1.5 mm to about 20 mm, about 2 mm to about 2.5 mm, about 2 mm to about 3 mm, about 2 mm to about 3.5 mm, about 2 mm to about 4 mm, about 2 mm to about 4.5 mm, about 2 mm to about 5 mm, about 2 mm to about 10 mm, about 2 mm to about 20 mm, about 2.5 mm to about 3 mm, about 2.5 mm to about 3.5 mm, about 2.5 mm to about 4 mm, about 2.5 mm to about 4.5 mm, about 2.5 mm to about 5 mm, about 2.5 mm to about 10 mm, about 2.5 mm to about 20 mm, about 3 mm to about 3.5 mm, about 3 mm to about 4 mm, about 3 mm to about 4.5 mm, about 3 mm to about 5 mm, about 3 mm to about 10 mm, about 3 mm to about 20 mm, about 3.5 mm to about 4 mm, about 3.5 mm to about 4.5 mm, about 3.5 mm to about 5 mm, about 3.5 mm to about 10 mm, about 3.5 mm to about 20 mm, about 4 mm to about 4.5 mm, about 4 mm to about 5 mm, about 4 mm to about 10 mm, about 4 mm to about 20 mm, about 4.5 mm to about 5 mm, about 4.5 mm to about 10 mm, about 4.5 mm to about 20 mm, about 5 mm to about 10 mm, about 5 mm to about 20 mm, or about 10 mm to about 20 mm. In some embodiments, the RF electrodes are spaced apart by about 0.5 mm, about 1 mm, about 1.5 mm, about 2 mm, about 2.5 mm, about 3 mm, about 3.5 mm, about 4 mm, about 4.5 mm, about 5 mm, about 10 mm, or about 20 mm. In some embodiments, the RF electrodes are spaced apart by at least about 0.5 mm, about 1 mm, about 1.5 mm, about 2 mm, about 2.5 mm, about 3 mm, about 3.5 mm, about 4 mm, about 4.5 mm, about 5 mm, or about 10 mm. In some embodiments, the RF electrodes are spaced apart by at most about 1 mm, about 1.5 mm, about 2 mm, about 2.5 mm, about 3 mm, about 3.5 mm, about 4 mm, about 4.5 mm, about 5 mm, about 10 mm, or about 20 mm. In some embodiments, there is at least one unipolar or monopolar RF electrodes. In some embodiments, there are a plurality of unipolar or monopolar RF electrodes.

In some embodiments, the RF for RF ablation is delivered for a predetermined amount of time. In some embodiments, the RF is delivered for at least 1 second, 5 seconds, 10 seconds, 15 seconds, 20 seconds, 25 seconds, 30 seconds, 35 seconds, 40 seconds, 45 seconds, 50 seconds, 55 seconds, or 60 seconds. In some embodiments, the RF is delivered for at least 1 minute, 5 minutes, 10 minutes, 15 minutes, 20 minutes, 25 minutes, or 30 minutes. In some embodiments, the RF is delivered for about 1 second to about 3,600 seconds. In some embodiments, the RF is delivered for about 1 second to about 5 seconds, about 1 second to about 15 seconds, about 1 second to about 30 seconds, about 1 second to about 45 seconds, about 1 second to about 60 seconds, about 1 second to about 120 seconds, about 1 second to about 300 seconds, about 1 second to about 600 seconds, about 1 second to about 900 seconds, about 1 second to about 1,800 seconds, about 1 second to about 3,600 seconds, about 5 seconds to about 15 seconds, about 5 seconds to about 30 seconds, about 5 seconds to about 45 seconds, about 5 seconds to about 60 seconds, about 5 seconds to about 120 seconds, about 5 seconds to about 300 seconds, about 5 seconds to about 600 seconds, about 5 seconds to about 900 seconds, about 5 seconds to about 1,800 seconds, about 5 seconds to about 3,600 seconds, about 15 seconds to about 30 seconds, about 15 seconds to about 45 seconds, about 15 seconds to about 60 seconds, about 15 seconds to about 120 seconds, about 15 seconds to about 300 seconds, about 15 seconds to about 600 seconds, about 15 seconds to about 900 seconds, about 15 seconds to about 1,800 seconds, about 15 seconds to about 3,600 seconds, about 30 seconds to about 45 seconds, about 30 seconds to about 60 seconds, about 30 seconds to about 120 seconds, about 30 seconds to about 300 seconds, about 30 seconds to about 600 seconds, about 30 seconds to about 900 seconds, about 30 seconds to about 1,800 seconds, about 30 seconds to about 3,600 seconds, about 45 seconds to about 60 seconds, about 45 seconds to about 120 seconds, about 45 seconds to about 300 seconds, about 45 seconds to about 600 seconds, about 45 seconds to about 900 seconds, about 45 seconds to about 1,800 seconds, about 45 seconds to about 3,600 seconds, about 60 seconds to about 120 seconds, about 60 seconds to about 300 seconds, about 60 seconds to about 600 seconds, about 60 seconds to about 900 seconds, about 60 seconds to about 1,800 seconds, about 60 seconds to about 3,600 seconds, about 120 seconds to about 300 seconds, about 120 seconds to about 600 seconds, about 120 seconds to about 900 seconds, about 120 seconds to about 1,800 seconds, about 120 seconds to about 3,600 seconds, about 300 seconds to about 600 seconds, about 300 seconds to about 900 seconds, about 300 seconds to about 1,800 seconds, about 300 seconds to about 3,600 seconds, about 600 seconds to about 900 seconds, about 600 seconds to about 1,800 seconds, about 600 seconds to about 3,600 seconds, about 900 seconds to about 1,800 seconds, about 900 seconds to about 3,600 seconds, or about 1,800 seconds to about 3,600 seconds. In some embodiments, the RF is delivered for about 1 second, about 5 seconds, about 15 seconds, about 30 seconds, about 45 seconds, about 60 seconds, about 120 seconds, about 300 seconds, about 600 seconds, about 900 seconds, about 1,800 seconds, or about 3,600 seconds. In some embodiments, the RF is delivered for at least about 1 second, about 5 seconds, about 15 seconds, about 30 seconds, about 45 seconds, about 60 seconds, about 120 seconds, about 300 seconds, about 600 seconds, about 900 seconds, or about 1,800 seconds. In some embodiments, the RF is delivered for at most about 5 seconds, about 15 seconds, about 30 seconds, about 45 seconds, about 60 seconds, about 120 seconds, about 300 seconds, about 600 seconds, about 900 seconds, about 1,800 seconds, or about 3,600 seconds.

In some embodiments, the RF is delivered at a power of at least 20 Watts (W), 40 W, 60 W, 80 W, or 100 W. In some embodiments, the RF is delivered at a power of about 10 W to about 500 W. In some embodiments, the RF is delivered at a power of about 10 W to about 20 W, about 10 W to about 40 W, about 10 W to about 60 W, about 10 W to about 80 W, about 10 W to about 100 W, about 10 W to about 200 W, about 10 W to about 500 W, about 20 W to about 40 W, about 20 W to about 60 W, about 20 W to about 80 W, about 20 W to about 100 W, about 20 W to about 200 W, about 20 W to about 500 W, about 40 W to about 60 W, about 40 W to about 80 W, about 40 W to about 100 W, about 40 W to about 200 W, about 40 W to about 500 W, about 60 W to about 80 W, about 60 W to about 100 W, about 60 W to about 200 W, about 60 W to about 500 W, about 80 W to about 100 W, about 80 W to about 200 W, about 80 W to about 500 W, about 100 W to about 200 W, about 100 W to about 500 W, or about 200 W to about 500 W. In some embodiments, the RF is delivered at a power of about 10 W, about 20 W, about 40 W, about 60 W, about 80 W, about 100 W, about 200 W, or about 500 W. In some embodiments, the RF is delivered at a power of at least about 10 W, about 20 W, about 40 W, about 60 W, about 80 W, about 100 W, or about 200 W. In some embodiments, the RF is delivered at a power of at most about 20 W, about 40 W, about 60 W, about 80 W, about 100 W, about 200 W, or about 500 W.

In some embodiments, the center of the cystic duct occluder is hollow, wherein a guidewire is able to pass through it. In some instances, the center of cystic duct occluder is hollow, wherein a small diameter catheter is able to pass through it.

In some embodiments, the distal tip of the RF ablater 48 has radiopaque markers to aid in visualization. In some instances, the catheter is embedded with hyperechoic markers, such as microbubbles. In some instances, the catheter is embedded with hyperechoic markers, such as reflective nanoparticles.

In some embodiments, the cystic duct occluder is a temporary cystic duct occluder. In some embodiments, the temporary cystic duct occluder temporarily occludes the cystic duct for a determined period of time. In some embodiments, the temporary cystic duct occluder comprises a cystic duct plug (not shown in FIGS. 11A, 11B, and 11C). In some embodiments, the cystic duct plug fits within the lumen of the cystic duct. In some embodiments, the cystic duct plug blocks the flow of bile through the cystic duct. In some embodiments, the plug is a bioabsorbable plug. In some embodiments, the plug is a non-bioabsorbable plug. In some embodiments, the plug comprises a biocompatible material. The plug comprises one or more medical grade materials. In some embodiments, the bioabsorbable plug comprises hydrogels, polymers, composites, or combinations thereof. In some embodiments, the plug expands after delivery to the lumen of the cystic duct to block the cystic duct. In some embodiments, the plug dissolves or degrades completely after 1 day, 3 days, 5 days, 1 week, 2 weeks, 3 week, or 4 weeks. In some aspects, the plug is an optional part of the catheter device disclosed herein.

### Computer Control Systems

The present disclosure provides computer control systems that are programmed to implement methods of the disclosure. FIG. 12 shows a computer system 101 that is programmed or otherwise configured to activate or de-activate ablater and ablation delivery systems of the catheter devices provided herein. In some embodiments, the computer system 101 regulates various aspects of the catheter device of the present disclosure, such as, for example, mechanically deploying, advancing, and retracting a catheter, an RF ablater, or any combination thereof; inflating and deflating an ablation balloon; controlling RF delivery pulses; controlling the temperature of an ablation medium; controlling the delivery of an ablation medium; controlling the active or passive evacuation flow rate of an ablation medium, controlling the supply flow rate of the ablation medium, and controlling the position of the nozzle exposure sheath. In some embodiments, the computer system 101 is an electronic device of a user or a computer system that is remotely located with respect to the electronic device. In some embodiments, the electronic device is a mobile electronic device. In some embodiments, the electronic device is located within the catheter device.

The computer system 101 includes a central processing unit (CPU, also “processor” and “computer processor” herein) 105. In some embodiments, the CPU 105 is a single core or multi core processor. In some embodiments, the computer system 101 includes a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage, electronic display adapters, or any combination thereof. In some embodiments, the memory 110, storage unit 115, interface 120 and peripheral devices 125 are in communication with the CPU 105 through a communication bus (solid lines), such as a motherboard. In some embodiments, the storage unit 115 is a data storage unit (or data repository) for storing data. In some embodiments, the computer system 101 is operatively coupled to a computer network (“network”) 130 with the aid of the communication interface 120. In some embodiments, the network 130 is the Internet, an internet, an extranet, or any combination thereof, or an intranet that is in communication with the Internet, an extranet that is in communication with the Internet, or any combination thereof. In some embodiments, the network 130 in some cases is a telecommunication network, a data network, or any combination thereof. In some embodiments, the network 130 includes one or more computer servers, which enable distributed computing, such as cloud computing. In some embodiments, the network 230, in some cases with the aid of the computer system 101, implements a peer-to-peer network, which enable devices coupled to the computer system 101 to behave as a client or a server.

In some embodiments, the CPU 105 executes a sequence of machine-readable instructions, which are embodied in a program or software. In some embodiments, the instructions may be stored in a memory location, such as the memory 110. In some embodiments, the instructions are directed to the CPU 105, which subsequently program or otherwise configure the CPU 105 to implement methods of the present disclosure. Examples of operations performed by the CPU 105 include fetch, decode, execute, and writeback.

In some embodiments, the CPU 105 is part of a circuit, such as an integrated circuit. In some embodiments, one or more other components of the system 101 is included in the circuit. In some cases, the circuit is an application specific integrated circuit (ASIC).

In some embodiments, the storage unit 115 stores files, such as drivers, libraries and saved programs. In some embodiments, the storage unit 105 stores user data, e.g., user preferences and user programs. In some embodiments, the computer system 101 in some cases includes one or more additional data storage units that are external to the computer system 101, such as located on a remote server that is in communication with the computer system 101 through an intranet or the Internet.

In some embodiments, the computer system 101 communicates with one or more remote computer systems through the network 130. For instance, the computer system 101 communicates with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. In some embodiments, the user accesses the computer system 101 via the network 130.

Methods as described herein are implemented by way of machine (e.g., computer processor) executable code stored on an electronic storage location of the computer system, such as, for example, on the memory 110 or electronic storage unit 115. In some embodiments, the machine executable or machine-readable code is provided in the form of software. In some embodiments, during use, the code is executed by the processor. In some cases, the code is retrieved from the storage unit 115 and stored on the memory 110 for ready access by the processor. In some situations, the electronic storage unit 115 is precluded, and machine-executable instructions are stored on memory 110.

In some embodiments, the code is pre-compiled and configured for use with a machine having a processor adapted to execute the code, or is compiled during runtime. In some embodiments, the code is supplied in a programming language that is selected to enable the code to execute in a pre-compiled or as-compiled fashion.

Aspects of the systems and methods provided herein, such as the computer system 101, are embodied in programming. In some embodiments, various aspects of the technology are thought of as “products” or “articles of manufacture” typically in the form of machine (or processor) executable code, associated data, or any combination thereof that is carried on or embodied in a type of machine-readable medium. In some embodiments, the machine-executable code is stored on an electronic storage unit, such as memory (e.g., read-only memory, random-access memory, flash memory) or a hard disk. In some embodiments, “storage” type media includes any or all of the tangible memory of the computers, processors or the like, or associated modules thereof, such as various semiconductor memories, tape drives, disk drives and the like, which provide non-transitory storage at any time for the software programming. In some embodiments, the entirety of the software or portions of the software, at times, is communicated through the Internet or various other telecommunication networks. Such communications, for example, enable loading of the software from one computer or processor into the other, for example, from a management server or host computer into the computer platform of an application server. Thus, another type of media that bears the software elements includes optical, electrical and electromagnetic waves, such as used across physical interfaces between local devices, through wired and optical landline networks and over various air-links. In some embodiments, the physical elements that carry such waves, such as wired or wireless links, optical links or the like, also are considered as media bearing the software. As used herein, unless restricted to non-transitory, tangible “storage” media, terms such as computer or machine “readable medium” refer to any medium that participates in providing instructions to a processor for execution.

Hence, in some embodiments, a machine-readable medium, such as computer-executable code, takes many forms, including but not limited to, a tangible storage medium, a carrier wave medium or physical transmission medium. Non-volatile storage media include, for example, optical or magnetic disks, such as any of the storage devices in any computer(s) or the like, such as are used to implement the databases, etc. shown in the drawings. In some embodiments, volatile storage media include dynamic memory, such as main memory of such a computer platform. In some embodiments, tangible transmission media include coaxial cables; copper wire and fiber optics, including the wires that comprise a bus within a computer system. In some embodiments, carrier-wave transmission media takes the form of electric or electromagnetic signals, or acoustic or light waves such as those generated during radio frequency (RF) and infrared (IR) data communications. In some embodiments, common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code, data, or any combination thereof. In some embodiments, many of these forms of computer readable media are involved in carrying one or more sequences of one or more instructions to a processor for execution.

The computer system 101 includes or is in communication with an electronic display 135 that comprises a user interface (UI) 140 (alternatively called a user interface (UI) module elsewhere herein) for providing, for example, a real time pressure reading, a real time temperature reading of the tissue, a real time temperature reading of the ablation medium, and a real time location of the ablation balloon, catheter, or any combination thereof once it is inserted into an individual. Examples of UI's include, without limitation, a graphical user interface (GUI) and web-based user interface.

Methods and systems of the present disclosure are implemented by way of one or more algorithms. In some embodiments, an algorithm is implemented by way of software upon execution by the central processing unit 105. In some embodiments, the algorithm, for example, calculates a real time projected subcutaneous needle location prior to insertion, acquires a plurality of voltage signals, and converts them into a pressure sensor array.

### EXAMPLES

### Example 1—Gallbladder Defunctionalization Using the Catheter Device of the Disclosure and a Thermal Ablation Medium

An 80 year old individual presents with severe pain and tenderness in the upper right quadrant of her abdomen that has lasted for several hours. The physician diagnoses the individual with cholelithiasis, but given her age, the physician determines the individual is at high risk of surgical complications. The physician therefore chooses to percutaneously defunctionalize the gallbladder of the individual using the catheter device disclosed herein, instead of surgically removing the gallbladder. In some embodiments, the gallbladder defunctionalization device disclosed herein is used treat the gallbladder of the individual affected with gallstones.

The gallbladder is accessed by a transhepatic or subhepatic interventional radiology (IR) procedure at the bedside. The guidewire of the catheter device is placed into the common bile duct of the patient. The catheter device deploys a plug into the cystic duct of the individual. The plug temporarily prevents the bile produced in the liver from entering into the gallbladder (e.g., the plug prevents bile from entering the gallbladder during the procedure).

Then, an ablation balloon catheter is used to deploy an ablation balloon to the lumen of the gallbladder of the individual. Next, the ablation balloon is inflated with a thermal conductive ablation medium within the gallbladder. Next, the thermal conductive ablation medium is heated to about 80° C. and the outer surface of the ablation balloon comes in contact with the superficial surface of the gallbladder for about 8 minutes, thus ablating mucosal layer of the gallbladder. After the ablation is completed, the ablation balloon is deflated, and the gallbladder defunctionalization device is withdrawn from the gallbladder and the individual.

### Example 2-Gallbladder Defunctionalization Using the Catheter Device of the Disclosure and a Cryogenic Ablation Medium

A 78 year old individual presents with severe pain and tenderness in the upper right quadrant of her abdomen that has lasted for several hours. The physician diagnoses the individual with cholelithiasis, but given his age, the physician determines the individual is at high risk of surgical complications. The physician therefore chooses to percutaneously defunctionalize the gallbladder of the individual using the catheter device disclosed herein, instead of surgically removing the gallbladder. In some embodiments, the gallbladder defunctionalization device disclosed herein is used treat the gallbladder of the individual affected with gallstones.

The gallbladder is accessed by a transhepatic or subhepatic interventional radiology (IR) procedure at the bedside. The guidewire of the catheter device is placed into the common bile duct of the patient. Standard Seldinger technique, bore needle+wire. The catheter device delivers a cystic duct ablation medium (e.g., nitrous oxide) into the cystic duct of the individual in order to chronically occlude the cystic duct. The cystic duct ablation medium delivery induces scarring that further permanently prevents the bile produced in the liver from entering into the gallbladder.

Furthermore, a catheter comprising a fenestrated nozzle comprising a plurality of fenestrations is introduced into the lumen of the gallbladder of the individual. Next, the fenestrated nozzle is used to circumferentially spray nitrous oxide, a cryogenic ablation medium, within the gallbladder for three cycles, each cycle lasting about 1 to 3 minutes at a temperature of about −80 degrees Celsius. As a result, the nitrous oxide ablates mucosal layer of the gallbladder. After the ablation is completed, the catheter is retracted and withdrawn from the gallbladder and the individual.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein are employed in practicing the disclosure. It is intended that the following claims define the scope of the disclosure and that methods and structures within the scope of these claims and their equivalents be covered thereby.

1.-20. (canceled)

21. A gallbladder defunctionalization device, comprising:
an access sheath defining a first lumen, the access sheath having a distal end configured to be disposed in a gallbladder lumen of a subject;
a catheter disposable within the first lumen and having a distal end extendable distal to the access sheath, the catheter including a first expandable body disposed on the distal end of the catheter, the first expandable body configured to transition into an expanded state within the gallbladder lumen, the catheter defining a second lumen configured to deliver a cryogenic ablation medium into the first expandable body in the expanded state,
the access sheath including a second expandable body disposed at the distal end of the access sheath, the second expandable body configured to expand to retain the access sheath within the gallbladder lumen after the distal end of the access sheath is disposed in the gallbladder lumen;
a pressure sensor configured to measure a pressure associated with the gallbladder lumen; and
a control unit operatively coupled to the pressure sensor, the control unit configured to, in response to the pressure measured by the pressure sensor reaching a threshold pressure, control at least one of a delivery of the cryogenic ablation medium to the gallbladder lumen or an evacuation of the cryogenic ablation medium out of the gallbladder lumen,
the first expandable body in the expanded state including a plurality of fenestrations that are configured to allow the cryogenic ablation medium delivered into the first expandable body to exit the first expandable body and contact tissue of the gallbladder lumen,
the first lumen of the access sheath configured to evacuate at least a portion of the cryogenic ablation medium from the gallbladder lumen after the cryogenic ablation medium is delivered,
the catheter movable relative to the access sheath to control a location of the delivery of the cryogenic ablation medium relative to a location of the evacuation of the cryogenic ablation medium.

22. The apparatus of claim 21, wherein the plurality of fenestrations span a circumference of the first expandable body and an axial length of the first expandable body.

23. The device of claim 21, wherein the second expandable body includes a deployable Nitinol structure.

24. The device of claim 21, wherein the second expandable body when expanded has a diameter that is larger than a diameter of the access sheath.

25. The device of claim 21, wherein the second expandable body encircles a circumference of the distal end of the access sheath.

26. The device of claim 21, wherein the access sheath is insertable into the gallbladder lumen via an opening in a wall of the gallbladder lumen, the second expandable body configured to create a seal between the access sheath and the wall of the gallbladder lumen near the opening.

27. The device of claim 21, wherein the first expandable body in the expanded state is configured to contact at least a portion of a wall of the gallbladder lumen.

28. The device of claim 21, wherein the plurality of fenestrations is a first plurality of fenestrations, the catheter further including a second plurality of fenestrations disposed at the distal end of the catheter and configured to deliver the cryogenic ablation medium into the first expandable body.

29. The device of claim 21, wherein the first expandable body is an inflatable balloon.

30. A gallbladder defunctionalization device, comprising:
a catheter having a distal end configured to be disposed within a gallbladder lumen, the catheter defining a lumen that terminates at a nozzle;
a first expandable body disposed around the distal end of the catheter, the first expandable body configured to transition into an expanded state within the gallbladder lumen, the first expandable body in the expanded state having a diameter larger than a diameter of the catheter and including a first plurality of fenestrations in fluid communication with the lumen;
a pressure sensor configured to measure a pressure associated with the gallbladder lumen;
a control unit operatively coupled to the pressure sensor, the control unit configured to, in response to the pressure measured by the pressure sensor reaching a threshold pressure, control at least one of a delivery of a cryogenic ablation medium to the gallbladder lumen or an evacuation of the cryogenic ablation medium out of the gallbladder lumen;
an access sheath; and
a second expandable body disposed at the distal end of the access sheath, the second expandable body configured to expand to retain the access sheath within the gallbladder lumen after the distal end of the access sheath is positioned in the gallbladder lumen,
the nozzle including a second plurality of fenestrations, the lumen configured to deliver the cryogenic ablation medium through the second plurality of fenestrations and into the first expandable body when the first expandable body is in the expanded state such that the cryogenic ablation medium can exit the first expandable body via the first plurality of fenestrations and contact and ablate tissue of the gallbladder lumen,
the first expandable body in the expanded state configured to space the second plurality of fenestrations from a wall of the gallbladder lumen such that the second plurality of fenestrations are centrally located within the gallbladder lumen for distributing the cryogenic ablation medium.

31. The device of claim 30, wherein the first plurality of fenestrations span a circumference of the first expandable body and an axial length of the first expandable body.

32. The device of claim 30, wherein the first expandable body in the expanded state is configured to contact at least a portion of the wall of the gallbladder lumen.

33. The device of claim 30, wherein the lumen is a delivery lumen, the device further comprising:
an evacuation lumen configured to evacuate at least a portion of the cryogenic ablation medium from the gallbladder lumen after the cryogenic ablation medium is delivered.

34. The device of claim 30, wherein the second plurality of fenestrations span a full circumference of the nozzle of the catheter.
